SEQUENCE LISTING (57 sequences)

SEQ ID NO: 1 - DNA, 34679 bp, synthetic construct
  misc_feature 1..34679: note = P08-G05-PacBio_cluster016
  source 1..34679: mol_type = other DNA
gatgtacgaggtggcgctgttgagcatggcggcgatgccgatgccgatcagcacgagccgggtgccggccaccccgtccttgaacgacagcaggtagatgaggacggccacgccgagcgcggccacaatggcgaagatcgagacgcccgtctcaccgaggccgagcacgatgatgccgaaggccgcggcggcgctcgccgccgagctgatgccgatgatgtcggggctggcgagcgggttgcgcaacatggtctgaaaggtgacgccgcccatgccgaagcagagcccggtcgccagggccaggaccgtgcggggcagccggagttcaccgacggtgaacgaggcgccgggcacggtgtcgccgaggatcacccgcagcacgtcgccgggcgggtagaacgtctgcccgaccatcagcgagacgaccgccatgacggccaccgccacgccgagggtggtcagcacggcgcgtcggcgtcggctgcgccggacccggccccgggtgacggcctcgaaggtcgtggtgctcacagttcgcgtaccttctgccggcggacgatgtagatgaagaagggggcgccgatcagggcggtgatgatgccggcctcgatctcgtcggggcggttcaccacccggccgacgacgtcggctgcggtcagcagcgcggcgccgaccagggtcgcgaacggcagcagccaacggtggtcgagtccgacgaggagtcggcacaggtgcgggacgatcaggccgacgaaggcgatcggtccggcgaccgcggtggctgcaccgcagagcaccaccgcgccggcggcggcgatcccgcgtaccagggcgacgcgttcgccgaggccggcggcgagttcgtcgccgagtgcgagggagttgagcgcccgggtgcacagcaggcagatggccagcccggcgatcaggaacggcaggacctggccgatgctgtcccagctggcgccgccgacgccgccgacctgccaggactggaagttctcggcgatgtcgccgcgcggcagcacgaccgcgaccaccagcgacgcgagggcggcggaggtggccgccccggccagggcgatcttgagcggggtggccccgccgcgaccgagggagccgaccgcgtagacgaacagtgcggacaccgccgctccggcgatggcgacccagatgtacatggtggcggtgtacaggccgacggtggtcatggcgacggcgatggccagcatcgcgcccatgttcaccccgaggatgcccgggtcggcgagcgggttgcgggtgacgccctgcatcacggcgccggacagggccagcgccgcgccgaccagggccgcgagcaccgtccggggcaggcgtttgaccacggctgcctggtcgagggtgtcgtgggtgccgccgagagcggcggtgatgtcggcccagccgacgatccgggagccgaaggcgaccgacgcgatcagggtcaggaccagcacggcgaggacgaccaggagccaggtcagccgcgtccgggccgggcgtcgcaccagggcgacgcccggccctgatgtcgtttcgcaggtcatcagagcttgtcggcggctttgccgagcaggtcggcgtagtccttgaggacgaacgagacgcccaggggcgtcgggttggcggcggtggccaccggggtgctgccgggcagcgagacgaaggagccgcgcttgatcgccgggatcttggacagcagcgggtccttctgcagggtggccagggtggtgccgttggcgtcaccgtaggtcacgatgatgtccacgtcggcgaggttctgcacctgctcggcgctctgggtgaggctgaactccttggtcgccgcggaggccttcgcgatgctctccgggtgcgtcatgccgaggtcggtgaagaactgggcccgggtgtcgtgcgtggtgtagaagctgatcttgctgaggtccgtcgggtcgaggtgggtcaggaacatcgccgacctgcccgcgagcttggggtagcgcgcggcctcgtccttcatctgcttctcgatcttggcgatcagcgcgtcgccctcggtggccaggccgagggcccgactctccagcttgatgctgtcgcgccacgacgtggcccagggcgcctgcgggtacgcgacgaccggcgcgatcttggtcagggtgtcgtagtcctgcttcgtcagaccggagtaggcggccaggatgacgtccggcctggtgttcgccaccgcctcgaagtcgatgccgtcggtctcgtcgaacagcaccggtgtcgacgcgcccagttccttgagtctcgcggcgtcccagggcagcaggccgtcgccgtcctcgtcgccgaagttggccttggcgtagccgaccgggacgatccccagggccagggggacctcgtggttggcccagttgacggtggcgacccgctcgggcttcttctcgatggtggtgacgcccaacgcgtgggtgacggtcagcgggaacgtcgccgtcgcggacccggacgcggcgggcttcccgtcggggtcggacgacccgccgcaggcggcgagtgcgagcgtggtggtgaccgcgacgatgccggtgagcaaccgggagaggcgcattcggagtactccaactcacatagtaagggcaggcttacctaatacttgatcggtaaagtaccccccgttcctgagccgttgaccagggggtacgaggcagtgagcaagatcagtcccggcaccgccatcctgcgcagggcggtcgcccgcaacgggcgacgcctctggggtggaacggcgctggccggcgtctatcagctctgcgcggccctggtgccgattctcatcggtgtcatcgtcgaccgggccgtcgccaccggcgacctcaccgcgctgctcacctggatcacgaccctggcgatggtctacctcgtcctggcggtgacgtaccgcctcggcgcgcggcaactgcaacgcgccatcgccgaggagggccaccggctccgggtggaactggcggcgaggatcctggacccgcgcgtgctccgcaccgaacggcacaccggcgacctgctctcggtcaccaccaccgatgccgagtacacctcctacctgctcgaccacattccacggatcaccagcgcgctggtggcggtcaccgtcagcgcggtcgcgctgctgctgatctcgccgccgctcggactcaccgtgctggtcgccatcccgatcgtgctggcgatcctcaacctcaccgcaccgctgatcgcccgacgcgtcaaggaacagcaggaccaggccggccgggccaccgcgctcgccacggacctggtgaccgggctgcgcccgttgcgcggcatcggcgcccaggacgccggcgccgcgcgctaccgccgggtcagccgccgatccctggcggccgccctgcgcgcctcccggacccagaacgcgtacctgagcgcgtccacgatgtcgagcaccctgctggccgggggcatcgccgtcatggcgggctggttcgcgctcaccggtcgcatcacggtcggccagttcatcacggtgatcggctccgcgcagtttctcgtcgaaccgttcggcgtgctcgcggtcgtacccagttggaccgccgccgcccgcgcgtccgcggaccgggtcgccgccgtcctccaggccggggtggtgctgccgccgggcgtcgccgcgccccccgacaccgggtacgagctgcgcctgaccgacgtgacgtacggcccgctggccggcctccacctgcacgtccgcgtcggagagttcgtcggtgtggtggcgcgccggccggcggacgccgaggcactggcgcggctactggcggtgcccgacgcctaccagggggacatcctgctcggcggagaacggctggagaccgtggagcgggaccgggcgcgccgcgtgctgcacagcgaaccgcaccgtaccgatctgttcaccggcaccctcgcctcgaacgtcgccatccacgacgacgacggcggacggctggccgcggccctgcgcgccgctgccgccgacgaggtggcacacctgcatccggcgggcctgcacatgccggtcaccgagcgcggcgcgaacctctccggcggccagcgccagcgtgtcgcgctcgcccgcgcgttgctcgccaggccgccgctgctggtcctgcacgacccgaccaccgccgtcgacgcggtcaccgaacaggccatcgccgacggcatccgggcgctgcggcacggaccggacgccagctacggcaccgtcctcatcaccagcagcccgacgctgctggccgccaccgaccgggtggtgttcctcggggacggcaccgtcaccgccgagggtgcccacgccgaactcgtcacccgcgacgacagctaccgccgggcggtgcggcgatgacgcacccgaccctgcccgtggccagcccgcgcgagagcagggcgtggctcggtgcgcaactgcgcgcccgcgcggtggccaccggcgccaccctggtcgtcgggctgctcgccgccgccaccgcgatcgttcccgcctacgccctcggcctgctggtcgaccgggtccgggccggtgccggcaccgacgcgctcgtgccggtcgccgtggtcatcggggccgccgcagtcgtcggtggtctggtcgccggcgtcagcgcccacctgatccgggggttgggtgcacggatcctcgccgacctgcgtgaacgcgccctggacgccgcgttgcgcctgcccgtggcggtgctcgaccgggtgggccggggcgacctggtctcgcgggtcggcgcggacatcagcgccatcgaccgggccgtctccgaggtcctccccaccatgatctcggcactgctgctggcggcgatcagcctcggcacgatcatcggcatcgactggcggctggggctggccggcgcggtcgccgtcccgctgtacgtgctggggttgcggtggtacctcccccgggccgcaccggcctacgcggccgagcgggtcgccatcgcacagcggtcccagcagttcgtcgagagcgtgcagggcctgcggacggtgcacgcctaccggtgggaggatcggcacctcgccggcatcgcggctgcctccgcccgtgcccgggacatctcgacgagcgtgttcgccctctacacccgtttcgtggggcgcatcaacggggcggagctggccgggctcggcgcggtgctggtggtgggattcctgctcgtacgccgggacgctgtcaccctcggccaggtctcggcggccgcgctgctgttgcaccgcctgttcggtccggtccttcagctgatggtcaccttcgacaaggcccaggacgcgggggccagcctcgcccgcctcgtcggggtgctccgcaccgaccccgaacgccgcgcgggcggcaccacgcccccggcgcacgacctcgtgctgaccgacgtcggcttcagctacgacgggaccacaccggtgctgcgcgacatctccctgcgggtcgggccgggccaactggtcgcgctggtcggcgccaccggcgccgggaagaccaccgtcgcatcgatcgccgccgggatcctgcgccccgagcacggggcggccaccctgggcggcgttcccgtcaccgagctgcccgcccggacgatcgccctggtcagtcaggagacccacgtgttcgccggtccgctcggcgaggacctgctgctggcccgcccgcacgcctcgccggaggagctcaccgcggccctggcccgggtcggcgccctgtcctgggcgctcgccctccccgacggcctggacaccgtggtcggcgagggcggccaccctctgaccgcggcgcaggcgcagcagttggcgctcgcccgggtggtgctgctcgaccccgcggtcgtggtgctggacgaggcgaccgccgaagccggcagcgcgggggcacgcgacctcgaacaggcggccgccgcggcgctggagggacgcaccgccctggtcgtcgcccaccgcctcacccaggccgtgaccgcggatcacatcatcgtcatggaacagggcaggatcgtcgagagcggcccgcacgccgacctggtcggcgcgggtgggaactacgcccggctgtgggcggcgtgggccgaccgcgccaccgccgacccggggccgacccgcaccggggccgcagcggggcgcgcggtggacagcggcggcccgtcgacaccgcaaccgcggcacgggcggtggaccggaccgccgctgaccgctcagcgggagtagaactccaccacgagttgctcgtcgcagatcaccggcacctcgtgccgggcgggctcgcgcagcagcgtggcactcaacccggtcagctccaccgacaggtacggccggggtcggtcgtcggcgtgcgcgccggcggcggcgagctggaacggtggcagggcacggctgcgctcgcgcaccgcgaccacctggccgggccgcagccggtacgagggccggtcgaccttgcggccgtccacggtgaagtggccgtgcccgacgagctggcgcgcctggtagatgctgcgggccagcccggcccggagcaccagcgcgtcgagtcgccgttccaggagcgcgaccagcgactcgccggtcttcccggcgccccgggcggcctcgtcgaacgtccggcgcagctgggcctcgctgacgttgtactggtggcgcagccgctgcttctccagcagccgcacctggtagtcggaggtcttgcgacggttgcggccgtgcacgcccggcgggtagggacgccgctcgaagtaccgcacgcacttgcgggtcaatgggatgccgagcgcgcgggagagccgggccttgggtcgggggtggttcacgtcgacgtctccgatcgaatggtctagcctgcaacttaggtaaggctaacctacattgaggagttcgggatcatgcgacccagcccggcggagatcgtgcgtaccctcgtcgcgggccggctgcccgccctcgcccacctggccggcagcaccggcccgcaccaggtccggcacgccaccgacccggacggccgggtgctgctgctggtgccggcgctcagcgacctcgccgcggcgctcgcgccgccggccggggacaccgacgtcgccaccgccctcgacgtgctcgacctgccgcccaccgccggcgccccctcgctcggccgggcctgggtctccggctgggccgaacgcctcgacggcgacgaggcacgtcaggccgcgatggacttcgccaccacccagccgaccggcgacctgctagacgtgggcagccggttccagctgcaccggttcgaggtggccgaggcccgctgggaacacaccggcgggatacgcccgatcgacccgcaggcgtacgcgcaggccgagcccgacccgctgcacgcggccgaggcgggattgctggccgacctcgccgaccaccacgccgagcaggtcgcgggccacctgcgccggcagttgaagctctccggtgccgagtcgccacgggtggtccggatcgaccggtacggcctgctggtcgcgtacggccggcccggtgcgacccgacgggcgagggtgagcttcgcgggacccgtcgccgacgtcgccgagttgtcccggctgctgcacccgatgctctgcccccggcaggcggccgcctgaccgcgccacgaccggcccggcccgtcacgcccggcggccggcacggccctcgggtcgtcgttgttccgcaccggcccccgccacctggggttcgatcccgcgccgcacgggtttcccggcgggcggcccgcacccgtccgggctgagccgcgactcagatcgggagctgcgggccgagcacggcgcccgggtcgtagcggcgcttgacctcccgccaccgctcgacggtgggcccaccgaactgggcccggtagaagtcccgctccacccggtgatagccgtgcaggtactgccggccgccgagctcgtgacagagcgcgccggcagcgtcgaaatgctcccggtactcgtcgacccggtccggcggcacgatgcagtagaagccgaacgtgcgtacccggtcggcgtcgggcagcggactcaacggcggccggggccggtccccggcgcgcaggaatgcgccgttgaccaccgggaccacggcgggatcggcgaaccggtcgcggaccccggcgaagaaggcgtcggccttctccatcggcaagaagaagtcgctccacagccggcgcgcctcggtctcgtccgtcagcagacccgcggcaacctggccacgggcgatgccgacggagaagcccgcctccgcctcgaacagccgctggtgatagtcggcgaccagggtctcgtccgactcggccgccgcgtcgcgcgccgaccggccgacctcgaccttccaccggcgcgcggcggtggagtacaccgcgcggcacagacgcagccgctcgtcagcccacacctgctccgccaccgtggcgaggtcgtcgccgtctagccggcggaccaggacggccggttccgccgggaccacccgcagctcggcccggacgatcagacccacctgaccgagaccgcagagcgcgtacgagaacagctcccggtgcgggccgtcggcggtcgcgtgcacgagtgcgccgtcacccgtgacgacctccagggccaccacctgatcgacgaccgcgccgacgaagggtgacgtcgggccgaagccgccgaccgacagcgtcccgccgaccgaggcgtgctcgttgctggtcacgaccggcagcgcgaggccgtgctcgtgcagccagcggtgcagtgttccccagcgcaccccggcctcgacgcgcaccgtcgccgccccgaccagttcgatccggtccaacgcggcggtgtccaactgcaggccgtcggtgaggctctgcccgttgctgctgcacccgttgccccgtgcgacgaccggcaggccgccgacgagcgcgtagcgcaccgtctcctgcacgtcggcgaccctggcggcgaacacgaccgcctgtggctcctggcagcgcagccggccgaagtcccgctgccatccctcgggtgccggatcgaccagcttcccggccaggagccgggcgagcggttgatgtggcgtcatcagacggtcgccgtcggccggctgatcaggcccttgagcttcgccgcgaagtggtcgaagaacggcaccaggtcgcagaccgggtcgccctgccgcaccgccgcgacggcccggtcgcgccactcgtcgaccgcggcggcgacgtcggccaggtcggccgcgcccgaggcgaccaggtgggcgaggttgccgtcgcgctcgtcgatctcgtcgaagtcgaccgcgtcgtcgccgatcatgtacagcacgcccagcgcctcaccgaactcccgggccgccgtcaccatctcgggaaccccaccggccaggcagttgaccgcggcgtagttggcgagcatcaccccggccttgatccgggccgccgcgatccaggactcgaccgtggtcggtcggggcgcgctgacctccacgatctcctgccgccagatgtcgcggtagtcgtcctcgagcgtcctgagcacctcgaccggccgctgctggctggccagcagtgtggtgccgaccgagagcagttggaccccgaagatctggtcgcgcggcgagagcggctggtcgtcgtcgatgaggtcgtcgatcagcttcacgccgaccgcgtggatcgagaccccggcgccgaggtcggcgcgcacccgcgggtcgcgcacccagagcgtcgtgtagtacggcgtacgcgggtagccgggaaggtcacgcacgaagtccagtccctgccgggacatgcccagactggtcagctcggcgagacaccgttggaccacccgcgcctggctgtcgtccatctcggccttgaactcaggaaacgtcaacacggtcgtcctccaggagaaaggtgatcaggcggtcggcgaagtcggccatgaagccggcgatcgggaactccggcgggaactccgccagggcgcgcagcgcctccgcgcgccaccggcgggtcgccgcgacggccgcgtcccggctcacctgcccgctccggatcaggtggcacaggtttccgccggcctcgccgaggtcgcggtggtcacggatgtcgtcgccgatcatgaacaggacaccgaacgcctcgcagaacgcgcgcgcggcgggcagccgctccacgacatcggcggcgaggcaggcggcggaggcgtaacacatcatcatccggcccgacttgatgcgggcgtagtggatccagctgtccaggtcgtgggccggacgttccgcctcgttgatctgctcccgccagatcacctcgtagtcctcgtcgaagacgcgcagcacctcggccggccggtggtgggccccgaaggtcgccgtgctgctctgggtgaggaacatgccgaggatctggtcccagggccggatcggttggtcgccgtccatcaggtcgtcgaccagtttgatccccatcgcgtgggtgcagatcgcgatccccaactcctggcgctgcaccgggtccggcacccagagcgaggcgaagtagggaatccgcggatagcccttcatctggcggacgtactccaccccgtccggggtcatgccgatcccggcgagccggtcggtgacccgctccagcaggacgcgctgcgcgaccgcccactcgtcgtccagaccgctcactccggagagcacgtttcctcctcgtcgatccgcagcggcaccccgtcgagcactgcggcgaccgcggcgacggtctgccgccgggcccggtccgggtcgagcagcgtacgggcgagggccatcgcccggcggcggcgacggggcagctcatccgggtcgcccacccagcggacggcgacctcgaccaactcctccatccggtcgaagccccggccgacgccgtgttcggcgaggaacgggccgacccactcctccatcggcaggccggagcgcaccagcagttgcgggacgccgaagtagagcccctccaacatggagttcggcccggtcttggagatgaacagcggcgcgtcggcggcgtggagccaggccaggtaccgcaggtagtccccctgcgccagccggtcgaaggtctgccaggccgaccggcccagcgcggtcagcagttcggccgcctcggcggccagcgccgggttgttgtacgcgatgaagaccagcgccgcctcgggatgcgcgcgggcgatccgctccagggcggcgagatactcccggccgccccggttggacaggatcaggatccgggtgccggccgccgtcaacgccggatcggcggcaccgacggcgtcggcgtccccgatgcctgcggcggcgccctcgaagacctgggcggcctgggcctgggcgagcaaccggccgaccggcacgacccgctcgggccggtagccgtgctcgcggacgagatagtccttggcccggtcgaactgcaccaggtggaccggcaggtggggcgcccggtgcaccggcagccgcagcagcccctcgttggtcacgtagttgaccaccggggtcggcaggtccgcgatccgcagcgcggcatggctcagccgggagatcacccccttggtgccgatcaccaggtccggtttcgtctgggcgaagtggtcggcgacgggtgccagccgatcgacggtcgccgcgggcagcgggtcgtcgtccaggcccgccggtagccggtcggtgaagtccgtcatgaggcgcaggtatccggcccacacctcgtcggcgggcagccgggccgccaccagctcacgaatggcggcaccggcctcggcgaactgcgggaagagcgtctccgcctcgacgacggtgacctccgcatcgtccagaccggcggccagggtcgcggcgagcgtccggccggcgaggagattccccagctcaccccaggtgcgcatcgagatgacctgcacgctgcgggtcatgcccactccctccgtcgcgctcgtggtgtcatgccagcagtcccgggttgaacaggccgtgcgggtcgagttcccggcgcaaccggcgtacggcggcgaggtcggcgccgtacatcgactgcaggtccgcctcgtccaggtcgtaccagccggccaggtagggccggccgcccagcgcgaggcactcggccagcacctgacggtgcagcccctgcacccgtgcgacgccggccccgtccccgcgcgggacgctgaagtacagcccgaagccgacggccaggccccgtagtccggccgccggggtgaacggcacccccggacgttgctccgggcggtgtacgccgagcatgagcagccggcccgcaccctcgcggtagccggggctcggcaggatctccgcgtcgaggaaccgggcgaactcggctgccgccggcagcgacaggcagtagtcggcccagatgaagtgcaccgacgggtcggccggattgcgccgggactcccgcatggaccgcaacggccagtcggcctcgacgcgggtctcggtctgtcggcgcagttgcggcggcagtgggccgtcgccccgcatcccgtacgtggcgatgaagccgccctcgtagtgctgggcggagaagaggtcgaccggcgaccgctgctcctcggccagccacccgatgctgccgaccagggcggtcagtgaggggtgccgccgcgtcgcgatggtggtgtgccgctggtagtcgaccgtacgcaggaccacccggtccaacacgcccaactggccgagcgagccgatgccgtaccggaacagctcgggcgactcgacgggcgagcaccaccgggcctgcccgtcgggcaggatcagccggaaccgctcgaccaggtcgacctggccgccgtacctgatcgtgccctcgccgtacccgccgaccgacagcgtgccaccgatcgagaccgtcagatggttggtcagcaccgggatgctgcgcccctgggcgttcagggcctgctcggcggcgtaccagggtgcccgggcggacacctcggcggtgccttcgtcgcgcagccgaaccccggggtcccccggctcgccgacgatcagcacgccaccgtcggacaggccctgcaggccgaaggagtgtccgctgccgcgcaccgcgacgggcgtcccggcggcgcgggtggtccgcaggacgtgcgcgacctcctccgccgaggtcaccggggccaccacggccggcagcagctcgtgcagacggcccacgttcgtggcgtacgcctgccggccgggcagatcggtgcgcacgggccggcgcagcccgcgctgcaactcggcggcgagtgtcgggggggcagcggggtcaccggcccggtcgcccggccggccggacaacctgcgcagggtctcccgccagtcggcggtctccaggccgttgccgacgacgacccagtcgacgccgcagtcgtgatactcccggacctgacggtacgaggtcacgccaccgccgacgaacagcaactgctggggtgccaggccacgccggtaggcccgcagcaccgcctcggggacccggccgtgccggtagagcaggtaggcgaggtcgaagtcggcggcagcgaccgccgccaccagccggggcagttccgcgccgtcctgcaccgtcggcggcgcaccgaccgcctcgtgggtcttgccgtcgctaccgaaggcgaacgacgccgatcgcagcagctcgggcccgtcctgcccgagcgccgtcgcggcgcggacggtcgcggtcaggccaccggagacgaagaaggaccgcaccgagttcggtaccgcgtgcaccagcaccgcggcggcgcccgggcagagcggagcgccccggccgggtatcgacgggaagtgcaggacgaggggcaggtcggtccgctcgcgcagttcggcgacgtaggggaccacctgtcggtcgaacgcggcgttgtcggtgctgccgacgagcagcgcgggatggccgaggtcggccaccgcccgcgacttctcgaccgcctcggccaacggcaccttgtacgggtccacgacgtgcagcagcccgggggcctgcgcccgcagcgctgtgagcaccggcgcggtcgccggtaccgtgctcagattcccgggaagtcgcacttcgcgcataggtcggtccgttcctttcgcagcaggctcgcgcggcgggcggtgtacttctcgttgttccagatctcctcgatggtgttgtcgttgacgttgcccatgacctcctgcacgctccagtcctggcagcacaggaccacccgaccgtcctgggtgatgtagaactcgttgaacggccgtacgcagaactggttgagcagcacggtgggacggcccagcacgttgccggcgcggttggtgagggtggcgtccggatcgcggtcgaccagcaccagctcgggccgggccggataccggcggaccttttccaggaccgacaggtcggtggtgtagtcgttgacgacgacggtggcgtaccgggtcagctcgtcggccagttcctcggtgagcttggagccattggtctggatggagatcgacgccttggtgcgctccttggccatccgcaccatcgcgggcagccgccggtccagcagcggctcgttgaacaggtacgggtgaatgacgccggtgaagtcggcgagctggtcgagcagcgactcgtagagctccatcgacatctggccggacggcatcttgagggtgccgatggggcagaacgagcagcggtagttgcagccgctcatcgtctcgatcgcgatcatgctgaactgcggtggcgtacggctggacttctgcttgagccaggcggcgtcgctgagcgcctttgttccggtgccgtggaagtaggtgcgggacactccgctgagcacgttgaggacacctttgccctgcgcgcgttgcttgatgtacttcgcgatgtctgtcatgcgcgttcccctcgggagatcttgggagacaacgggaccgacgaggccgttggtttggcgtgccgggtgtatgccgaccacgggatctcacgtcggggccggccggcgacgccgcgtactgcgagccccagtgccacccgtgagaaccagccgaaatggatcgatttgagcagccacttcttggccgcgaagtggaggccctggccgcgggctgcgggcgccagcggccacagcccgtagaagtacatgcgccagtaccggccggcgacgtcggcggggaagtcggccatcagcaccgccgcggtcagcagcgagcggttgacgcggtgtctgatctcgccggccggtccgccggtcaggtggtaactcacggcgtccgcgacgatgtgcgtgcggtgcagcgggttgtcgtgccgggggtcgagcagggcgaagcagaagtagtcgtcctcgcaggtgtagcggtccgggatgccacgggcggcgagttcggcgtcgatcaccatgcagcagccgtgcgggtagccgaacttcggcgtcttggtgatctgcttgccccggtgcagcaaccgggccgccgcgtacgggttgggctggggcaccttgctcgccccggccgagccccgcgcgccccggtcgaggaagtcctccagcagccgtccgatgcagccggcgctgaacgtcacgtcgtcgtcgacccacgccacgcccgccaggccgaggccggtggccacctcgaacgcggcgttgagcatgcgactcttcccggcctgcggcatcgcgatcccgacgaaggtgagcccgccccgggaggcggcgatctcgctcagctgccccagccgcgcgatcgcctcgtcgtgctggtcgtcgcgccactgcatgccgacgaccagcaccagcgggcgggcggccaccgtcggtgggaggtcggtgagttgggtgacgagttcctcgacggtctcgcggtggctcgacagcggcgcccggcccacgtaggtcggcaggaccaacgccagcccgccgccgaagccgtcgccgagttccgcggcgaccgccgggaccgccgtcgcggcccggacgccggccgggccgggacgaaccacgatgcactccgtgggtggtgggtcgccgtcggcgggttggtagccgcgccggatcgcccaccggacgggccgggtcagaagctcgagcatcggtacctccgcatgggtcagtcggtggctacggcggaccagagccggcgtatctcgtcggagctccccagcaggtcgtccagggccccgaccgcctccagccggccgtcgcgcaggaccacgacgagggcggcccggcgcagcacctcgggccggtgggacaccgccagcaccgtgctcccgccgtcgcgcaggatcccgtcccacagggcttcgctcgttgggggatcgagtgcgctggagacgtcgtcgcacagcaccagcggggtgcggcggacgagcatccgtgccacggccacccgttgcgcctggccaccggacaggcgtagcccacgaggcccgatcaccgtgtcgagcccgtcggccatcgccgtcaggtcggcgtcgagggccgaccgggacaccgcctcgtccagccggtcgtccggcagcccgagcaggatgttctcccggatcgtgccggtgaacagtcgaggcacctgcggcgtgtacgcgcaccggggcggcacgagcaccgcccggcggtccgcgggcaacgggacgtcgtcccagcgcacctcgccggtggcacccggcagttggccgagcagggcgcgcaacagtgtggtcttgcccgagccgacctggcccaccacgacggtcagccggccggccggcatcgtgaaaccgatgccggacaccccggtggcggcgccctcgtgccgggcggtgaggtcggtgaccgtgatcgtcggcgggccgccggcgggcggcgggggcgagcggcgcggcgaaaggttgatcatgacaccgggtcgggcgtggtcgaggtccatcccgtgccgggggtgcaggcggccacccgtgatcgcggtcatccggcgcagcgcgacgagcacccgctgcaactggacggtgagcacgccgagcgaggcgagcgtgtcgacgagcaactgcaggtagaaggtgaacagcaccatgtcgccgatggacagccggttgctgctgatggccggtgcggcgatcagcagcacgatgccggtgccgagcggggcggcgttcagcagggtgctgcgctgcaccgcgaccgcgagttcctcggagatggcgaccctggcccgaacggcgttgagcgcgtcgagacgccggccgaccgcgtcctcggcgcgcgcggcctgcacgcccagcgccacccccatcacctcgcgcagcgcctcgctgaccagtgcggtctgggcgcggctctgctcgcggtaggccatcatccgccggtgcaccaggcgcatcaccgccacgatcacgacgaacagcccgaggatgctcagggtgatgagcacgtcgatgcgcagcatgatggtgatcgcggcgaccgcgaacagccaacgggcgatgttgatgggcgaccacaccacgaagaagccggcgtccgcggcgtcggcgccgacggtcgcgaccgcctcgcccgcgctggtgtgtccggcctccggcggggggtcgatcagcgactcgaagagcgacagccgcaacgcgccgacggtgtggtattgcatccgcggttcgagccacggcaccaccgtgctgaactgcagggagatgcgcgcggcctcgatgccggcgacgagtgcgacgagagtccacaggttcaggccggcggccgactcgccgctgagggagtcgaaggccatctgcatcaacagccccagggccagcatgcccagccgcagcaccgcccacacggcggcgagcacggcgtaacgccggccacggccggccaacagcaggcgtgcccgggcgcctctggtcgtcgtggtcacgtcaggaccgcccggcggggaacagccgggcggctgtctcgtcactgccgcccgtcagctcggaccggggcgccaggcggacgatccggccgtcctcgacgaccatcacgtcgtcgacgatggagagcgtctcgggccggtgggcgatgatcagcgcggtacgaccctcgatgaacctctccatcgttcggcgcagccgccgctccgcgtgcgggtcgagccgggccgacggctcgtcgagcagcacgatgtcggggtcggtcagcagtgcccgggccagtgcgagcagctgctcctcaccggcggacaaccccgctccgcctggcccgatccgcgtgtcgaggccgtcgggcagggcggcgtaccaggtgtccaggccgacctcctcgaccgcccggcgcagttgctcgtcgggccaccggtcgtcgaagaaggtgaggttgtcccgcagggtggccgcgatgacgtgcacgtcctgggtgacgagcgtgatgcgggagcgcagcgcggcgacgtcccactgccggacgtccacgccgccgatcgagaccgagccggcctgcgggtcgtactggcggaacagcatccgcaggatggtcgacttgccgccgccgctgcgtccgaccagcgcgacccgccggcccgccgggatcgtgaacgacacgtccgacagcaccgggcgttcgtcgtaggcgaaggtgacgccggtgaagctcaccgcgccgcggcccggcgggagggtgccaccgccgtcggggacgatgtgcgcctcgccgaacagctcctcgatgcggcggacgctgcccaggacgttctccatgtcctggaagcgtgaggtgatgctgaccaggggcatccgcagcaggatcgcgtacgacaggccggcgaagacggtgccgacgctggcgccccggtgctggctgagccacacaccggtggccagtgccgcacagaggttgacggcggtgagcgcctggaccgtcgccggccaggtgaccgtggcccgggcggcgcggcgggccagccggtagtggtgcaggatccgttgccaggcgccctccagggcgtgcgccgtcccgccgttggcgttgaggtcctcccgcccggtcaggttctcctccagatagccgagcaggctcgcctccgcccgctggcggtcggcgatgagcggcaccgcgcggccgagcaggcgtcgcagcaggagcagcatcaccaccgcgagcggggtgaagagcacgccgtagcgccagtcgagcaggaacagcgccacgacgatgccgatgatcagcagcagttggacggcgatgtcgagcaccaactcggacaggagcttggccaaccgggtgacgtcgccgtccatccggtcgaccagttccccgggcgggtgccggaggaagaacttcggcccctgccgcaggacgtgcgcgagcaggtcgcggcgcaccccgttggtcgtacgccaggcgacccggccggagacccagccggtgccgaccgagagcacctgccgcacgaccgcgagcaccacgaagaccatggcgtacccggccaactcgccctgcgagcggccgcccaccgccgcgtcgatgaaccggcgtacgacctggggcgccaccaactgggtgccgacgtcgagcggtatcagcgcggccagcagcaggaaccagcgccggtgactgcggacgtagcccacatagcgcgcccagttcgtcgcggctgtccgcggctcgggagacgccatctggtcagccgtgcccggcgactgcgacccgggctgcgggtggcagctcggcggcgtcgccggtgggccaccggcccggtacggggccgatcccggtcagctcgtcgacctgcaaccggcaccacggcgagagggtcccgtcggtccgggtccggttccggaacgagtcccagtcgacccactcgacccggtccacctcgtccgggttcggcactggttcgtccgctgcccgggccacgtagacggggcacatctcgttctcgaccacaccgtcgagttcggcccggtagcggaagcggggcagcaccagacggaggtcgctcagcgtgaggcccagctcggcggacgcgcgtcggtggacggcatcggtcatcggctcacctgggccgggatggccgcagaagctgttggtccatatacctggccacgtctttttccgcaacgaacggcgcgacattaggaactgcccgctattgctgaagacgtaacaggagaatgcgagatgcagcggcgtgtcgccgtcgtgaacgatgcgtttgtcctgctcaccgacggcttggccatcgtggtcgagtagcacgaccagctccataccgctcccccaccatccggatcggtcaatttcgctcgcacaacaatccactgcgaatcgactccagtcaacccccctagaggaacaaaacgcccacacgggaaattgcggatgcgcgaagtagggggcgccggatctccacggatcggttacagtggacgttcaaccagccacggggggtcacgtaaatgcgggcccatccgaccctccgttattactgacagtgatcattttccgtcgacgtccggccgcgttctgccgcattcagcctgcgcgatcgttcacccggcggcaccggacaccggtgacctgccgtcgaagtcgccctcgatcggacggaggacagatgaccgccaccagcgagaccacccgaggcgtgcccggcctcgccgggcccgcgctcagagccgaggtggagcgcacgatcgacagcttcctgaccgcccggcgggacatgaacaccacccccgagtggcgcgacggggtcgacgccctccgcgccctggcactcgccggaggtaagcgcacccgaccgacgttctgctgcctgggttggcgcgcgttcggcggcggggaccaccccggcatcgtcgtggccgccacggcgctggaactgttccacctcttcgccctggtgcacgacgacatcatggacgacagcgatgtccggcgggggaacccgacgttgcacgtgacgatggccgaggcacaccgccggctcggctggcgtggcaacccggaacactacggccgtaacacggcgatcctctggggcgacctctgcctgacctggtccgacgaggctttccacggctgcggtctggcaccggagcgggtgtcggcggcggccgggttgctccaccgcatgcgcgtcgaggtgctgctcgggcagtacctcgacatccgcggcgaggctgtccgggcagacctggccgactgcctgacgatcctgctctacaagtcggcgaaatacaccgtggagcgcccgctgcagatcggcgcgatgctggccggcgcggcccgacccgacgtggaacgactctccggcttcggcgtcccgatcggtgaggcgttccagctgcgcgatgacgtcctcggtgtcttcggcgacgacgccgtcaccggcaagtcgatcctgaccgacctgcgcagcgggaagtccaccgtactcatggcgctgacccgggagcgggcgaacgccacccaggccgccgagatatcccggtggcacggcaacccgggcctcggcgagcgggaggcacaacggctgcggcacatcgtgcgggacaccggcgccctgcggacggtcgaggagatgatccacgcccgcaccgaagaggcggtggcggtgctcgactccctgcccatcgtcggggaggtgcgggtggcgctgaccgagctggcccacacgctgtcggtccggcgcagctgagagtccaccctgcacgcacgtcccacgaccaggagggcacgggaatgacggttcacgacccccggcgcacggtgctggtgaccggtgccgccgggttcatcgggcgacacctggtcgagcacctgagcagccgggaccgcacggtgatcggtgtcgacctgcgccaggcgccgccccggccggcgaacgcggcagccggccggcacatcgtcgccgacctgcgcaccacggacatccgcccgctgctcgagggggtctcctcggtggtccatctcgccgcgctgcccggggtacggccgtcgtgggatcgcttcgacgactacgtcgaatccaacgtgctcgtcacccggcggctcctggaggcgggtgcgcagaccggtctacgacggctggtcatcgcctcctcgtccagcgtctacggcaaccagcgggacggggcgatgaacgaggaccaaccgcccgccccgatctccccgtacgcggtcaccaaactggccgcggaacacctcgcgctggcctacgcggcccgccctgcctccgtgctgaccaccatggccctgcggttcttcaccgtctacggcccccggcaacggcccgacatgctgatctcgcggatcatctcggcggtccaggacggccgggagatccgggtgttcggcgacggatcgcagcgtcgcgacttcgtcttcgtcaaggacgcggtgcgcgcggtgacggcggccatcgacgcgccgggcccgagccgcgtatgcaatgtcggcaccggtgagcagatatcggtcaccgggatcatcgcgctgatcagtgagctgatgggccgcccggcgaacgtccggaccgaatcccaccgggcaggtgacgtggtgtccacgcgggccgacaccgcccgcacggccgaggcgctcggcttccgcgcgtcggtctccctgcgggaagggctccgggagcacatcgacgcggtacgcagcgagtccatgcccgtcggcgcctgatgggccggggacccgtgctgaccgtcatcgtgccgcacttccgctgcgccacctaccttgcggagtcggtcgcctccgtactcgggcagacccatcgggacctgcggctcgtggtcgccgacgatcactcgccggacgacgactggatcaccgcgctcgcaccgtggcgggacgatccgcggctgacgctgctgcgtgcgtcgcgcaacgtcgggccgctgcggctgaagaacgccgtggtcgagtcggtctccacgccgtacatcggcttccaggatgccgacgacgtgagcgaacccgaccgcttcgcccgccagcttgccgtactggagcggggccgcgccgatctggtcggctgcggctaccgcatcatcgatgcgcggggcaacccgcagggggtacggcacatgccgcgcaacggcaacctggtgcaccggttcggccgcaagacggtcgcgctgcatccggcgacggtcgtgcggcgcgaggtcttcacccgactcaacggcttcgacggcacggcggccttcggttccgacaccgacttccagctgcgggtcgcctggctctaccggattcgcaacctgcgcaggccgctctatcgctaccgcgcctgggcggcctcgctgaccgcagccccggagaccggcttcggatcacccgcccgggacgagtgcatgcggcggatggccgagttggagcagcgccgccggcaggcgcgcagccgcgcggatcggctcccgttgctggtggcaccgcccaacgacgtgtcgttcgcgctgcacgaggtggcgtgagttcgctgcgggcgtggtgcgccacgcccgcagcgtcgcccctaccgcacggccgccctgatctgcatgtgcttgcgccgccgtagccgcacacccgccagcaccgccgtgaccagcatgaggccgacgcagtacagggcaccgtgcagcaggcccttgcgcaggaattccgggtcgccaccgtcgaggccgtagagccccacctgttgagtcgaccagaacggcagggccttgccgagcttctgctccggatcgaccaggaactgcaacccggtgatcgagatcaggacgagcgtgccctccagatcgtgtggggccaccgcgctgatcagcaggcccagcggggcagcaaccacgatcgtgagcccgaactcgatggcgacggcgccgggccgctgcaacgtgtcctggtcgacgtagatgagcacccagtacaccgcggcgagcacggcacccagcgtcagcagcgcggtgagcgtgccgaggtagaggtgcaccggccgatagccgatttggcgtagccgccgctccaccgcctttgtggtgttgctcaggaacaacccggccgtgctcaccgcccaggccagacccaggccggcgaagcgaatcgccttctccggattggcgtcgtgccgggcgatgtagaacagcagtggtacgaacgcgagcagcgcgagggctacccgccgccgggcgaggtcccgacccatcatctccgcaacggtgaaaagcttggtcatgagtcggcggccgccttcgctccgaggtcgagaacggcgtcgacccgatcgagtgaattcaacatgtgtgtgatgaccagggtcgacttgccggcgtcgcgccgttgtaacacccagtcccagaagtcgacgtaactgccccggtcgaaaccctgatagggctcgtcgaggaggaggatgtcggggtcggaaagcgccgcgagaatgaggttgagcttctgctgggtgccaccggacaggtgccgggcctgcgtcgacatgccgccgtcccaggccaggctcttcgcgagctgccggccggtcgcctgggcctcgccccggctcatcccgtgcccggcaccgaacaatgcgaagtgttcgtcggcggtcaggaagcccatgagccctgcctgctggggacagtaggccatgctctcgtccacttccaccacaccgctggacgggctgaccagacccgcacagatcttcatgagcgtcgacttgccgcagccgttggcgccgatgatggcgaccacctcgccagcgcgtacggtgagctcgacacccttcaggacgtgattcttgccgtaccgcttgtgaatgttcgaagcatggagtcgaatcggtgtcgacgagtcaccctccgcgggaccgggcctggtagccgtcacttccgaagcggtgtcactcagatccataggagcaccttagtacacggtcacgctctgctgcgatcacgctcacactggatcaacgtgtcgttcccacggtgtccttttcccgtgagccggccaggctgcccatcagggcgggggagaacgcccgcccacgccacggccgattcggtggatcagacgactgcggggtttcgccggttgccggttgcgggtagcgcatggaccgtgaaccgcaatctcgtcacagcgttacaggctgcctggggactaccggagttccgcaacgggctgcgtcacatcatcgacctcgacgaggtggcggtgctcctggccacgctctccgtcccggaccacgaccacgaggtggaccgcagcctgctcagactgctgcggtccagcctggagatgggcgagatccgggaagcggtgttgctgctgctggagcaggacgaggtacgccgaccgctcgtcgccgcggtggtgccaccccgggccgaccgaccgggccaggctgccgccatcaccgcggcgtccgaggaccccgcagtgcggcgtgaggtacggacggtgctggacgacgcccgggtgcgcgcactgctctggcaggcgatcgaggcccaggtgaacgacgaccggggcggcctggtccgccgcgccgcggtgctgttcatacggcatccgagcgtgccccgcctcgcctgggccctgcaccgccacggcgtgctccgcgagctacgccgcccctccacgtcggccccggcggagggcaacggacagccgcgggcgtaggtgaacctcacgttgcccgacgaccggccggcgcacgcggggccgcagcggccccgacgagctggtcctacactcctgccatgttcgagtaccacgggtggatcaccctccgctcgacagcggatgcggtcgacgacgacccaccccttcgcctcgacgagatccaggacctggtcaacagcctcgccgggtacgggctgatggaccttcgcccggccaacggcgcacaccacgtacacctcggcggtttccccaaccatcggggcacccggggtgccgacgttgtcgagctgttcaccaggatcggtcatctcgcccccgggtcgtacgggctactgtacgtccacgacgacgaggatcccgggcacgaactggtgttccgtgtccaccggctggtccgcggaaccgtctccgaacacaccgaccacctgctctcgccggtgatccccaccctcgaagacgagtacctcgactgatccgggagaaccagccggagaggctctcgcgccacgctccgcccgcgacggcggacgtacgcgatgatggggtttcgtccggagggtgctgatgggaaaaccgacgctgttcctgaccgtgggactcccctgcaccgggaagaccacggctgctcggcgcatcgagatcgagcggaaagcccttcgtctgacgaaggacgagtgggtgaaggcgctctacggtcccgagaatccgccgttggcgggggaagtgatcgaggggcggctcatccagatcgggctgcgcgctctcgcgctcggcaccaatgtcgtgatcgactacggcctgtgggaccgcaacgagcgttccgccctgcgccaggccgcggtggacctcggcgcgagggcggagttgcgctacttcgcactgaccccttccgcgcagcggagacgacgtgaccagcgccaggccgaggcgccacacaccacctggcccatgtccgacgcggaactcgtcgagtgggcaacccgcatggacatccccacgccgggtgaactcgacggcagcgagcccatcgacggcccacctgcgggcttcacgacgtgggacgagtggcgcagccatcgttggccgccgtcggtccactgagacctgccggtcggacacccgcggtacggcgatcggtaccggcatccccaccagcccgctgaccgggtcgcacctgcactgacgctcgccaatgttgcagagtgcatccagacgtgttggaatggctcatatcgccgcagcgcgggggcaaccgtgctgagaacggccagcacgggagcaccacggtgtcggcagcgccccgccttgacggacgcccgcacgccgcgccgccgggccatcgccctgctggcgggaaccatgccgggcaggcactcccgtccgccatacgcacccctggcacgcgccccgacacggccgacgcgccgacgcctgacagcggggcgtgcgattcggtctgccagcgcaccacaaccggcgacgaggtgagaagcgatgatttccggacggagtttcgcggggtggtaccgccaccgcctggtgtgggtggcggtgatcctgctggcggtgtcggccggcacggtcgtactggccaaccgggccgatccgaaggcggagccggcggatctgcaggcgcagatcgtggcccggatgcgcaccacgctcgaacaggcggacccggggcagcaccagcacgcgggccacggcaccccgcagggtgccggcacggagaagccaccggtgatctgcggggtccgcgtctacggcttcgagccggcggaggccaccgcgttcgccgacgtccggacgatctacggctttcacctctgcggaatcgccgagcagaagcgcccctgggacgtggcggtcaagctggccggcccattgatcatggacatgtccaccgatcccccgggcatccaggtcgtcgaggcgactgccgaggtcaggttcgtcgaccggctgcgggagatgttcccgcccgcgtacgcgacgatggcgcagcaggaggctttgggcgcctcggagatggccgacctgcgtcgccggtacgacaccgcagccaagtcatgagccggtccgacggccgacccggcgtcgcacccggccaggtcgaccatcgggccgaatcccccggccagccgaccggacctgcggcgatgctgcggccgtgaccccgcacgcgcgttgttgatccggcaatctcggacacctcgttccacatccgacagctcgatccaatccgactaaatatcgatcaacattgatatttggcctcatcggtgccacagtggattagggagcattagcaacccgatgtcccgtaccgacgaatggaggatgaacagtgcgttcacacaggtccgaccaatccggcggacggccccggtccaccgcccgccgatccctccaggcgctcgtcctgatgttcgccatggtggtcggtacgctgcccgtgaccagcgcagcccaggcgcacggcaccatcatcaatccggcgacccgcgcctaccagtgctggaatacctggggcagccaacacaccaaccccgccatgcagcagcaggacccgatgtgctggcaggcgttccaggccaacccggacaccatgtggaactggatgagccaactgcgcgacggcctcgccggccagtaccagcccagcaccccggacgggcagctgtgcagcaacgccctgtcccggaacaactcgctgaaccagcccggggcgtggaaggcgaccaccgtcagccgcaacctcacggtccagatgtacgaccaggccagccacggtgctgacttcttccgggtctacgtcaccaagcaggggttcaaccccgccacccagaaggtcgggtggggcaacctcgacctgatcaccacgaccggacggtacgcgccggcacagaacatctcgttcaacgtctcgatcccgtcctcgcgcagcggaagccacgtgctgttcttcatctggaaggcgtcccacgccgaccagacctacatgtggtgcagcgacatcaagatctgatcggcgacacggcaccactccggcccaggacgttgacgtcctgggccggatctcactctcacgggacgcggctggtcaacgtgcccggtgatgggttcgacgcggtcgtcgccgcacggagagcgccttggcggggtccacggtgctcccggaaggcggctgctccgggcacctcgtaggccccccaaccagccggccccggcacgggcgcgtcgggcctccgggcaggtgcgcggtggtcgtccgggcccacgccgatcgtcggctcagctcagcgagcaggatgtcccgttcagggtgaacgaggacggactgccggtgcctccggtgtgttcggccacgaacccgatggcgatcgacgcgcccggcccgatgatgacgtggtcgtccaggccacgcgccgtgacgttgccactggatggcgagtacaccgcgttccagccggtggtgatgcgttgtccggcgggcagcgggaacgccagcctccacgaggtgatcgtggtgacgccggtgttggtgatggtgatactgctggtcagaccggtgtcccaggtgtagaccgagtaccggacggtgcacgcggccccggtcggtggcgggctgctcggtggcgggctgctgggcagggcggtgctcggcggcggagcggtgctgggggatggggtcatgccggccggcgccgtggtcgacgcgctggcgccgtcgatggcggcggccagcgtcacggccgccgcagcgacaccgagaaggctgctcttcagtctcatggcggcggcgattcctccggaggcgggcgcacagcggcgcggcggagcggggccccgtcggcacggatcgacccggcgcacctcgcggcacccacattaattgatcaacgtcaattgtcaaacgaccaccagccccggccccggccaggccgacgccccgaacgcatccacgcctgtctcttttgccggtcctggcgtcatctgacggcaggccccccgacggtatttcccggcgcactcacggctcttccactatgtcatcgcgccgacacggcatcgcaacacgccggactcggcgtcccccgccgatgggagaaaacctgtcatgggcaaggtaggaatgcgcgtccgccgctcgacgctggccgtactcacgggcgcggcgctggccgggctgctggcggtcaccgcagccgccgcggcacccggcgaggcaccgcagggcaaggccgccgcggcggcccgtaccgacgtctatctcaaggacgtcgccgccgacaccggcgtccaaccacactcgtacggcccgctgtgggccagccccgacatcaaggtctgcccgaccgccgtcgaatgtgccgtcagtcagaacccgatcgtcggcgtcaccaactacctcttcctgaaggtgcgcaacgccggcccgtacggctcgggcatcgacaacggagtgctgcgggtctaccggaccaccccgggcggcggcgccacctggccgacgcactggacccagatcggcgcggccagcgtgcccgtctacccgggcgtcaccacggtcacgatcccgtggaccggggtgcccggaccgggccacttctgcctgttggcacgctgggtgtcccccaccgacccgatgaacttcgagggcccgagcaccagcgtcaacacccagtacaacaacaacatcgcctggcgtaacgtcgactcggtccggctcacgccgggcgggccacgggtcgtccggccgttcgccatcggcaacgtgaccgccgcggagaccaagaacgacctggtcttcacccagccggcagagcaggcccggggtgcccgggtcttcgtcgacctcggcccgaccctgttcgcgcgctggcagtccaccggcatggtcggtacgggaatccgccaggtcggcaagaccgagttggagatcgtggaaccgacgaaggccagcctcagcgggctggtgttgaagcccggtgagcgcctcgagttctccctgtccttctcggccgacaccgccctggccaaggagaccgcgttcaacgtgacccagttcgggccggacgccgacggcactgcgcgcgccgacctcggtggggtgcagtacctgctggcgggcgagcagcgggggtaacgcgggtcgtcgccacctccgggtggggcgcgggccatccggcccgcgccccaccttcgtcggtcgggccgaggccggccgccgggtgcgcgtcgccggccgcaccgtcgcccggggggcgggccgccggtcagcgcagacagacccaggcggtggacaccgcaaacccgacggtacgcgcgcacaactggaccagccccggcggcgggacgcccaacgagaagaatcccaccgcgtccaccggtgcctcgtcgtaggtgccggaggccgtgcgggcactcacccgtccaccaccggacggggagagctgcccgacgatcccggtgtcgctgacctcgatctccaccaccaccgacccgctgcggaaggacagtgtccgcacgggcgcggcggaccgggtcatccccaccaactcggcgtcgatggccgagtcgaacgtcagctcggcgacggccagttccgcgtccgcggtacgccaggtgaacgctgccaacgccgcgctcaggtacgcctccgagaccggcccggtgtcccacaacgccgcgcccagctccgtggtcagtgcatcgtcatcgaccggcggccacggctccatccggttcccccccgttcgtaccgatggtggacgctccgagaaactcgacgatcgccggacatgcccgcagcctacgcaggcaacgggcctgggtggggccgatgctgccgaccggcacgcccaaccgctcggcgacctcccggtagctcatcggcgggtcggccgtcagcagcgccaacaacgcctggcagcgtggcgggagctgcacgaaaccctcgcgcaacgcctgtcgccgctccgcgcgcagcaactcgtcgtccggggccggagccatcgcggccaactcccgggagaacccgtggtacggctcaagcgactggtcgtacgggtcgacgggttgggcccggcggctgaggcgggacaaccggtagcactcgcgccgggtcgtggtggccagccagcccggcagcgcctcaggctcgcgtacccggcccaactgctcgaccaggcggagccagacggtctggttgacatcggccgcatcggccctgctgaggtcgtacgcccggatgaccgagtacacgatcggggtgtaccggtgcaccagttcggcccaggccgactcgtccccgccggcggcggcggacagcagggtggtcgcgctggcaccggtcatcgggtcaccgcccgactgctgacccggctcatccgacgagcaccccgtagtcgggatgccagcggggagcggcgagcagggtggtgcgcgcctggtccgcgttgagccctccggcggtcatcagagcggcgagccgcccggagacgtgggcggtggcgaacgaggtgccggcccagatggcgaacccgtggaactgccgtaccggtcgcccgggcagcggcagctgtccctcgacgtaggtgctgtggcgctcacccagggcgcaggcgtccacccagggcccgtagcaactgtcgatcgccggcaccagaccgccggtcgtctggctgaccgccgcgaccgccagcacccggtccagggcggcaggccagagcggacgactggtgccggcgttgccggccgccgcgaccacggcgctcgtcgccggcagcgccgccagcgcgttggccagtggcagcgatggttgatcgtccagggtgtatccgcccagcgagaggttgaacacggaaacctgcggcgcgagccggctcagggcggccaccaccgtcgcctcgtcccccaccccggtcgggtccagggcctgctccgggtcgacgcgtacgcccggcgcggcctgccggaccactcccgcgacgaaggtgccgtgcccgccctgcgaggcgagcacgtcggtgtggacgtagagcgggtcctcgtcgtcgagctgcggcagatatgtgccgccgagccactgggggtgacaggtgccggcctgccgccagattccggtgtcgcagatgccgacggtgacacctgcgccctcgccgaggcgggtcgggtcgggcggcggcagcgccgcagccgagcggggtggggtgcccgggtttcccatgatgttgcccaggccgatcagcacgtggtgcggctgcaccgccggcaccggctggccgggccactgtcgggggtcacgcagctttcccacgacgacggggatgtcggtttcccgatcgaagagcagccgggtgacaccggcgaagccggctccgacggtcacgggataatcgtggttacgcaaccaggtggcaacccgtttgacgtcgacgtcggcggcgagcagttgccggggccggtagaggatctcccggcccggcgcggtgtaccgccggacgttctggtcggctgccagcgccgcttcgaacgccttctggtaggcgtcggcatccgtgtcgccaccggcagccggggtggggcgtggcacgacgccgaacgggaccgccgctgccagtgcggacagcgccaggatccgccgtcgggacagccggccgcccggacccggcacggtggggtggtcagacggcggcacggatcctccatgctcttgagagaagcgcgcccaccgatggcgggcgcggcgaattccccgaccgccaattattccggaccatcgccggttgtcaattgcgcaccaagcaggacacccattgacagtattcccgcgacggattttcgacgttgactcgcggcggtccgtcgtggactctctaggcatgcccgacagcaccggttccggtgccgcggcggcccaggcggcgctcgacgccgtccagcgctacccacccgaggcgatcacggtcgcccaccaggtgctcgccagccagaccgccgacgacgacgagcgctccaccgccgagcgggccatcggcctggccctgcgcgagctcaacgacctgaccggcgcactgcgccacctccgccgggcggtacgggccgccggcacaccccgtacccaggcactggcccggatgagcctcgggtacgtgctggccaatgccggtcgtaccgctgcggcgctgcgggcggtgaccgccgcgctgccccagctcaccggtgccgacgccggccgcgcccggatgcagcgcggggtggtgctgcactaccgcggacgctatgccgaggcggtgcgtgactacggcatcgccatcgacatcgcccaacgcgaggacgacccgttgctggaggcccgcgcccgcaacaaccgggggctgctcaacgcacaccggggtgcctccggcggcaccgacgacgacctctcccgggcggcggcggtcttccgccggctcggactcgacctcgcggcggcggacgcccgctggaacagcggcatcgccgccgggcaacgcggcgagatcgccgtggcgctgcgttgcttcgccgacgtcgacgaggagtaccggcggctcgccgttccccgtccggcgctgctgctggaccggttcgagctgctgctgtcggtgccgctgctcgacgaggcggtcgaggtcgccgccgccgccgtacgggagctgcaccgccggggaatggcctcggacctcgccgaggcgctgctcgcccaggcgcgagccgcgctgctggccaacgacctggacaccgcgaccgcggcggccgcccaggcccgggtcaggtttcggcgtcagggccgccgcatctgggccgcgttcgcccggcacgtcgaactgcgcgcggagttccgccggggcacccgcacggcgaccctgctcgccgcgatggtgcgcacggccggccagctcgacgccaccgggtgggcgggtccggcgctgaccacccgcatcgaagccggtctgctggccgccgcctcgggtcgcgtcgaccgggccg

SEQ ID NO: 2 - DNA, 1695 bp, synthetic construct
  misc_feature 1..1695: note = P08-G05-PacBio_cluster016 - ctg4_242
  source 1..1695: mol_type = other DNA
gtgagcaagatcagtcccggcaccgccatcctgcgcagggcggtcgcccgcaacgggcgacgcctctggggtggaacggcgctggccggcgtctatcagctctgcgcggccctggtgccgattctcatcggtgtcatcgtcgaccgggccgtcgccaccggcgacctcaccgcgctgctcacctggatcacgaccctggcgatggtctacctcgtcctggcggtgacgtaccgcctcggcgcgcggcaactgcaacgcgccatcgccgaggagggccaccggctccgggtggaactggcggcgaggatcctggacccgcgcgtgctccgcaccgaacggcacaccggcgacctgctctcggtcaccaccaccgatgccgagtacacctcctacctgctcgaccacattccacggatcaccagcgcgctggtggcggtcaccgtcagcgcggtcgcgctgctgctgatctcgccgccgctcggactcaccgtgctggtcgccatcccgatcgtgctggcgatcctcaacctcaccgcaccgctgatcgcccgacgcgtcaaggaacagcaggaccaggccggccgggccaccgcgctcgccacggacctggtgaccgggctgcgcccgttgcgcggcatcggcgcccaggacgccggcgccgcgcgctaccgccgggtcagccgccgatccctggcggccgccctgcgcgcctcccggacccagaacgcgtacctgagcgcgtccacgatgtcgagcaccctgctggccgggggcatcgccgtcatggcgggctggttcgcgctcaccggtcgcatcacggtcggccagttcatcacggtgatcggctccgcgcagtttctcgtcgaaccgttcggcgtgctcgcggtcgtacccagttggaccgccgccgcccgcgcgtccgcggaccgggtcgccgccgtcctccaggccggggtggtgctgccgccgggcgtcgccgcgccccccgacaccgggtacgagctgcgcctgaccgacgtgacgtacggcccgctggccggcctccacctgcacgtccgcgtcggagagttcgtcggtgtggtggcgcgccggccggcggacgccgaggcactggcgcggctactggcggtgcccgacgcctaccagggggacatcctgctcggcggagaacggctggagaccgtggagcgggaccgggcgcgccgcgtgctgcacagcgaaccgcaccgtaccgatctgttcaccggcaccctcgcctcgaacgtcgccatccacgacgacgacggcggacggctggccgcggccctgcgcgccgctgccgccgacgaggtggcacacctgcatccggcgggcctgcacatgccggtcaccgagcgcggcgcgaacctctccggcggccagcgccagcgtgtcgcgctcgcccgcgcgttgctcgccaggccgccgctgctggtcctgcacgacccgaccaccgccgtcgacgcggtcaccgaacaggccatcgccgacggcatccgggcgctgcggcacggaccggacgccagctacggcaccgtcctcatcaccagcagcccgacgctgctggccgccaccgaccgggtggtgttcctcggggacggcaccgtcaccgccgagggtgcccacgccgaactcgtcacccgcgacgacagctaccgccgggcggtgcggcgatga

SEQ ID NO: 3 - DNA, 1860 bp, synthetic construct
  misc_feature 1..1860: note = P08-G05-PacBio_cluster016 - ctg4_243
  source 1..1860: mol_type = other DNA
atgacgcacccgaccctgcccgtggccagcccgcgcgagagcagggcgtggctcggtgcgcaactgcgcgcccgcgcggtggccaccggcgccaccctggtcgtcgggctgctcgccgccgccaccgcgatcgttcccgcctacgccctcggcctgctggtcgaccgggtccgggccggtgccggcaccgacgcgctcgtgccggtcgccgtggtcatcggggccgccgcagtcgtcggtggtctggtcgccggcgtcagcgcccacctgatccgggggttgggtgcacggatcctcgccgacctgcgtgaacgcgccctggacgccgcgttgcgcctgcccgtggcggtgctcgaccgggtgggccggggcgacctggtctcgcgggtcggcgcggacatcagcgccatcgaccgggccgtctccgaggtcctccccaccatgatctcggcactgctgctggcggcgatcagcctcggcacgatcatcggcatcgactggcggctggggctggccggcgcggtcgccgtcccgctgtacgtgctggggttgcggtggtacctcccccgggccgcaccggcctacgcggccgagcgggtcgccatcgcacagcggtcccagcagttcgtcgagagcgtgcagggcctgcggacggtgcacgcctaccggtgggaggatcggcacctcgccggcatcgcggctgcctccgcccgtgcccgggacatctcgacgagcgtgttcgccctctacacccgtttcgtggggcgcatcaacggggcggagctggccgggctcggcgcggtgctggtggtgggattcctgctcgtacgccgggacgctgtcaccctcggccaggtctcggcggccgcgctgctgttgcaccgcctgttcggtccggtccttcagctgatggtcaccttcgacaaggcccaggacgcgggggccagcctcgcccgcctcgtcggggtgctccgcaccgaccccgaacgccgcgcgggcggcaccacgcccccggcgcacgacctcgtgctgaccgacgtcggcttcagctacgacgggaccacaccggtgctgcgcgacatctccctgcgggtcgggccgggccaactggtcgcgctggtcggcgccaccggcgccgggaagaccaccgtcgcatcgatcgccgccgggatcctgcgccccgagcacggggcggccaccctgggcggcgttcccgtcaccgagctgcccgcccggacgatcgccctggtcagtcaggagacccacgtgttcgccggtccgctcggcgaggacctgctgctggcccgcccgcacgcctcgccggaggagctcaccgcggccctggcccgggtcggcgccctgtcctgggcgctcgccctccccgacggcctggacaccgtggtcggcgagggcggccaccctctgaccgcggcgcaggcgcagcagttggcgctcgcccgggtggtgctgctcgaccccgcggtcgtggtgctggacgaggcgaccgccgaagccggcagcgcgggggcacgcgacctcgaacaggcggccgccgcggcgctggagggacgcaccgccctggtcgtcgcccaccgcctcacccaggccgtgaccgcggatcacatcatcgtcatggaacagggcaggatcgtcgagagcggcccgcacgccgacctggtcggcgcgggtgggaactacgcccggctgtgggcggcgtgggccgaccgcgccaccgccgacccggggccgacccgcaccggggccgcagcggggcgcgcggtggacagcggcggcccgtcgacaccgcaaccgcggcacgggcggtggaccggaccgccgctgaccgctcagcgggagtag

SEQ ID NO: 4 - DNA, 609 bp, synthetic construct
  misc_feature 1..609: note = P08-G05-PacBio_cluster016 - ctg4_244
  source 1..609: mol_type = other DNA
gtgaaccacccccgacccaaggcccggctctcccgcgcgctcggcatcccattgacccgcaagtgcgtgcggtacttcgagcggcgtccctacccgccgggcgtgcacggccgcaaccgtcgcaagacctccgactaccaggtgcggctgctggagaagcagcggctgcgccaccagtacaacgtcagcgaggcccagctgcgccggacgttcgacgaggccgcccggggcgccgggaagaccggcgagtcgctggtcgcgctcctggaacggcgactcgacgcgctggtgctccgggccgggctggcccgcagcatctaccaggcgcgccagctcgtcgggcacggccacttcaccgtggacggccgcaaggtcgaccggccctcgtaccggctgcggcccggccaggtggtcgcggtgcgcgagcgcagccgtgccctgccaccgttccagctcgccgccgccggcgcgcacgccgacgaccgaccccggccgtacctgtcggtggagctgaccgggttgagtgccacgctgctgcgcgagcccgcccggcacgaggtgccggtgatctgcgacgagcaactcgtggtggagttctactcccgctga

SEQ ID NO: 5 - DNA, 735 bp, synthetic construct
  misc_feature 1..735: note = P08-G05-PacBio_cluster016 - ctg4_245
  source 1..735: mol_type = other DNA
atgcgacccagcccggcggagatcgtgcgtaccctcgtcgcgggccggctgcccgccctcgcccacctggccggcagcaccggcccgcaccaggtccggcacgccaccgacccggacggccgggtgctgctgctggtgccggcgctcagcgacctcgccgcggcgctcgcgccgccggccggggacaccgacgtcgccaccgccctcgacgtgctcgacctgccgcccaccgccggcgccccctcgctcggccgggcctgggtctccggctgggccgaacgcctcgacggcgacgaggcacgtcaggccgcgatggacttcgccaccacccagccgaccggcgacctgctagacgtgggcagccggttccagctgcaccggttcgaggtggccgaggcccgctgggaacacaccggcgggatacgcccgatcgacccgcaggcgtacgcgcaggccgagcccgacccgctgcacgcggccgaggcgggattgctggccgacctcgccgaccaccacgccgagcaggtcgcgggccacctgcgccggcagttgaagctctccggtgccgagtcgccacgggtggtccggatcgaccggtacggcctgctggtcgcgtacggccggcccggtgcgacccgacgggcgagggtgagcttcgcgggacccgtcgccgacgtcgccgagttgtcccggctgctgcacccgatgctctgcccccggcaggcggccgcctga

SEQ ID NO: 6 - DNA, 1302 bp, synthetic construct
  misc_feature 1..1302: note = P08-G05-PacBio_cluster016 - ctg4_246
  source 1..1302: mol_type = other DNA
atgacgccacatcaaccgctcgcccggctcctggccgggaagctggtcgatccggcacccgagggatggcagcgggacttcggccggctgcgctgccaggagccacaggcggtcgtgttcgccgccagggtcgccgacgtgcaggagacggtgcgctacgcgctcgtcggcggcctgccggtcgtcgcacggggcaacgggtgcagcagcaacgggcagagcctcaccgacggcctgcagttggacaccgccgcgttggaccggatcgaactggtcggggcggcgacggtgcgcgtcgaggccggggtgcgctggggaacactgcaccgctggctgcacgagcacggcctcgcgctgccggtcgtgaccagcaacgagcacgcctcggtcggcgggacgctgtcggtcggcggcttcggcccgacgtcacccttcgtcggcgcggtcgtcgatcaggtggtggccctggaggtcgtcacgggtgacggcgcactcgtgcacgcgaccgccgacggcccgcaccgggagctgttctcgtacgcgctctgcggtctcggtcaggtgggtctgatcgtccgggccgagctgcgggtggtcccggcggaaccggccgtcctggtccgccggctagacggcgacgacctcgccacggtggcggagcaggtgtgggctgacgagcggctgcgtctgtgccgcgcggtgtactccaccgccgcgcgccggtggaaggtcgaggtcggccggtcggcgcgcgacgcggcggccgagtcggacgagaccctggtcgccgactatcaccagcggctgttcgaggcggaggcgggcttctccgtcggcatcgcccgtggccaggttgccgcgggtctgctgacggacgagaccgaggcgcgccggctgtggagcgacttcttcttgccgatggagaaggccgacgccttcttcgccggggtccgcgaccggttcgccgatcccgccgtggtcccggtggtcaacggcgcattcctgcgcgccggggaccggccccggccgccgttgagtccgctgcccgacgccgaccgggtacgcacgttcggcttctactgcatcgtgccgccggaccgggtcgacgagtaccgggagcatttcgacgctgccggcgcgctctgtcacgagctcggcggccggcagtacctgcacggctatcaccgggtggagcgggacttctaccgggcccagttcggtgggcccaccgtcgagcggtggcgggaggtcaagcgccgctacgacccgggcgccgtgctcggcccgcagctcccgatctga

SEQ ID NO: 7 - DNA, 810 bp, synthetic construct
  misc_feature 1..810: note = P08-G05-PacBio_cluster016 - ctg4_247
  source 1..810: mol_type = other DNA
gtgttgacgtttcctgagttcaaggccgagatggacgacagccaggcgcgggtggtccaacggtgtctcgccgagctgaccagtctgggcatgtcccggcagggactggacttcgtgcgtgaccttcccggctacccgcgtacgccgtactacacgacgctctgggtgcgcgacccgcgggtgcgcgccgacctcggcgccggggtctcgatccacgcggtcggcgtgaagctgatcgacgacctcatcgacgacgaccagccgctctcgccgcgcgaccagatcttcggggtccaactgctctcggtcggcaccacactgctggccagccagcagcggccggtcgaggtgctcaggacgctcgaggacgactaccgcgacatctggcggcaggagatcgtggaggtcagcgcgccccgaccgaccacggtcgagtcctggatcgcggcggcccggatcaaggccggggtgatgctcgccaactacgccgcggtcaactgcctggccggtggggttcccgagatggtgacggcggcccgggagttcggtgaggcgctgggcgtgctgtacatgatcggcgacgacgcggtcgacttcgacgagatcgacgagcgcgacggcaacctcgcccacctggtcgcctcgggcgcggccgacctggccgacgtcgccgccgcggtcgacgagtggcgcgaccgggccgtcgcggcggtgcggcagggcgacccggtctgcgacctggtgccgttcttcgaccacttcgcggcgaagctcaagggcctgatcagccggccgacggcgaccgtctga

SEQ ID NO: 8 - DNA, 816 bp, synthetic construct
  misc_feature 1..816: note = P08-G05-PacBio_cluster016 - ctg4_248
  source 1..816: mol_type = other DNA
gtgctctccggagtgagcggtctggacgacgagtgggcggtcgcgcagcgcgtcctgctggagcgggtcaccgaccggctcgccgggatcggcatgaccccggacggggtggagtacgtccgccagatgaagggctatccgcggattccctacttcgcctcgctctgggtgccggacccggtgcagcgccaggagttggggatcgcgatctgcacccacgcgatggggatcaaactggtcgacgacctgatggacggcgaccaaccgatccggccctgggaccagatcctcggcatgttcctcacccagagcagcacggcgaccttcggggcccaccaccggccggccgaggtgctgcgcgtcttcgacgaggactacgaggtgatctggcgggagcagatcaacgaggcggaacgtccggcccacgacctggacagctggatccactacgcccgcatcaagtcgggccggatgatgatgtgttacgcctccgccgcctgcctcgccgccgatgtcgtggagcggctgcccgccgcgcgcgcgttctgcgaggcgttcggtgtcctgttcatgatcggcgacgacatccgtgaccaccgcgacctcggcgaggccggcggaaacctgtgccacctgatccggagcgggcaggtgagccgggacgcggccgtcgcggcgacccgccggtggcgcgcggaggcgctgcgcgccctggcggagttcccgccggagttcccgatcgccggcttcatggccgacttcgccgaccgcctgatcacctttctcctggaggacgaccgtgttgacgtttcctga

SEQ ID NO: 9 - DNA, 1266 bp, synthetic construct
  misc_feature 1..1266: note = P08-G05-PacBio_cluster016 - ctg4_249
  source 1..1266: mol_type = other DNA
atgacccgcagcgtgcaggtcatctcgatgcgcacctggggtgagctggggaatctcctcgccggccggacgctcgccgcgaccctggccgccggtctggacgatgcggaggtcaccgtcgtcgaggcggagacgctcttcccgcagttcgccgaggccggtgccgccattcgtgagctggtggcggcccggctgcccgccgacgaggtgtgggccggatacctgcgcctcatgacggacttcaccgaccggctaccggcgggcctggacgacgacccgctgcccgcggcgaccgtcgatcggctggcacccgtcgccgaccacttcgcccagacgaaaccggacctggtgatcggcaccaagggggtgatctcccggctgagccatgccgcgctgcggatcgcggacctgccgaccccggtggtcaactacgtgaccaacgaggggctgctgcggctgccggtgcaccgggcgccccacctgccggtccacctggtgcagttcgaccgggccaaggactatctcgtccgcgagcacggctaccggcccgagcgggtcgtgccggtcggccggttgctcgcccaggcccaggccgcccaggtcttcgagggcgccgccgcaggcatcggggacgccgacgccgtcggtgccgccgatccggcgttgacggcggccggcacccggatcctgatcctgtccaaccggggcggccgggagtatctcgccgccctggagcggatcgcccgcgcgcatcccgaggcggcgctggtcttcatcgcgtacaacaacccggcgctggccgccgaggcggccgaactgctgaccgcgctgggccggtcggcctggcagaccttcgaccggctggcgcagggggactacctgcggtacctggcctggctccacgccgccgacgcgccgctgttcatctccaagaccgggccgaactccatgttggaggggctctacttcggcgtcccgcaactgctggtgcgctccggcctgccgatggaggagtgggtcggcccgttcctcgccgaacacggcgtcggccggggcttcgaccggatggaggagttggtcgaggtcgccgtccgctgggtgggcgacccggatgagctgccccgtcgccgccgccgggcgatggccctcgcccgtacgctgctcgacccggaccgggcccggcggcagaccgtcgccgcggtcgccgcagtgctcgacggggtgccgctgcggatcgacgaggaggaaacgtgctctccggagtga

SEQ ID NO: 10 - DNA, 2130 bp, synthetic construct
  misc_feature 1..2130: note = P08-G05-PacBio_cluster016 - ctg4_250
  source 1..2130: mol_type = other DNA
atgcgcgaagtgcgacttcccgggaatctgagcacggtaccggcgaccgcgccggtgctcacagcgctgcgggcgcaggcccccgggctgctgcacgtcgtggacccgtacaaggtgccgttggccgaggcggtcgagaagtcgcgggcggtggccgacctcggccatcccgcgctgctcgtcggcagcaccgacaacgccgcgttcgaccgacaggtggtcccctacgtcgccgaactgcgcgagcggaccgacctgcccctcgtcctgcacttcccgtcgatacccggccggggcgctccgctctgcccgggcgccgccgcggtgctggtgcacgcggtaccgaactcggtgcggtccttcttcgtctccggtggcctgaccgcgaccgtccgcgccgcgacggcgctcgggcaggacgggcccgagctgctgcgatcggcgtcgttcgccttcggtagcgacggcaagacccacgaggcggtcggtgcgccgccgacggtgcaggacggcgcggaactgccccggctggtggcggcggtcgctgccgccgacttcgacctcgcctacctgctctaccggcacggccgggtccccgaggcggtgctgcgggcctaccggcgtggcctggcaccccagcagttgctgttcgtcggcggtggcgtgacctcgtaccgtcaggtccgggagtatcacgactgcggcgtcgactgggtcgtcgtcggcaacggcctggagaccgccgactggcgggagaccctgcgcaggttgtccggccggccgggcgaccgggccggtgaccccgctgcccccccgacactcgccgccgagttgcagcgcgggctgcgccggcccgtgcgcaccgatctgcccggccggcaggcgtacgccacgaacgtgggccgtctgcacgagctgctgccggccgtggtggccccggtgacctcggcggaggaggtcgcgcacgtcctgcggaccacccgcgccgccgggacgcccgtcgcggtgcgcggcagcggacactccttcggcctgcagggcctgtccgacggtggcgtgctgatcgtcggcgagccgggggaccccggggttcggctgcgcgacgaaggcaccgccgaggtgtccgcccgggcaccctggtacgccgccgagcaggccctgaacgcccaggggcgcagcatcccggtgctgaccaaccatctgacggtctcgatcggtggcacgctgtcggtcggcgggtacggcgagggcacgatcaggtacggcggccaggtcgacctggtcgagcggttccggctgatcctgcccgacgggcaggcccggtggtgctcgcccgtcgagtcgcccgagctgttccggtacggcatcggctcgctcggccagttgggcgtgttggaccgggtggtcctgcgtacggtcgactaccagcggcacaccaccatcgcgacgcggcggcacccctcactgaccgccctggtcggcagcatcgggtggctggccgaggagcagcggtcgccggtcgacctcttctccgcccagcactacgagggcggcttcatcgccacgtacgggatgcggggcgacggcccactgccgccgcaactgcgccgacagaccgagacccgcgtcgaggccgactggccgttgcggtccatgcgggagtcccggcgcaatccggccgacccgtcggtgcacttcatctgggccgactactgcctgtcgctgccggcggcagccgagttcgcccggttcctcgacgcggagatcctgccgagccccggctaccgcgagggtgcgggccggctgctcatgctcggcgtacaccgcccggagcaacgtccgggggtgccgttcaccccggcggccggactacggggcctggccgtcggcttcgggctgtacttcagcgtcccgcgcggggacggggccggcgtcgcacgggtgcaggggctgcaccgtcaggtgctggccgagtgcctcgcgctgggcggccggccctacctggccggctggtacgacctggacgaggcggacctgcagtcgatgtacggcgccgacctcgccgccgtacgccggttgcgccgggaactcgacccgcacggcctgttcaacccgggactgctggcatga

SEQ ID NO: 11 - DNA, 837 bp, synthetic construct
  misc_feature 1..837: note = P08-G05-PacBio_cluster016 - ctg4_251
  source 1..837: mol_type = other DNA
atgacagacatcgcgaagtacatcaagcaacgcgcgcagggcaaaggtgtcctcaacgtgctcagcggagtgtcccgcacctacttccacggcaccggaacaaaggcgctcagcgacgccgcctggctcaagcagaagtccagccgtacgccaccgcagttcagcatgatcgcgatcgagacgatgagcggctgcaactaccgctgctcgttctgccccatcggcaccctcaagatgccgtccggccagatgtcgatggagctctacgagtcgctgctcgaccagctcgccgacttcaccggcgtcattcacccgtacctgttcaacgagccgctgctggaccggcggctgcccgcgatggtgcggatggccaaggagcgcaccaaggcgtcgatctccatccagaccaatggctccaagctcaccgaggaactggccgacgagctgacccggtacgccaccgtcgtcgtcaacgactacaccaccgacctgtcggtcctggaaaaggtccgccggtatccggcccggcccgagctggtgctggtcgaccgcgatccggacgccaccctcaccaaccgcgccggcaacgtgctgggccgtcccaccgtgctgctcaaccagttctgcgtacggccgttcaacgagttctacatcacccaggacggtcgggtggtcctgtgctgccaggactggagcgtgcaggaggtcatgggcaacgtcaacgacaacaccatcgaggagatctggaacaacgagaagtacaccgcccgccgcgcgagcctgctgcgaaaggaacggaccgacctatgcgcgaagtgcgacttcccgggaatctga

SEQ ID NO: 12 - DNA, 1197 bp, synthetic construct
  misc_feature 1..1197: note = P08-G05-PacBio_cluster016 - ctg4_252
  source 1..1197: mol_type = other DNA
atgctcgagcttctgacccggcccgtccggtgggcgatccggcgcggctaccaacccgccgacggcgacccaccacccacggagtgcatcgtggttcgtcccggcccggccggcgtccgggccgcgacggcggtcccggcggtcgccgcggaactcggcgacggcttcggcggcgggctggcgttggtcctgccgacctacgtgggccgggcgccgctgtcgagccaccgcgagaccgtcgaggaactcgtcacccaactcaccgacctcccaccgacggtggccgcccgcccgctggtgctggtcgtcggcatgcagtggcgcgacgaccagcacgacgaggcgatcgcgcggctggggcagctgagcgagatcgccgcctcccggggcgggctcaccttcgtcgggatcgcgatgccgcaggccgggaagagtcgcatgctcaacgccgcgttcgaggtggccaccggcctcggcctggcgggcgtggcgtgggtcgacgacgacgtgacgttcagcgccggctgcatcggacggctgctggaggacttcctcgaccggggcgcgcggggctcggccggggcgagcaaggtgccccagcccaacccgtacgcggcggcccggttgctgcaccggggcaagcagatcaccaagacgccgaagttcggctacccgcacggctgctgcatggtgatcgacgccgaactcgccgcccgtggcatcccggaccgctacacctgcgaggacgactacttctgcttcgccctgctcgacccccggcacgacaacccgctgcaccgcacgcacatcgtcgcggacgccgtgagttaccacctgaccggcggaccggccggcgagatcagacaccgcgtcaaccgctcgctgctgaccgcggcggtgctgatggccgacttccccgccgacgtcgccggccggtactggcgcatgtacttctacgggctgtggccgctggcgcccgcagcccgcggccagggcctccacttcgcggccaagaagtggctgctcaaatcgatccatttcggctggttctcacgggtggcactggggctcgcagtacgcggcgtcgccggccggccccgacgtgagatcccgtggtcggcatacacccggcacgccaaaccaacggcctcgtcggtcccgttgtctcccaagatctcccgaggggaacgcgcatga

SEQ ID NO: 13 - DNA, 1776 bp, synthetic construct
  misc_feature 1..1776: note = P08-G05-PacBio_cluster016 - ctg4_253
  source 1..1776: mol_type = other DNA
gtgaccacgacgaccagaggcgcccgggcacgcctgctgttggccggccgtggccggcgttacgccgtgctcgccgccgtgtgggcggtgctgcggctgggcatgctggccctggggctgttgatgcagatggccttcgactccctcagcggcgagtcggccgccggcctgaacctgtggactctcgtcgcactcgtcgccggcatcgaggccgcgcgcatctccctgcagttcagcacggtggtgccgtggctcgaaccgcggatgcaataccacaccgtcggcgcgttgcggctgtcgctcttcgagtcgctgatcgaccccccgccggaggccggacacaccagcgcgggcgaggcggtcgcgaccgtcggcgccgacgccgcggacgccggcttcttcgtggtgtggtcgcccatcaacatcgcccgttggctgttcgcggtcgccgcgatcaccatcatgctgcgcatcgacgtgctcatcaccctgagcatcctcgggctgttcgtcgtgatcgtggcggtgatgcgcctggtgcaccggcggatgatggcctaccgcgagcagagccgcgcccagaccgcactggtcagcgaggcgctgcgcgaggtgatgggggtggcgctgggcgtgcaggccgcgcgcgccgaggacgcggtcggccggcgtctcgacgcgctcaacgccgttcgggccagggtcgccatctccgaggaactcgcggtcgcggtgcagcgcagcaccctgctgaacgccgccccgctcggcaccggcatcgtgctgctgatcgccgcaccggccatcagcagcaaccggctgtccatcggcgacatggtgctgttcaccttctacctgcagttgctcgtcgacacgctcgcctcgctcggcgtgctcaccgtccagttgcagcgggtgctcgtcgcgctgcgccggatgaccgcgatcacgggtggccgcctgcacccccggcacgggatggacctcgaccacgcccgacccggtgtcatgatcaacctttcgccgcgccgctcgcccccgccgcccgccggcggcccgccgacgatcacggtcaccgacctcaccgcccggcacgagggcgccgccaccggggtgtccggcatcggtttcacgatgccggccggccggctgaccgtcgtggtgggccaggtcggctcgggcaagaccacactgttgcgcgccctgctcggccaactgccgggtgccaccggcgaggtgcgctgggacgacgtcccgttgcccgcggaccgccgggcggtgctcgtgccgccccggtgcgcgtacacgccgcaggtgcctcgactgttcaccggcacgatccgggagaacatcctgctcgggctgccggacgaccggctggacgaggcggtgtcccggtcggccctcgacgccgacctgacggcgatggccgacgggctcgacacggtgatcgggcctcgtgggctacgcctgtccggtggccaggcgcaacgggtggccgtggcacggatgctcgtccgccgcaccccgctggtgctgtgcgacgacgtctccagcgcactcgatcccccaacgagcgaagccctgtgggacgggatcctgcgcgacggcgggagcacggtgctggcggtgtcccaccggcccgaggtgctgcgccgggccgccctcgtcgtggtcctgcgcgacggccggctggaggcggtcggggccctggacgacctgctggggagctccgacgagatacgccggctctggtccgccgtagccaccgactga

SEQ ID NO: 14 - DNA, 1740 bp, synthetic construct
  misc_feature 1..1740: note = P08-G05-PacBio_cluster016 - ctg4_254
  source 1..1740: mol_type = other DNA
atggcgtctcccgagccgcggacagccgcgacgaactgggcgcgctatgtgggctacgtccgcagtcaccggcgctggttcctgctgctggccgcgctgataccgctcgacgtcggcacccagttggtggcgccccaggtcgtacgccggttcatcgacgcggcggtgggcggccgctcgcagggcgagttggccgggtacgccatggtcttcgtggtgctcgcggtcgtgcggcaggtgctctcggtcggcaccggctgggtctccggccgggtcgcctggcgtacgaccaacggggtgcgccgcgacctgctcgcgcacgtcctgcggcaggggccgaagttcttcctccggcacccgcccggggaactggtcgaccggatggacggcgacgtcacccggttggccaagctcctgtccgagttggtgctcgacatcgccgtccaactgctgctgatcatcggcatcgtcgtggcgctgttcctgctcgactggcgctacggcgtgctcttcaccccgctcgcggtggtgatgctgctcctgctgcgacgcctgctcggccgcgcggtgccgctcatcgccgaccgccagcgggcggaggcgagcctgctcggctatctggaggagaacctgaccgggcgggaggacctcaacgccaacggcgggacggcgcacgccctggagggcgcctggcaacggatcctgcaccactaccggctggcccgccgcgccgcccgggccacggtcacctggccggcgacggtccaggcgctcaccgccgtcaacctctgtgcggcactggccaccggtgtgtggctcagccagcaccggggcgccagcgtcggcaccgtcttcgccggcctgtcgtacgcgatcctgctgcggatgcccctggtcagcatcacctcacgcttccaggacatggagaacgtcctgggcagcgtccgccgcatcgaggagctgttcggcgaggcgcacatcgtccccgacggcggtggcaccctcccgccgggccgcggcgcggtgagcttcaccggcgtcaccttcgcctacgacgaacgcccggtgctgtcggacgtgtcgttcacgatcccggcgggccggcgggtcgcgctggtcggacgcagcggcggcggcaagtcgaccatcctgcggatgctgttccgccagtacgacccgcaggccggctcggtctcgatcggcggcgtggacgtccggcagtgggacgtcgccgcgctgcgctcccgcatcacgctcgtcacccaggacgtgcacgtcatcgcggccaccctgcgggacaacctcaccttcttcgacgaccggtggcccgacgagcaactgcgccgggcggtcgaggaggtcggcctggacacctggtacgccgccctgcccgacggcctcgacacgcggatcgggccaggcggagcggggttgtccgccggtgaggagcagctgctcgcactggcccgggcactgctgaccgaccccgacatcgtgctgctcgacgagccgtcggcccggctcgacccgcacgcggagcggcggctgcgccgaacgatggagaggttcatcgagggtcgtaccgcgctgatcatcgcccaccggcccgagacgctctccatcgtcgacgacgtgatggtcgtcgaggacggccggatcgtccgcctggcgccccggtccgagctgacgggcggcagtgacgagacagccgcccggctgttccccgccgggcggtcctga

SEQ ID NO: 15 - DNA, 588 bp, synthetic construct
  misc_feature 1..588: note = P08-G05-PacBio_cluster016 - ctg4_255
  source 1..588: mol_type = other DNA
atggagctggtcgtgctactcgaccacgatggccaagccgtcggtgagcaggacaaacgcatcgttcacgacggcgacacgccgctgcatctcgcattctcctgttacgtcttcagcaatagcgggcagttcctaatgtcgcgccgttcgttgcggaaaaagacgtggccaggtatatggaccaacagcttctgcggccatcccggcccaggtgagccgatgaccgatgccgtccaccgacgcgcgtccgccgagctgggcctcacgctgagcgacctccgtctggtgctgccccgcttccgctaccgggccgaactcgacggtgtggtcgagaacgagatgtgccccgtctacgtggcccgggcagcggacgaaccagtgccgaacccggacgaggtggaccgggtcgagtgggtcgactgggactcgttccggaaccggacccggaccgacgggaccctctcgccgtggtgccggttgcaggtcgacgagctgaccgggatcggccccgtaccgggccggtggcccaccggcgacgccgccgagctgccacccgcagcccgggtcgcagtcgccgggcacggctga

SEQ ID NO: 16 - DNA, 1086 bp, synthetic construct
  misc_feature 1..1086: note = P08-G05-PacBio_cluster016 - ctg4_256
  source 1..1086: mol_type = other DNA
atgaccgccaccagcgagaccacccgaggcgtgcccggcctcgccgggcccgcgctcagagccgaggtggagcgcacgatcgacagcttcctgaccgcccggcgggacatgaacaccacccccgagtggcgcgacggggtcgacgccctccgcgccctggcactcgccggaggtaagcgcacccgaccgacgttctgctgcctgggttggcgcgcgttcggcggcggggaccaccccggcatcgtcgtggccgccacggcgctggaactgttccacctcttcgccctggtgcacgacgacatcatggacgacagcgatgtccggcgggggaacccgacgttgcacgtgacgatggccgaggcacaccgccggctcggctggcgtggcaacccggaacactacggccgtaacacggcgatcctctggggcgacctctgcctgacctggtccgacgaggctttccacggctgcggtctggcaccggagcgggtgtcggcggcggccgggttgctccaccgcatgcgcgtcgaggtgctgctcgggcagtacctcgacatccgcggcgaggctgtccgggcagacctggccgactgcctgacgatcctgctctacaagtcggcgaaatacaccgtggagcgcccgctgcagatcggcgcgatgctggccggcgcggcccgacccgacgtggaacgactctccggcttcggcgtcccgatcggtgaggcgttccagctgcgcgatgacgtcctcggtgtcttcggcgacgacgccgtcaccggcaagtcgatcctgaccgacctgcgcagcgggaagtccaccgtactcatggcgctgacccgggagcgggcgaacgccacccaggccgccgagatatcccggtggcacggcaacccgggcctcggcgagcgggaggcacaacggctgcggcacatcgtgcgggacaccggcgccctgcggacggtcgaggagatgatccacgcccgcaccgaagaggcggtggcggtgctcgactccctgcccatcgtcggggaggtgcgggtggcgctgaccgagctggcccacacgctgtcggtccggcgcagctga

SEQ ID NO: 17 - DNA, 948 bp, synthetic construct
  misc_feature 1..948: note = P08-G05-PacBio_cluster016 - ctg4_257
  source 1..948: mol_type = other DNA
atgacggttcacgacccccggcgcacggtgctggtgaccggtgccgccgggttcatcgggcgacacctggtcgagcacctgagcagccgggaccgcacggtgatcggtgtcgacctgcgccaggcgccgccccggccggcgaacgcggcagccggccggcacatcgtcgccgacctgcgcaccacggacatccgcccgctgctcgagggggtctcctcggtggtccatctcgccgcgctgcccggggtacggccgtcgtgggatcgcttcgacgactacgtcgaatccaacgtgctcgtcacccggcggctcctggaggcgggtgcgcagaccggtctacgacggctggtcatcgcctcctcgtccagcgtctacggcaaccagcgggacggggcgatgaacgaggaccaaccgcccgccccgatctccccgtacgcggtcaccaaactggccgcggaacacctcgcgctggcctacgcggcccgccctgcctccgtgctgaccaccatggccctgcggttcttcaccgtctacggcccccggcaacggcccgacatgctgatctcgcggatcatctcggcggtccaggacggccgggagatccgggtgttcggcgacggatcgcagcgtcgcgacttcgtcttcgtcaaggacgcggtgcgcgcggtgacggcggccatcgacgcgccgggcccgagccgcgtatgcaatgtcggcaccggtgagcagatatcggtcaccgggatcatcgcgctgatcagtgagctgatgggccgcccggcgaacgtccggaccgaatcccaccgggcaggtgacgtggtgtccacgcgggccgacaccgcccgcacggccgaggcgctcggcttccgcgcgtcggtctccctgcgggaagggctccgggagcacatcgacgcggtacgcagcgagtccatgcccgtcggcgcctga

SEQ ID NO: 18 - DNA, 798 bp, synthetic construct
  misc_feature 1..798: note = P08-G05-PacBio_cluster016 - ctg4_258
  source 1..798: mol_type = other DNA
atgggccggggacccgtgctgaccgtcatcgtgccgcacttccgctgcgccacctaccttgcggagtcggtcgcctccgtactcgggcagacccatcgggacctgcggctcgtggtcgccgacgatcactcgccggacgacgactggatcaccgcgctcgcaccgtggcgggacgatccgcggctgacgctgctgcgtgcgtcgcgcaacgtcgggccgctgcggctgaagaacgccgtggtcgagtcggtctccacgccgtacatcggcttccaggatgccgacgacgtgagcgaacccgaccgcttcgcccgccagcttgccgtactggagcggggccgcgccgatctggtcggctgcggctaccgcatcatcgatgcgcggggcaacccgcagggggtacggcacatgccgcgcaacggcaacctggtgcaccggttcggccgcaagacggtcgcgctgcatccggcgacggtcgtgcggcgcgaggtcttcacccgactcaacggcttcgacggcacggcggccttcggttccgacaccgacttccagctgcgggtcgcctggctctaccggattcgcaacctgcgcaggccgctctatcgctaccgcgcctgggcggcctcgctgaccgcagccccggagaccggcttcggatcacccgcccgggacgagtgcatgcggcggatggccgagttggagcagcgccgccggcaggcgcgcagccgcgcggatcggctcccgttgctggtggcaccgcccaacgacgtgtcgttcgcgctgcacgaggtggcgtga

SEQ ID NO: 19 - DNA, 699 bp, synthetic construct
  misc_feature 1..699: note = P08-G05-PacBio_cluster016 - ctg4_259
  source 1..699: mol_type = other DNA
atgaccaagcttttcaccgttgcggagatgatgggtcgggacctcgcccggcggcgggtagccctcgcgctgctcgcgttcgtaccactgctgttctacatcgcccggcacgacgccaatccggagaaggcgattcgcttcgccggcctgggtctggcctgggcggtgagcacggccgggttgttcctgagcaacaccacaaaggcggtggagcggcggctacgccaaatcggctatcggccggtgcacctctacctcggcacgctcaccgcgctgctgacgctgggtgccgtgctcgccgcggtgtactgggtgctcatctacgtcgaccaggacacgttgcagcggcccggcgccgtcgccatcgagttcgggctcacgatcgtggttgctgccccgctgggcctgctgatcagcgcggtggccccacacgatctggagggcacgctcgtcctgatctcgatcaccgggttgcagttcctggtcgatccggagcagaagctcggcaaggccctgccgttctggtcgactcaacaggtggggctctacggcctcgacggtggcgacccggaattcctgcgcaagggcctgctgcacggtgccctgtactgcgtcggcctcatgctggtcacggcggtgctggcgggtgtgcggctacggcggcgcaagcacatgcagatcagggcggccgtgcggtag

SEQ ID NO: 20 - DNA, 681 bp, synthetic construct
  misc_feature 1..681: note = P08-G05-PacBio_cluster016 - ctg4_260
  source 1..681: mol_type = other DNA
atggatctgagtgacaccgcttcggaagtgacggctaccaggcccggtcccgcggagggtgactcgtcgacaccgattcgactccatgcttcgaacattcacaagcggtacggcaagaatcacgtcctgaagggtgtcgagctcaccgtacgcgctggcgaggtggtcgccatcatcggcgccaacggctgcggcaagtcgacgctcatgaagatctgtgcgggtctggtcagcccgtccagcggtgtggtggaagtggacgagagcatggcctactgtccccagcaggcagggctcatgggcttcctgaccgccgacgaacacttcgcattgttcggtgccgggcacgggatgagccggggcgaggcccaggcgaccggccggcagctcgcgaagagcctggcctgggacggcggcatgtcgacgcaggcccggcacctgtccggtggcacccagcagaagctcaacctcattctcgcggcgctttccgaccccgacatcctcctcctcgacgagccctatcagggtttcgaccggggcagttacgtcgacttctgggactgggtgttacaacggcgcgacgccggcaagtcgaccctggtcatcacacacatgttgaattcactcgatcgggtcgacgccgttctcgacctcggagcgaaggcggccgccgactcatga

SEQ ID NO: 21 - DNA, 564 bp, synthetic construct
  misc_feature 1..564: note = P08-G05-PacBio_cluster016 - ctg4_261
  source 1..564: mol_type = other DNA
gtgaaccgcaatctcgtcacagcgttacaggctgcctggggactaccggagttccgcaacgggctgcgtcacatcatcgacctcgacgaggtggcggtgctcctggccacgctctccgtcccggaccacgaccacgaggtggaccgcagcctgctcagactgctgcggtccagcctggagatgggcgagatccgggaagcggtgttgctgctgctggagcaggacgaggtacgccgaccgctcgtcgccgcggtggtgccaccccgggccgaccgaccgggccaggctgccgccatcaccgcggcgtccgaggaccccgcagtgcggcgtgaggtacggacggtgctggacgacgcccgggtgcgcgcactgctctggcaggcgatcgaggcccaggtgaacgacgaccggggcggcctggtccgccgcgccgcggtgctgttcatacggcatccgagcgtgccccgcctcgcctgggccctgcaccgccacggcgtgctccgcgagctacgccgcccctccacgtcggccccggcggagggcaacggacagccgcgggcgtag

SEQ ID NO: 22 - DNA, 396 bp, synthetic construct
  misc_feature 1..396: note = P08-G05-PacBio_cluster016 - ctg4_262
  source 1..396: mol_type = other DNA
atgttcgagtaccacgggtggatcaccctccgctcgacagcggatgcggtcgacgacgacccaccccttcgcctcgacgagatccaggacctggtcaacagcctcgccgggtacgggctgatggaccttcgcccggccaacggcgcacaccacgtacacctcggcggtttccccaaccatcggggcacccggggtgccgacgttgtcgagctgttcaccaggatcggtcatctcgcccccgggtcgtacgggctactgtacgtccacgacgacgaggatcccgggcacgaactggtgttccgtgtccaccggctggtccgcggaaccgtctccgaacacaccgaccacctgctctcgccggtgatccccaccctcgaagacgagtacctcgactga

SEQ ID NO: 23 - DNA, 537 bp, synthetic construct
  misc_feature 1..537: note = P08-G05-PacBio_cluster016 - ctg4_263
  source 1..537: mol_type = other DNA
atgggaaaaccgacgctgttcctgaccgtgggactcccctgcaccgggaagaccacggctgctcggcgcatcgagatcgagcggaaagcccttcgtctgacgaaggacgagtgggtgaaggcgctctacggtcccgagaatccgccgttggcgggggaagtgatcgaggggcggctcatccagatcgggctgcgcgctctcgcgctcggcaccaatgtcgtgatcgactacggcctgtgggaccgcaacgagcgttccgccctgcgccaggccgcggtggacctcggcgcgagggcggagttgcgctacttcgcactgaccccttccgcgcagcggagacgacgtgaccagcgccaggccgaggcgccacacaccacctggcccatgtccgacgcggaactcgtcgagtgggcaacccgcatggacatccccacgccgggtgaactcgacggcagcgagcccatcgacggcccacctgcgggcttcacgacgtgggacgagtggcgcagccatcgttggccgccgtcggtccactga

SEQ ID NO: 24 - DNA, 579 bp, synthetic construct
  misc_feature 1..579: note = P08-G05-PacBio_cluster016 - ctg4_264
  source 1..579: mol_type = other DNA
atgatttccggacggagtttcgcggggtggtaccgccaccgcctggtgtgggtggcggtgatcctgctggcggtgtcggccggcacggtcgtactggccaaccgggccgatccgaaggcggagccggcggatctgcaggcgcagatcgtggcccggatgcgcaccacgctcgaacaggcggacccggggcagcaccagcacgcgggccacggcaccccgcagggtgccggcacggagaagccaccggtgatctgcggggtccgcgtctacggcttcgagccggcggaggccaccgcgttcgccgacgtccggacgatctacggctttcacctctgcggaatcgccgagcagaagcgcccctgggacgtggcggtcaagctggccggcccattgatcatggacatgtccaccgatcccccgggcatccaggtcgtcgaggcgactgccgaggtcaggttcgtcgaccggctgcgggagatgttcccgcccgcgtacgcgacgatggcgcagcaggaggctttgggcgcctcggagatggccgacctgcgtcgccggtacgacaccgcagccaagtcatga

SEQ ID NO: 25 - DNA, 681 bp, synthetic construct
  misc_feature 1..681: note = P08-G05-PacBio_cluster016 - ctg4_265
  source 1..681: mol_type = other DNA
gtgcgttcacacaggtccgaccaatccggcggacggccccggtccaccgcccgccgatccctccaggcgctcgtcctgatgttcgccatggtggtcggtacgctgcccgtgaccagcgcagcccaggcgcacggcaccatcatcaatccggcgacccgcgcctaccagtgctggaatacctggggcagccaacacaccaaccccgccatgcagcagcaggacccgatgtgctggcaggcgttccaggccaacccggacaccatgtggaactggatgagccaactgcgcgacggcctcgccggccagtaccagcccagcaccccggacgggcagctgtgcagcaacgccctgtcccggaacaactcgctgaaccagcccggggcgtggaaggcgaccaccgtcagccgcaacctcacggtccagatgtacgaccaggccagccacggtgctgacttcttccgggtctacgtcaccaagcaggggttcaaccccgccacccagaaggtcgggtggggcaacctcgacctgatcaccacgaccggacggtacgcgccggcacagaacatctcgttcaacgtctcgatcccgtcctcgcgcagcggaagccacgtgctgttcttcatctggaaggcgtcccacgccgaccagacctacatgtggtgcagcgacatcaagatctga

SEQ ID NO: 26 - DNA, 501 bp, synthetic construct
  misc_feature 1..501: note = P08-G05-PacBio_cluster016 - ctg4_266
  source 1..501: mol_type = other DNA
atgagactgaagagcagccttctcggtgtcgctgcggcggccgtgacgctggccgccgccatcgacggcgccagcgcgtcgaccacggcgccggccggcatgaccccatcccccagcaccgctccgccgccgagcaccgccctgcccagcagcccgccaccgagcagcccgccaccgaccggggccgcgtgcaccgtccggtactcggtctacacctgggacaccggtctgaccagcagtatcaccatcaccaacaccggcgtcaccacgatcacctcgtggaggctggcgttcccgctgcccgccggacaacgcatcaccaccggctggaacgcggtgtactcgccatccagtggcaacgtcacggcgcgtggcctggacgaccacgtcatcatcgggccgggcgcgtcgatcgccatcgggttcgtggccgaacacaccggaggcaccggcagtccgtcctcgttcaccctgaacgggacatcctgctcgctgagctga

SEQ ID NO: 27 - DNA, 1029 bp, synthetic construct
  misc_feature 1..1029: note = P08-G05-PacBio_cluster016 - ctg4_267
  source 1..1029: mol_type = other DNA
atgggcaaggtaggaatgcgcgtccgccgctcgacgctggccgtactcacgggcgcggcgctggccgggctgctggcggtcaccgcagccgccgcggcacccggcgaggcaccgcagggcaaggccgccgcggcggcccgtaccgacgtctatctcaaggacgtcgccgccgacaccggcgtccaaccacactcgtacggcccgctgtgggccagccccgacatcaaggtctgcccgaccgccgtcgaatgtgccgtcagtcagaacccgatcgtcggcgtcaccaactacctcttcctgaaggtgcgcaacgccggcccgtacggctcgggcatcgacaacggagtgctgcgggtctaccggaccaccccgggcggcggcgccacctggccgacgcactggacccagatcggcgcggccagcgtgcccgtctacccgggcgtcaccacggtcacgatcccgtggaccggggtgcccggaccgggccacttctgcctgttggcacgctgggtgtcccccaccgacccgatgaacttcgagggcccgagcaccagcgtcaacacccagtacaacaacaacatcgcctggcgtaacgtcgactcggtccggctcacgccgggcgggccacgggtcgtccggccgttcgccatcggcaacgtgaccgccgcggagaccaagaacgacctggtcttcacccagccggcagagcaggcccggggtgcccgggtcttcgtcgacctcggcccgaccctgttcgcgcgctggcagtccaccggcatggtcggtacgggaatccgccaggtcggcaagaccgagttggagatcgtggaaccgacgaaggccagcctcagcgggctggtgttgaagcccggtgagcgcctcgagttctccctgtccttctcggccgacaccgccctggccaaggagaccgcgttcaacgtgacccagttcgggccggacgccgacggcactgcgcgcgccgacctcggtggggtgcagtacctgctggcgggcgagcagcgggggtaa

SEQ ID NO: 28 - DNA, 465 bp, synthetic construct
  misc_feature 1..465: note = P08-G05-PacBio_cluster016 - ctg4_268
  source 1..465: mol_type = other DNA
atggagccgtggccgccggtcgatgacgatgcactgaccacggagctgggcgcggcgttgtgggacaccgggccggtctcggaggcgtacctgagcgcggcgttggcagcgttcacctggcgtaccgcggacgcggaactggccgtcgccgagctgacgttcgactcggccatcgacgccgagttggtggggatgacccggtccgccgcgcccgtgcggacactgtccttccgcagcgggtcggtggtggtggagatcgaggtcagcgacaccgggatcgtcgggcagctctccccgtccggtggtggacgggtgagtgcccgcacggcctccggcacctacgacgaggcaccggtggacgcggtgggattcttctcgttgggcgtcccgccgccggggctggtccagttgtgcgcgcgtaccgtcgggtttgcggtgtccaccgcctgggtctgtctgcgctga

SEQ ID NO: 29 - DNA, 642 bp, synthetic construct
  misc_feature 1..642: note = P08-G05-PacBio_cluster016 - ctg4_269
  source 1..642: mol_type = other DNA
atgaccggtgccagcgcgaccaccctgctgtccgccgccgccggcggggacgagtcggcctgggccgaactggtgcaccggtacaccccgatcgtgtactcggtcatccgggcgtacgacctcagcagggccgatgcggccgatgtcaaccagaccgtctggctccgcctggtcgagcagttgggccgggtacgcgagcctgaggcgctgccgggctggctggccaccacgacccggcgcgagtgctaccggttgtcccgcctcagccgccgggcccaacccgtcgacccgtacgaccagtcgcttgagccgtaccacgggttctcccgggagttggccgcgatggctccggccccggacgacgagttgctgcgcgcggagcggcgacaggcgttgcgcgagggtttcgtgcagctcccgccacgctgccaggcgttgttggcgctgctgacggccgacccgccgatgagctaccgggaggtcgccgagcggttgggcgtgccggtcggcagcatcggccccacccaggcccgttgcctgcgtaggctgcgggcatgtccggcgatcgtcgagtttctcggagcgtccaccatcggtacgaacgggggggaaccggatggagccgtggccgccggtcgatga

SEQ ID NO: 30 - protein, 564 aa, synthetic construct
  REGION 1..564: note = P08-G05-PacBio_cluster016 - ctg4_242
VSKISPGTAILRRAVARNGRRLWGGTALAGVYQLCAALVPILIGVIVDRAVATGDLTALLTWITTLAMVYLVLAVTYRLGARQLQRAIAEEGHRLRVELAARILDPRVLRTERHTGDLLSVTTTDAEYTSYLLDHIPRITSALVAVTVSAVALLLISPPLGLTVLVAIPIVLAILNLTAPLIARRVKEQQDQAGRATALATDLVTGLRPLRGIGAQDAGAARYRRVSRRSLAAALRASRTQNAYLSASTMSSTLLAGGIAVMAGWFALTGRITVGQFITVIGSAQFLVEPFGVLAVVPSWTAAARASADRVAAVLQAGVVLPPGVAAPPDTGYELRLTDVTYGPLAGLHLHVRVGEFVGVVARRPADAEALARLLAVPDAYQGDILLGGERLETVERDRARRVLHSEPHRTDLFTGTLASNVAIHDDDGGRLAAALRAAAADEVAHLHPAGLHMPVTERGANLSGGQRQRVALARALLARPPLLVLHDPTTAVDAVTEQAIADGIRALRHGPDASYGTVLITSSPTLLAATDRVVFLGDGTVTAEGAHAELVTRDDSYRRAVRR

SEQ ID NO: 31 - protein, 619 aa, synthetic construct
  REGION 1..619: note = P08-G05-PacBio_cluster016 - ctg4_243
MTHPTLPVASPRESRAWLGAQLRARAVATGATLVVGLLAAATAIVPAYALGLLVDRVRAGAGTDALVPVAVVIGAAAVVGGLVAGVSAHLIRGLGARILADLRERALDAALRLPVAVLDRVGRGDLVSRVGADISAIDRAVSEVLPTMISALLLAAISLGTIIGIDWRLGLAGAVAVPLYVLGLRWYLPRAAPAYAAERVAIAQRSQQFVESVQGLRTVHAYRWEDRHLAGIAAASARARDISTSVFALYTRFVGRINGAELAGLGAVLVVGFLLVRRDAVTLGQVSAAALLLHRLFGPVLQLMVTFDKAQDAGASLARLVGVLRTDPERRAGGTTPPAHDLVLTDVGFSYDGTTPVLRDISLRVGPGQLVALVGATGAGKTTVASIAAGILRPEHGAATLGGVPVTELPARTIALVSQETHVFAGPLGEDLLLARPHASPEELTAALARVGALSWALALPDGLDTVVGEGGHPLTAAQAQQLALARVVLLDPAVVVLDEATAEAGSAGARDLEQAAAAALEGRTALVVAHRLTQAVTADHIIVMEQGRIVESGPHADLVGAGGNYARLWAAWADRATADPGPTRTGAAAGRAVDSGGPSTPQPRHGRWTGPPLTAQRE

SEQ ID NO: 32 - protein, 202 aa, synthetic construct
  REGION 1..202: note = P08-G05-PacBio_cluster016 - ctg4_244
VNHPRPKARLSRALGIPLTRKCVRYFERRPYPPGVHGRNRRKTSDYQVRLLEKQRLRHQYNVSEAQLRRTFDEAARGAGKTGESLVALLERRLDALVLRAGLARSIYQARQLVGHGHFTVDGRKVDRPSYRLRPGQVVAVRERSRALPPFQLAAAGAHADDRPRPYLSVELTGLSATLLREPARHEVPVICDEQLVVEFYSR

SEQ ID NO: 33 - protein, 244 aa, synthetic construct
  REGION 1..244: note = P08-G05-PacBio_cluster016 - ctg4_245
MRPSPAEIVRTLVAGRLPALAHLAGSTGPHQVRHATDPDGRVLLLVPALSDLAAALAPPAGDTDVATALDVLDLPPTAGAPSLGRAWVSGWAERLDGDEARQAAMDFATTQPTGDLLDVGSRFQLHRFEVAEARWEHTGGIRPIDPQAYAQAEPDPLHAAEAGLLADLADHHAEQVAGHLRRQLKLSGAESPRVVRIDRYGLLVAYGRPGATRRARVSFAGPVADVAELSRLLHPMLCPRQAAA

SEQ ID NO: 34 - protein, 433 aa, synthetic construct
  REGION 1..433: note = P08-G05-PacBio_cluster016 - ctg4_246
MTPHQPLARLLAGKLVDPAPEGWQRDFGRLRCQEPQAVVFAARVADVQETVRYALVGGLPVVARGNGCSSNGQSLTDGLQLDTAALDRIELVGAATVRVEAGVRWGTLHRWLHEHGLALPVVTSNEHASVGGTLSVGGFGPTSPFVGAVVDQVVALEVVTGDGALVHATADGPHRELFSYALCGLGQVGLIVRAELRVVPAEPAVLVRRLDGDDLATVAEQVWADERLRLCRAVYSTAARRWKVEVGRSARDAAAESDETLVADYHQRLFEAEAGFSVGIARGQVAAGLLTDETEARRLWSDFFLPMEKADAFFAGVRDRFADPAVVPVVNGAFLRAGDRPRPPLSPLPDADRVRTFGFYCIVPPDRVDEYREHFDAAGALCHELGGRQYLHGYHRVERDFYRAQFGGPTVERWREVKRRYDPGAVLGPQLPI

SEQ ID NO: 35 - protein, 269 aa, synthetic construct
  REGION 1..269: note = P08-G05-PacBio_cluster016 - ctg4_247
VLTFPEFKAEMDDSQARVVQRCLAELTSLGMSRQGLDFVRDLPGYPRTPYYTTLWVRDPRVRADLGAGVSIHAVGVKLIDDLIDDDQPLSPRDQIFGVQLLSVGTTLLASQQRPVEVLRTLEDDYRDIWRQEIVEVSAPRPTTVESWIAAARIKAGVMLANYAAVNCLAGGVPEMVTAAREFGEALGVLYMIGDDAVDFDEIDERDGNLAHLVASGAADLADVAAAVDEWRDRAVAAVRQGDPVCDLVPFFDHFAAKLKGLISRPTATV

SEQ ID NO: 36 - protein, 271 aa, synthetic construct
  REGION 1..271: note = P08-G05-PacBio_cluster016 - ctg4_248
VLSGVSGLDDEWAVAQRVLLERVTDRLAGIGMTPDGVEYVRQMKGYPRIPYFASLWVPDPVQRQELGIAICTHAMGIKLVDDLMDGDQPIRPWDQILGMFLTQSSTATFGAHHRPAEVLRVFDEDYEVIWREQINEAERPAHDLDSWIHYARIKSGRMMMCYASAACLAADVVERLPAARAFCEAFGVLFMIGDDIRDHRDLGEAGGNLCHLIRSGQVSRDAAVAATRRWRAEALRALAEFPPEFPIAGFMADFADRLITFLLEDDRVDVS

SEQ ID NO: 37 - protein, 421 aa, synthetic construct
  REGION 1..421: note = P08-G05-PacBio_cluster016 - ctg4_249
MTRSVQVISMRTWGELGNLLAGRTLAATLAAGLDDAEVTVVEAETLFPQFAEAGAAIRELVAARLPADEVWAGYLRLMTDFTDRLPAGLDDDPLPAATVDRLAPVADHFAQTKPDLVIGTKGVISRLSHAALRIADLPTPVVNYVTNEGLLRLPVHRAPHLPVHLVQFDRAKDYLVREHGYRPERVVPVGRLLAQAQAAQVFEGAAAGIGDADAVGAADPALTAAGTRILILSNRGGREYLAALERIARAHPEAALVFIAYNNPALAAEAAELLTALGRSAWQTFDRLAQGDYLRYLAWLHAADAPLFISKTGPNSMLEGLYFGVPQLLVRSGLPMEEWVGPFLAEHGVGRGFDRMEELVEVAVRWVGDPDELPRRRRRAMALARTLLDPDRARRQTVAAVAAVLDGVPLRIDEEETCSPE

SEQ ID NO: 38 - protein, 709 aa, synthetic construct
  REGION 1..709: note = P08-G05-PacBio_cluster016 - ctg4_250
MREVRLPGNLSTVPATAPVLTALRAQAPGLLHVVDPYKVPLAEAVEKSRAVADLGHPALLVGSTDNAAFDRQVVPYVAELRERTDLPLVLHFPSIPGRGAPLCPGAAAVLVHAVPNSVRSFFVSGGLTATVRAATALGQDGPELLRSASFAFGSDGKTHEAVGAPPTVQDGAELPRLVAAVAAADFDLAYLLYRHGRVPEAVLRAYRRGLAPQQLLFVGGGVTSYRQVREYHDCGVDWVVVGNGLETADWRETLRRLSGRPGDRAGDPAAPPTLAAELQRGLRRPVRTDLPGRQAYATNVGRLHELLPAVVAPVTSAEEVAHVLRTTRAAGTPVAVRGSGHSFGLQGLSDGGVLIVGEPGDPGVRLRDEGTAEVSARAPWYAAEQALNAQGRSIPVLTNHLTVSIGGTLSVGGYGEGTIRYGGQVDLVERFRLILPDGQARWCSPVESPELFRYGIGSLGQLGVLDRVVLRTVDYQRHTTIATRRHPSLTALVGSIGWLAEEQRSPVDLFSAQHYEGGFIATYGMRGDGPLPPQLRRQTETRVEADWPLRSMRESRRNPADPSVHFIWADYCLSLPAAAEFARFLDAEILPSPGYREGAGRLLMLGVHRPEQRPGVPFTPAAGLRGLAVGFGLYFSVPRGDGAGVARVQGLHRQVLAECLALGGRPYLAGWYDLDEADLQSMYGADLAAVRRLRRELDPHGLFNPGLLA

SEQ ID NO: 39 - protein, 278 aa, synthetic construct
  REGION 1..278: note = P08-G05-PacBio_cluster016 - ctg4_251
MTDIAKYIKQRAQGKGVLNVLSGVSRTYFHGTGTKALSDAAWLKQKSSRTPPQFSMIAIETMSGCNYRCSFCPIGTLKMPSGQMSMELYESLLDQLADFTGVIHPYLFNEPLLDRRLPAMVRMAKERTKASISIQTNGSKLTEELADELTRYATVVVNDYTTDLSVLEKVRRYPARPELVLVDRDPDATLTNRAGNVLGRPTVLLNQFCVRPFNEFYITQDGRVVLCCQDWSVQEVMGNVNDNTIEEIWNNEKYTARRASLLRKERTDLCAKCDFPGI

SEQ ID NO: 40 - protein, 398 aa, synthetic construct
  REGION 1..398: note = P08-G05-PacBio_cluster016 - ctg4_252
MLELLTRPVRWAIRRGYQPADGDPPPTECIVVRPGPAGVRAATAVPAVAAELGDGFGGGLALVLPTYVGRAPLSSHRETVEELVTQLTDLPPTVAARPLVLVVGMQWRDDQHDEAIARLGQLSEIAASRGGLTFVGIAMPQAGKSRMLNAAFEVATGLGLAGVAWVDDDVTFSAGCIGRLLEDFLDRGARGSAGASKVPQPNPYAAARLLHRGKQITKTPKFGYPHGCCMVIDAELAARGIPDRYTCEDDYFCFALLDPRHDNPLHRTHIVADAVSYHLTGGPAGEIRHRVNRSLLTAAVLMADFPADVAGRYWRMYFYGLWPLAPAARGQGLHFAAKKWLLKSIHFGWFSRVALGLAVRGVAGRPRREIPWSAYTRHAKPTASSVPLSPKISRGERA

SEQ ID NO: 41 - protein, 591 aa, synthetic construct
  REGION 1..591: note = P08-G05-PacBio_cluster016 - ctg4_253
VTTTTRGARARLLLAGRGRRYAVLAAVWAVLRLGMLALGLLMQMAFDSLSGESAAGLNLWTLVALVAGIEAARISLQFSTVVPWLEPRMQYHTVGALRLSLFESLIDPPPEAGHTSAGEAVATVGADAADAGFFVVWSPINIARWLFAVAAITIMLRIDVLITLSILGLFVVIVAVMRLVHRRMMAYREQSRAQTALVSEALREVMGVALGVQAARAEDAVGRRLDALNAVRARVAISEELAVAVQRSTLLNAAPLGTGIVLLIAAPAISSNRLSIGDMVLFTFYLQLLVDTLASLGVLTVQLQRVLVALRRMTAITGGRLHPRHGMDLDHARPGVMINLSPRRSPPPPAGGPPTITVTDLTARHEGAATGVSGIGFTMPAGRLTVVVGQVGSGKTTLLRALLGQLPGATGEVRWDDVPLPADRRAVLVPPRCAYTPQVPRLFTGTIRENILLGLPDDRLDEAVSRSALDADLTAMADGLDTVIGPRGLRLSGGQAQRVAVARMLVRRTPLVLCDDVSSALDPPTSEALWDGILRDGGSTVLAVSHRPEVLRRAALVVVLRDGRLEAVGALDDLLGSSDEIRRLWSAVATD

SEQ ID NO: 42 - protein, 579 aa, synthetic construct
  REGION 1..579: note = P08-G05-PacBio_cluster016 - ctg4_254
MASPEPRTAATNWARYVGYVRSHRRWFLLLAALIPLDVGTQLVAPQVVRRFIDAAVGGRSQGELAGYAMVFVVLAVVRQVLSVGTGWVSGRVAWRTTNGVRRDLLAHVLRQGPKFFLRHPPGELVDRMDGDVTRLAKLLSELVLDIAVQLLLIIGIVVALFLLDWRYGVLFTPLAVVMLLLLRRLLGRAVPLIADRQRAEASLLGYLEENLTGREDLNANGGTAHALEGAWQRILHHYRLARRAARATVTWPATVQALTAVNLCAALATGVWLSQHRGASVGTVFAGLSYAILLRMPLVSITSRFQDMENVLGSVRRIEELFGEAHIVPDGGGTLPPGRGAVSFTGVTFAYDERPVLSDVSFTIPAGRRVALVGRSGGGKSTILRMLFRQYDPQAGSVSIGGVDVRQWDVAALRSRITLVTQDVHVIAATLRDNLTFFDDRWPDEQLRRAVEEVGLDTWYAALPDGLDTRIGPGGAGLSAGEEQLLALARALLTDPDIVLLDEPSARLDPHAERRLRRTMERFIEGRTALIIAHRPETLSIVDDVMVVEDGRIVRLAPRSELTGGSDETAARLFPAGRS

SEQ ID NO: 43 - protein, 195 aa, synthetic construct
  REGION 1..195: note = P08-G05-PacBio_cluster016 - ctg4_255
MELVVLLDHDGQAVGEQDKRIVHDGDTPLHLAFSCYVFSNSGQFLMSRRSLRKKTWPGIWTNSFCGHPGPGEPMTDAVHRRASAELGLTLSDLRLVLPRFRYRAELDGVVENEMCPVYVARAADEPVPNPDEVDRVEWVDWDSFRNRTRTDGTLSPWCRLQVDELTGIGPVPGRWPTGDAAELPPAARVAVAGHG

SEQ ID NO: 44 - protein, 361 aa, synthetic construct
  REGION 1..361: note = P08-G05-PacBio_cluster016 - ctg4_256
MTATSETTRGVPGLAGPALRAEVERTIDSFLTARRDMNTTPEWRDGVDALRALALAGGKRTRPTFCCLGWRAFGGGDHPGIVVAATALELFHLFALVHDDIMDDSDVRRGNPTLHVTMAEAHRRLGWRGNPEHYGRNTAILWGDLCLTWSDEAFHGCGLAPERVSAAAGLLHRMRVEVLLGQYLDIRGEAVRADLADCLTILLYKSAKYTVERPLQIGAMLAGAARPDVERLSGFGVPIGEAFQLRDDVLGVFGDDAVTGKSILTDLRSGKSTVLMALTRERANATQAAEISRWHGNPGLGEREAQRLRHIVRDTGALRTVEEMIHARTEEAVAVLDSLPIVGEVRVALTELAHTLSVRRS

SEQ ID NO: 45 - protein, 315 aa, synthetic construct
  REGION 1..315: note = P08-G05-PacBio_cluster016 - ctg4_257
MTVHDPRRTVLVTGAAGFIGRHLVEHLSSRDRTVIGVDLRQAPPRPANAAAGRHIVADLRTTDIRPLLEGVSSVVHLAALPGVRPSWDRFDDYVESNVLVTRRLLEAGAQTGLRRLVIASSSSVYGNQRDGAMNEDQPPAPISPYAVTKLAAEHLALAYAARPASVLTTMALRFFTVYGPRQRPDMLISRIISAVQDGREIRVFGDGSQRRDFVFVKDAVRAVTAAIDAPGPSRVCNVGTGEQISVTGIIALISELMGRPANVRTESHRAGDVVSTRADTARTAEALGFRASVSLREGLREHIDAVRSESMPVGA

SEQ ID NO: 46 - protein, 265 aa, synthetic construct
  REGION 1..265: note = P08-G05-PacBio_cluster016 - ctg4_258
MGRGPVLTVIVPHFRCATYLAESVASVLGQTHRDLRLVVADDHSPDDDWITALAPWRDDPRLTLLRASRNVGPLRLKNAVVESVSTPYIGFQDADDVSEPDRFARQLAVLERGRADLVGCGYRIIDARGNPQGVRHMPRNGNLVHRFGRKTVALHPATVVRREVFTRLNGFDGTAAFGSDTDFQLRVAWLYRIRNLRRPLYRYRAWAASLTAAPETGFGSPARDECMRRMAELEQRRRQARSRADRLPLLVAPPNDVSFALHEVA

SEQ ID NO: 47 - protein, 232 aa, synthetic construct
  REGION 1..232: note = P08-G05-PacBio_cluster016 - ctg4_259
MTKLFTVAEMMGRDLARRRVALALLAFVPLLFYIARHDANPEKAIRFAGLGLAWAVSTAGLFLSNTTKAVERRLRQIGYRPVHLYLGTLTALLTLGAVLAAVYWVLIYVDQDTLQRPGAVAIEFGLTIVVAAPLGLLISAVAPHDLEGTLVLISITGLQFLVDPEQKLGKALPFWSTQQVGLYGLDGGDPEFLRKGLLHGALYCVGLMLVTAVLAGVRLRRRKHMQIRAAVR

SEQ ID NO: 48 - protein, 226 aa, synthetic construct
  REGION 1..226: note = P08-G05-PacBio_cluster016 - ctg4_260
MDLSDTASEVTATRPGPAEGDSSTPIRLHASNIHKRYGKNHVLKGVELTVRAGEVVAIIGANGCGKSTLMKICAGLVSPSSGVVEVDESMAYCPQQAGLMGFLTADEHFALFGAGHGMSRGEAQATGRQLAKSLAWDGGMSTQARHLSGGTQQKLNLILAALSDPDILLLDEPYQGFDRGSYVDFWDWVLQRRDAGKSTLVITHMLNSLDRVDAVLDLGAKAAADS

SEQ ID NO: 49 - protein, 187 aa, synthetic construct
  REGION 1..187: note = P08-G05-PacBio_cluster016 - ctg4_261
VNRNLVTALQAAWGLPEFRNGLRHIIDLDEVAVLLATLSVPDHDHEVDRSLLRLLRSSLEMGEIREAVLLLLEQDEVRRPLVAAVVPPRADRPGQAAAITAASEDPAVRREVRTVLDDARVRALLWQAIEAQVNDDRGGLVRRAAVLFIRHPSVPRLAWALHRHGVLRELRRPSTSAPAEGNGQPRA

SEQ ID NO: 50 - protein, 131 aa, synthetic construct
  REGION 1..131: note = P08-G05-PacBio_cluster016 - ctg4_262
MFEYHGWITLRSTADAVDDDPPLRLDEIQDLVNSLAGYGLMDLRPANGAHHVHLGGFPNHRGTRGADVVELFTRIGHLAPGSYGLLYVHDDEDPGHELVFRVHRLVRGTVSEHTDHLLSPVIPTLEDEYLD

SEQ ID NO: 51 - protein, 178 aa, synthetic construct
  REGION 1..178: note = P08-G05-PacBio_cluster016 - ctg4_263
MGKPTLFLTVGLPCTGKTTAARRIEIERKALRLTKDEWVKALYGPENPPLAGEVIEGRLIQIGLRALALGTNVVIDYGLWDRNERSALRQAAVDLGARAELRYFALTPSAQRRRRDQRQAEAPHTTWPMSDAELVEWATRMDIPTPGELDGSEPIDGPPAGFTTWDEWRSHRWPPSVH

SEQ ID NO: 52 - protein, 192 aa, synthetic construct
  REGION 1..192: note = P08-G05-PacBio_cluster016 - ctg4_264
MISGRSFAGWYRHRLVWVAVILLAVSAGTVVLANRADPKAEPADLQAQIVARMRTTLEQADPGQHQHAGHGTPQGAGTEKPPVICGVRVYGFEPAEATAFADVRTIYGFHLCGIAEQKRPWDVAVKLAGPLIMDMSTDPPGIQVVEATAEVRFVDRLREMFPPAYATMAQQEALGASEMADLRRRYDTAAKS

SEQ ID NO: 53 - protein, 226 aa, synthetic construct
  REGION 1..226: note = P08-G05-PacBio_cluster016 - ctg4_265
VRSHRSDQSGGRPRSTARRSLQALVLMFAMVVGTLPVTSAAQAHGTIINPATRAYQCWNTWGSQHTNPAMQQQDPMCWQAFQANPDTMWNWMSQLRDGLAGQYQPSTPDGQLCSNALSRNNSLNQPGAWKATTVSRNLTVQMYDQASHGADFFRVYVTKQGFNPATQKVGWGNLDLITTTGRYAPAQNISFNVSIPSSRSGSHVLFFIWKASHADQTYMWCSDIKI

SEQ ID NO: 54 - protein, 166 aa, synthetic construct
  REGION 1..166: note = P08-G05-PacBio_cluster016 - ctg4_266
MRLKSSLLGVAAAAVTLAAAIDGASASTTAPAGMTPSPSTAPPPSTALPSSPPPSSPPPTGAACTVRYSVYTWDTGLTSSITITNTGVTTITSWRLAFPLPAGQRITTGWNAVYSPSSGNVTARGLDDHVIIGPGASIAIGFVAEHTGGTGSPSSFTLNGTSCSLS

SEQ ID NO: 55 - protein, 342 aa, synthetic construct
  REGION 1..342: note = P08-G05-PacBio_cluster016 - ctg4_267
MGKVGMRVRRSTLAVLTGAALAGLLAVTAAAAAPGEAPQGKAAAAARTDVYLKDVAADTGVQPHSYGPLWASPDIKVCPTAVECAVSQNPIVGVTNYLFLKVRNAGPYGSGIDNGVLRVYRTTPGGGATWPTHWTQIGAASVPVYPGVTTVTIPWTGVPGPGHFCLLARWVSPTDPMNFEGPSTSVNTQYNNNIAWRNVDSVRLTPGGPRVVRPFAIGNVTAAETKNDLVFTQPAEQARGARVFVDLGPTLFARWQSTGMVGTGIRQVGKTELEIVEPTKASLSGLVLKPGERLEFSLSFSADTALAKETAFNVTQFGPDADGTARADLGGVQYLLAGEQRG

SEQ ID NO: 56 - protein, 154 aa, synthetic construct
  REGION 1..154: note = P08-G05-PacBio_cluster016 - ctg4_268
MEPWPPVDDDALTTELGAALWDTGPVSEAYLSAALAAFTWRTADAELAVAELTFDSAIDAELVGMTRSAAPVRTLSFRSGSVVVEIEVSDTGIVGQLSPSGGGRVSARTASGTYDEAPVDAVGFFSLGVPPPGLVQLCARTVGFAVSTAWVCLR

SEQ ID NO: 57 - protein, 213 aa, synthetic construct
  REGION 1..213: note = P08-G05-PacBio_cluster016 - ctg4_269
MTGASATTLLSAAAGGDESAWAELVHRYTPIVYSVIRAYDLSRADAADVNQTVWLRLVEQLGRVREPEALPGWLATTTRRECYRLSRLSRRAQPVDPYDQSLEPYHGFSRELAAMAPAPDDELLRAERRQALREGFVQLPPRCQALLALLTADPPMSYREVAERLGVPVGSIGPTQARCLRRLRACPAIVEFLGASTIGTNGGEPDGAVAAGR

(19) United States
(12) Patent Application Publication
(10) Pub. No.: US 2026/0109688 A1
(43) Pub. Date: Apr. 23, 2026

(54) NOVEL ANTIBIOTIC COMPOUND

(71) Applicant: SINTEF TTO AS, Trondheim (NO)

(72) Inventors: Alexander WENTZEL, Heimdal (NO); Kristin Fløgstad DEGNES, Trondheim (NO); Giang-Son NGUYEN, Trondheim (NO); Geir KLINKENBERG, Trondheim (NO); Håvard SLETTA, Trondheim (NO); Trond Erling ELLINGSEN, Ranheim (NO); Mario Dino VAN DISSEL, Leiderdorp (NL); Gilles VAN WEZEL, Heemstede (NL); Marnix MEDEMA, Bennekom (NL); Antonio FERNANDEZ-GUERRA, Copenhagen (DK)

(21) Appl. No.: 18/864,850

(22) PCT Filed: May 16, 2023
(86) PCT No.: PCT/EP2023/063186

§ 371 Date: Nov. 11, 2024

(30) Foreign Application Priority Data
May 17, 2022 (GB) 2207185.6

(51) Int. Cl. C07D 405/14; A61K 31/7036; A61P 31/04; C12N 1/20; C12N 15/76; C12R 1/465

(52) U.S. Cl. CPC C07D 405/14; A61K 31/7036; A61P 31/04; C12N 1/20; C12N 15/76; C12R 2001/465

(57) ABSTRACT

The present invention provides a new antibiotic compound termed nidaromycin derived from a new biosynthetic gene cluster (BGC), and its uses. Also provided herein are novel genes and nucleic acid molecules encoding the biosynthetic machinery for the production of nidaromycin, and to constructs, vectors, and host cells for expressing the BGC and methods for producing the compound.

### CROSS-REFERENCE TO RELATED APPLICATION

The present application is a § 371 national phase of International Application No. PCT/EP2023/063186, filed on May 16, 2023, which claims the benefits of United Kingdom Application No. 2207185.6, filed on May 17, 2022, which applications are incorporated by reference herein.

### INCORPORATION BY REFERENCE OF SEQUENCE LISTING PROVIDED AS A SEQUENCE LISTING XML FILE

A Sequence Listing is provided herewith as a Sequence Listing XML, “155509-01 ST.26 Sequence Listing.xml”, created on May 4, 2023, and having a size of 143,206 bytes. The contents of the Sequence Listing XML are incorporated herein by reference in their entirety.

### FIELD

The present disclosure and invention relates to a new antibiotic compound, which we have termed nidaromycin, and its uses and biosynthesis. In particular, a new biosynthetic gene cluster (BGC) has been identified, sequenced and cloned, allowing the BGC to be introduced into and expressed in a host cell to produce the compound. The disclosure and invention accordingly also relates to novel genes and nucleic acid molecules encoding the biosynthetic machinery for the production of nidaromycin, and to constructs, vectors, and host cells for expressing the BGC and methods for producing the compound.

### BACKGROUND

Natural products produced by bacteria and fungi are of huge importance in view of their potential use as pharmaceutical or veterinary products, most notably as antibiotics. The actinomycetes, a class of filamentous Gram-positive bacteria of high GC-content, produce the vast majority of all known antibiotics of microbial origin, and of these, around a half are obtained from the genus Streptomyces.

The genes for synthesis of secondary metabolites such as antibiotics in actinomycetes tend to be organised in clusters, comprising genes encoding biosynthetic enzymes, transporter proteins, and other proteins involving in the synthesis or regulation thereof. Various gene clusters for the synthesis of a number of antibiotics in different organisms have been reported.

The increase of antibiotic resistance in a wide range of bacterial pathogens is a major global health concern, and at the same time the discovery of new anti-microbials has been declining. This has prompted the development of new strategies for antibiotic discovery, and one such approach is the genome mining of actinomycetes to identify novel secondary metabolite gene clusters of unknown function which might potentially encode the genes for biosynthesis of clinically useful antibiotics. There are various strategies for this, including trying to induce the expression of cryptic gene clusters in the natural host by culturing in different conditions etc., or heterologous expression of gene clusters in a surrogate host. We have adopted the latter approach, combined with bioinformatic and phylogenetic analysis, to identify new BGCs encoding the biosynthesis of new antibiotic compounds.

### SUMMARY

To this end, we have performed an analysis of the genomes of 1200 marine actinobacteria strains isolated from the Trondheim Fjord and 576 actinobacteria-type strains retrieved from public databases based on various criteria including phylogenetic novelty and gene cluster diversity, as well as anti-bacterial activity of the strains. Based on this, various strains were selected for further study, including the actinobacteria strain P08-G05. The genome of this strain was sequenced and subjected to bioinformatic analysis to identify and analyse potential new BGCs comprising genes encoding the synthesis of antibiotic-like compounds. This led to the identification of a novel BGC, termed P08-G05-cluster 16 (P08-G05-c16) herein, the DNA sequence of which is shown in SEQ ID NO. 1. The cluster has been cloned, and expressed in a heterologous actinomycete host, specifically in the strain Streptomyces coelicolor M1152ΔmatAB, which is a modified derivative of the model strain Streptomyces coelicolor A3 (2)/M145 (ATCC BAA-471), the preparation of which is described in the Examples below. The P08-G05-c16 gene cluster was cloned in an inducible Bacterial Artificial Chromosome (BAC) vector and transferred into the Streptomyces coelicolor M1152ΔmatAB strain by tri-parental conjugation to prepare the transconjugant strain M1152ΔmatAB (P08-G05_C16). The transconjugant expresses the BGC and synthesises the compound; extracts have been shown to possess antibacterial activity. The novel compound, which we have termed nidaromycin, has been extracted from the heterologous host, purified and subjected to structural analysis and characterisation, which confirms its structural novelty, antibiotic activity, and low cytotoxicity.

Accordingly, in a first aspect provided herein is a compound of Formula (I):

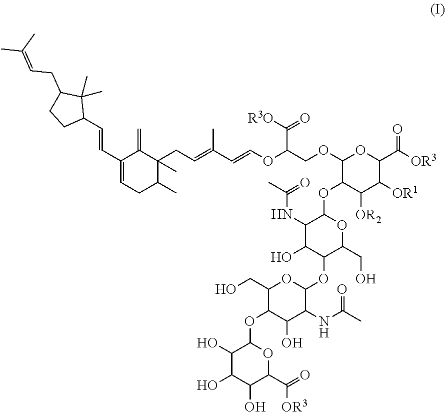

wherein R¹ is —SO₂OH, —SO₂OR or —SO₂R and R² is H, or wherein R² is —SO₂OH, —SO₂OR or —SO₂R and R¹ is H;
wherein R is a C₁-C₂₀ hydrocarbyl group; and
wherein each R³ is independently selected from H or a C₁-C₂₀ hydrocarbyl group;
or a pharmaceutically acceptable salt, solvate, hydrate or ester thereof.

In a second aspect provided herein is a compound of Formula I as defined herein for use as a medicament, or in other words, for use in therapy.

In particular, the medicament is an antibiotic, and the therapy is anti-microbial therapy, particularly anti-bacterial therapy.

Thus, a third aspect provides a compound of Formula I as defined herein for use as an antibiotic medicament, or for use in the treatment of a microbial infection, particularly a bacterial infection.

A fourth aspect provides use of a compound of Formula I as defined herein for the preparation of a medicament for use in the treatment of a microbial infection, particularly a bacterial infection.

A fifth aspect provides a method of treatment of a microbial infection, particularly a bacterial infection in a subject, comprising administering to the subject an effective amount of a compound of Formula I as defined herein.

The subject may be any human or non-human animal, particularly a mammalian animal. Thus, the medical uses herein comprise human clinical and veterinary uses, as well as uses in animal husbandry and agriculture, including use in aquaculture and as a plant protection agent against plant pathogens.

In an embodiment, the bacterial infection is an infection by Gram-positive bacteria.

A sixth aspect provides a pharmaceutical composition comprising a compound of Formula I as defined herein, together with at least one pharmaceutically acceptable carrier, additive and/or excipient. In an embodiment the pharmaceutical composition is suitable for parenteral, oral or topical administration.

As well as the medical uses outlined above, the compound may also have non-medical (i.e. non-therapeutic) uses, employing its anti-microbial/anti-bacterial properties in vitro or ex vivo, for example to decontaminate, disinfect or sterilise surfaces etc.

Accordingly, a seventh aspect provides use of a compound of Formula I as defined herein as an anti-microbial, particularly anti-bacterial, agent.

Expressed in other words, this aspect also provides a method of controlling bacteria on a surface comprising a step of applying to the surface (or contacting the surface with) a compound as defined herein. Controlling bacteria includes inhibiting the growth and/or viability of the bacteria. This may further include reducing the number of the bacteria (e.g. killing the bacteria), and or reducing or preventing their multiplication (replication).

The compound may be prepared by biosynthesis in a host which has been modified, or engineered, to express the BCG which comprises the biosynthetic genes for its synthesis, or in other words a host into which the BGC, or more particularly a nucleic acid molecule comprising the BGC, or the component genes thereof, has been introduced. In an embodiment the host is a heterologous host, that is a host which does not naturally contain the BGC. However, in another embodiment the BGC may be introduced into the organism from which the BGC was obtained, namely the isolate P08-G05, or more generally a strain which endogenously comprises the BGC. Alternatively, the compound may be prepared in an in vitro transcription and translation (IVTT) system.

An eighth aspect thus provides a nucleic acid molecule comprising:
(a) a nucleotide sequence as shown in SEQ ID NO. 1; or
(b) a nucleotide sequence which is the complement of SEQ ID NO. 1; or
(c) a nucleotide sequence which is degenerate with SEQ ID NO. 1; or
(d) a nucleotide sequence having at least 85% sequence identity with SEQ ID NO. 1; or
(e) a part of any one of (a) to (d),
wherein said nucleic acid molecule encodes or is complementary to a nucleic acid molecule encoding one or more polypeptides, or comprises or is complementary to a nucleic acid molecule comprising one or more genetic elements, having functional activity in the synthesis of an antibiotic compound.

The functional activity may be enzymatic activity, or transport or transfer activity, or regulatory activity (e.g. regulation of gene expression), or any other activity which contributes to synthesis or transport of the compound, or component moieties thereof.

Thus, more generally the nucleic acid molecule may be defined as comprising one or more nucleotide sequences which contribute to the biosynthesis of the compound. In other words, the nucleic acid molecule may comprise one or more nucleotide sequences which make up or are part of the biosynthetic gene cluster for synthesis of the compound.

In particular, the antibiotic compound is a compound of Formula I as defined herein, or a derivative thereof.

In particular, in part (e), the part of the nucleotide sequence comprises a sequence which corresponds to a biosynthetic gene or an open reading frame (ORF) encoding a protein involved in the biosynthesis of the compound, or is complementary to or degenerate with such a sequence.

In an embodiment the nucleic acid molecule comprises a nucleotide sequence (a) to (d) and encodes polypeptides for the synthesis of an antibiotic compound, and particularly for the synthesis of a compound of Formula I as defined herein, or a derivative thereof. In other words, the nucleic acid molecule comprises nucleotide sequences which together provide biosynthetic machinery for production of the compound. Accordingly, in this embodiment the nucleic acid molecule may be defined as encoding a biosynthetic system for synthesis of the compound, or as comprising a BGC for synthesis of the compound.

In an embodiment, the nucleic acid molecule comprises nucleotide sequences for the production of the compound in an actinomycete host, particularly a Streptomyces host, more particularly a Streptomyces coelicolor host, especially in a host strain which is Streptomyces coelicolor strain A3 (2), M145, M1152, or M1152ΔmatAB as defined or described herein.

As specified in part (e) the nucleic acid molecule may also encode a part of the complete biosynthetic system, e.g. an individual component of the BGC. Individual genes, or ORFs, in the BGC have been identified and annotated, as described in more detail below (see Table 1). A part of the nucleic acid molecule may thus represent, or correspond to, an individual gene or ORF, for example encoding a polypeptide involved in the biosynthesis of the compound, or two or more such genes or ORFs. Accordingly, in an embodiment, the nucleic acid molecule comprises a nucleotide sequence as shown in any one or more of SEQ ID NOs 2-29, or a nucleotide sequence which is complementary thereto or degenerate thereto, or which has at least 85% sequence identity therewith.

In another embodiment the nucleic acid molecule comprises a nucleotide sequence which encodes an amino acid sequence as shown in any one or more of SEQ ID NOs 30-57, or an amino acid which has at least 85% sequence identity therewith.

A ninth aspect provides a polypeptide encoded by a nucleic acid molecule as defined above.

A tenth aspect provides a recombinant construct comprising a nucleic acid molecule as defined herein.

In an embodiment the recombinant construct comprises one or more other nucleic acid sequences, for example a regulatory sequence, an expression control sequence, or a genetic element involved in replication or transfer of the nucleic acid molecule.

An eleventh aspect provides a vector comprising the nucleic acid molecule or recombinant construct as defined herein.

In an embodiment the vector is a plasmid, cosmid, or artificial chromosome, particularly a bacterial artificial chromosome (BAC).

A twelfth aspect provides a microbial host cell comprising a nucleic acid molecule, recombinant construct, or vector as defined herein. In other words, this aspect provides a modified, or engineered, microbial host cell into which the nucleic acid molecule, recombinant construct, or vector has been introduced. As noted above the host may be a heterologous host. A heterologous host, or modified host cell, does not, by definition, include the natural producer of the compound or the natural strain which endogenously contains the BGC. However, as also noted above, it is not precluded that nucleic acid molecule, recombinant construct or vector is introduced into the original strain from which the nucleic acid molecule was derived. The nucleic acid molecule may be introduced into the host cell in increased copy number, i.e. one or more copies may be introduced.

The host cell may be a production host cell, for production of the compound, or it may be a host cell generated for the purpose of cloning the nucleic acid molecule (e.g. propagating, or producing the nucleic acid molecule), or for transferring it to another host cell (i.e. it may be a cloning or transfer host cell).

A thirteenth aspect provides a method of producing a compound of Formula I as defined herein, said method comprising introducing into a microbial host cell, a nucleic acid molecule, recombinant construct, or vector as defined herein, and allowing the nucleic acid molecule to be expressed (in particular allowing the individual gene sequences, or ORFs, of the nucleic acid molecule to be expressed, i.e. allowing the genes of the BGC to be expressed).

Alternatively defined this aspect provides a method of producing a compound of Formula I as defined herein, said method comprising introducing into a microbial host cell, a nucleic acid molecule, recombinant construct, or vector as defined herein, and culturing the host cell (or allowing it to grow) under conditions in which the biosynthetic system for synthesis of the compound is expressed.

More particularly, the conditions are conditions which allow the compound to be synthesised by the expressed biosynthetic system.

The production method may further comprise the step of recovering the compound (or in other words, collecting or harvesting the compound). Further, the method may comprise the step of isolating, separating or purifying the compound. In an embodiment the host cell is a bacterial host cell, e.g. from the actinobacteria. In a particular embodiment the host cell is an actinomycete, particularly a Streptomyces host cell, more particularly Streptomyces coelicolor, especially Streptomyces coelicolor strain A3 (2), M145, M1152, or M1152ΔmatAB as defined or described herein.

A fourteenth aspect provides a compound obtained or obtainable by a production method as defined herein. In particular, the compound is obtained or obtainable by a method which comprises expressing the nucleic acid molecule in a heterologous actinomycete host cell, particularly a Streptomyces host cell, more particularly Streptomyces coelicolor, especially Streptomyces coelicolor strain A3 (2), M145, M1152, or M1152ΔmatAB as defined or described herein.

The compound obtained or obtainable by the method may be used or may be for use in any of the uses or methods set out above, or may be comprised in the pharmaceutical composition. In another words, in any of the aspects set out above, the compound may alternatively or additionally be defined as a compound obtained or obtainable by a production method as defined herein.

Further, the production methods herein allow the biosynthetic system for synthesis to be modified in order to modify the compound which is produced. Thus, the nucleic acid molecule, or the individual nucleotide sequences comprised therein, which encode biosynthetic enzymes, may be modified, or inactivated or deleted, and/or additional enzyme activities may be introduced. Such modifications may alter the biosynthetic pathway, and allow modified derivatives of the compound to be obtained.

Accordingly, a fifteenth aspect provides a method for preparing a nucleic acid molecule encoding a modified biosynthetic system for synthesis of a modified derivative of a compound of Formula I as defined herein, said method comprising modifying a nucleic acid molecule as defined herein. The nucleic acid molecule may be modified by introducing, mutating, deleting, replacing or inactivating a sequence encoding one or more activities or proteins encoded by said nucleic acid molecule.

In an embodiment one or more of the nucleotide sequences of SEQ ID NOs. 2-29, or a nucleotide sequence which is complementary to or degenerate with any one of SEQ ID NOs. 2-29, is modified.

### DETAILED DESCRIPTION

The disclosure and invention herein relates to a new antibiotic compound, nidaromycin, having the structure set out in Formula I, as defined above. As indicated above, this novel compound was discovered by screening for and identifying new biosynthetic gene clusters in strains of actinobacteria. Prior bioactivity data for the strains, coupled with extensive bioinformatics and phylogenetic analysis efforts, including genome sequencing, gene annotation, cluster analysis and manual curation of the analysis results, as detailed in the Examples below, allowed us to select certain strains and identify novel BGCs in their genomes. Based on our analysis efforts, one such cluster was selected, cluster 16 from the isolate identified as strain P08-G05. This cluster was hypothesised, based on the bioinformatic and cluster analysis, to code for a moenomycin-like novel compound. The cluster was cloned and transferred into a heterologous host, specifically strain Streptomyces coelicolor M1152ΔmatAB, the preparation of which is detailed further below. The resulting transconjugant strain, strain M1152ΔmatAB (P08-G05_C₁₆), was cultivated and antibacterial activity was demonstrated in cell-free extracts against various Gram-positive bacteria, including Staphyloccus aureus and Enterococcus faecium. Further, in the toxicity tests described in the Examples below the compound has been demonstrated not to exhibit toxicity against mammalian cell lines. The compound was purified and subjected to structural elucidation studies, as detailed in the Examples below. This led to the elucidation of the structure of the novel compound.

As indicated in Formula I, we believe that there are 2 possibilities for the position of the sulphate group in this molecule, indicated as R¹ and R² at C4 and C3 respectively of the ring moiety D (see below), referred to as nidaromycin D4 and nidaromycin D3 respectively.

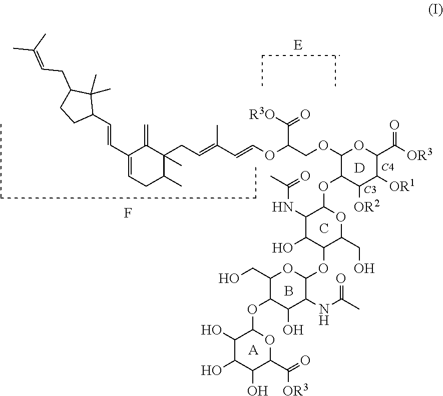

In the compound of Formula (I),
R¹ is —SO₂OH, —SO₂OR or —SO₂R and R² is H, or
R² is —SO₂OH, —SO₂OR or —SO₂R and R¹ is H;
wherein R is a C₁-C₂₀ hydrocarbyl group.

Preferably, R¹ is —SO₂OH or —SO₂OR and R² is H, or R² is —SO₂OH or —SO₂OR and R¹ is H. Most preferably, R¹ is —SO₂OH and R² is H, or R² is —SO₂OH and R¹ is H. It will be appreciated that where the compound is in salt form, the H on any-SO₂OH group can be replaced with a non-H cation.

The carboxylic acid groups in the nidaromycin compound can be modified to their ester form, as indicated by the group —COOR³ in Formula I wherein R³ is C₁-C₂₀ hydrocarbyl. This may be achieved both chemically and enzymatically, as is well known in the art.

By hydrocarbyl is herein typically meant alkyl, alkenyl, alkynyl, or aryl, preferably alkyl and aryl, most preferably alkyl. By C₁-C₂₀ hydrocarbyl is herein typically meant C₁-C₂₀ alkyl, C₁-C₂₀ alkenyl, C₁-C₂₀ alkynyl, or C₆-C₂₀ aryl groups. The alkyl, alkenyl, alkynyl groups may be cyclic or acyclic. The alkyl, alkenyl, alkynyl groups may also be linear or branched. The C₁-C₂₀ hydrocarbyl group is typically a C₁-C₁₀ hydrocarbyl group (e.g. a C₁-C₁₀ alkyl, alkenyl, alkynyl, or aryl group), e.g. a C₁-C₆ hydrocarbyl group (e.g. a C₁-C₆ alkyl, alkenyl, alkynyl, or aryl group), most preferably a C₁-C₆ alkyl group. The above definitions for hydrocarbyl apply to R as well as R³ in particular.

Each R³ group may independently be selected from H or a C₁-C₂₀ hydrocarbyl group. The compound may have all R³ groups as H, two R³ groups as H and the other as C₁-C₂₀ hydrocarbyl, one R³ group as H and the other two as C₁-C₂₀ hydrocarbyl, or three R³ groups as C₁-C₂₀ hydrocarbyl. Preferably, all R³ groups are H. Where the compound is in salt form, the H (i.e. R³ as H) may obviously be replaced with a non-H cation.

Accordingly, in one embodiment, the compound has the structure of Formula II:

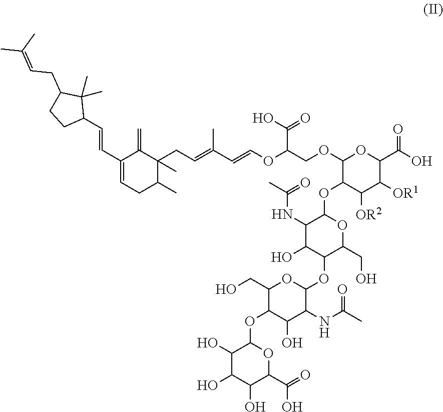

wherein R¹ is —SO₂OH, —SO₂OR or —SO₂R and R² is H, or wherein R² is —SO₂OH, —SO₂OR or —SO₂R and R¹ is H;
wherein R is a C₁-C₂₀ hydrocarbyl group, preferably wherein R¹ is —SO₂OH and R² is H, or wherein R² is —SO₂OH and R¹ is H;
or a pharmaceutically acceptable salt, solvate or hydrate thereof.

In a specific embodiment the compound has the structure I:

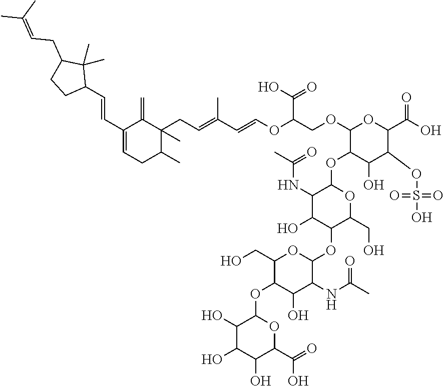 [SMILES: C=C1C(/C=C/C2CCC(CC=C(C)C)C2(C)C)=CCC(C)C1(C)C/C=C(C)/C=C/OC(COC1OC(C(=O)O)C(OS(=O)(=O)O)C(O)C1OC1OC(CO)C(OC2OC(CO)C(OC3OC(C(=O)O)C(O)C(O)C3O)C(O)C2NC(C)=O)C(O)C1NC(C)=O)C(=O)O]

or a pharmaceutically acceptable salt, solvate or hydrate thereof.

In another specific embodiment the compound has the structure II:

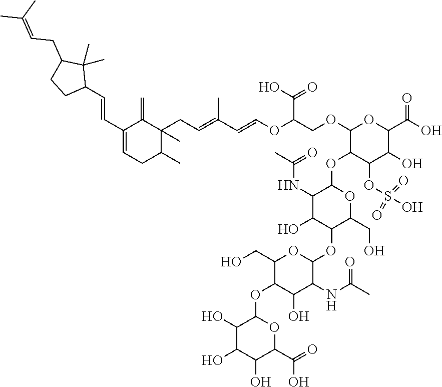 [SMILES: C=C1C(/C=C/C2CCC(CC=C(C)C)C2(C)C)=CCC(C)C1(C)C/C=C(C)/C=C/OC(COC1OC(C(=O)O)C(O)C(OS(=O)(=O)O)C1OC1OC(CO)C(OC2OC(CO)C(OC3OC(C(=O)O)C(O)C(O)C3O)C(O)C2NC(C)=O)C(O)C1NC(C)=O)C(=O)O]

or a pharmaceutically acceptable salt, solvate, or hydrate thereof.

It will be appreciated that the compounds can be in a pharmaceutically acceptable salt, solvate or hydrate form.

The compound may be in the form of a metal salt, e.g. lithium, sodium, potassium or calcium salt (typically one or more R³ group is lithium, sodium or potassium in this case).

A pharmaceutical acceptable salt may also be readily prepared by using a desired acid. The salt may precipitate from solution and be collected by filtration or may be recovered by evaporation of a solvent. Suitable addition salts are formed from inorganic or organic acids which form non-toxic salts and examples are hydrochloride, hydrobromide, hydroiodide, sulphate, bisulphate, nitrate, phosphate, hydrogen phosphate, acetate, trifluoroacetate, maleate, malate, fumarate, lactate, tartrate, citrate, formate, gluconate, succinate, pyruvate, oxalate, oxaloacetate, trifluoroacetate, saccharate, benzoate, alkyl or aryl sulphonates (e.g. methanesulphonate, ethanesulphonate, benzenesulphonate or p-toluenesulphonate) and isethionate.

Those skilled in the art of organic chemistry will appreciate that many organic compounds can form complexes with solvents in which they are reacted or from which they are precipitated or crystallized. These complexes are known as “solvates”. A complex with water is known as a “hydrate”. Solvates and hydrates of the compounds of the invention are within the scope of the invention. The salts of the compounds may form solvates and hydrates and the invention also includes all such solvates and hydrates.

As noted above, the compound has been shown to have antibiotic activity. In other words, the compound has anti-microbial activity. More particularly, the compound has anti-bacterial activity. That is, the compound is capable of inhibiting the growth and/or viability of microorganisms, and particularly bacteria. In an embodiment the compound has anti-bacterial activity against Gram-positive bacteria.

As detailed in the Examples below, extracts of the transconjugant strain producing the compound were tested in a bioassay against a panel of strains and activity was demonstrated. Further, the compound has been purified and activity has been confirmed for the purified compound. Minimal inhibitory concentrations (MICs) have been determined against the Gram-positive indicator organisms as follows:
MIC₇₀ S. aureus ATCC 29213:0.53 μg/ml
MIC₇₀ S. aureus ATCC 43300 (MRSA): 0.53 μg/ml
MIC₇₀: E. faecium CTC 492:8.45 μg/ml
MIC₇₀: E. faecium CCUG 37832:2.11 μg/ml

Other than in the case of E. faecium CCUG 37832, these values compare favourably with vancomycin, which was used as a reference compound. In particular, the efficacy of the compound was higher than vancomycin against both methicillin-resistant and methicillin-susceptible strains of Staphylococcus aureus.

Accordingly, in an embodiment, the compound has activity against drug resistant (or antibiotic resistant) bacteria, particularly multi-drug resistant (MDR) bacteria. Particularly, the compound has activity against drug resistant (or antibiotic-resistant) Gram-positive bacteria, particularly multi-drug resistant (MDR) Gram-positive bacteria.

Particularly, the compound is effective against bacteria, especially Gram-positive bacteria, resistant to methicillin and/or vancomycin.

More generally the compound is effective against especially Gram-positive bacteria, resistant to any class of antibiotics, including antibiotics in the class of β-lactam (including penicillins and cephalosporins), glycopeptide, (phospho)glycolipid, macrolide, tetracycline, sulphonamide, aminoglycoside, carbapenem and quinolone (including fluoroquinolone) antibiotics. In an embodiment, the antibiotic class is β-lactam or glycopeptide.

Particularly, the compound is effective against Staphylococcus and/or Enterococcus. In an embodiment the compound is effective against Staphylococcus aureus and/or Enterococcus faecium. In a more particular embodiment, the compound is effective against methicillin resistant Staphylococcus aureus (MRSA) and/or vancomycin-resistant Enterococcus faecium.

More generally, however, the compound may be used against any species of Gram-positive bacteria, particularly clinically relevant Gram-positive bacteria, including for example, Micrococcus, Streptococcus, Pneumococcus, Bacillus, Listeria, Clostridium.

Antibiotic, or anti-microbial, activity may readily be assessed according to methods well known in the art. For example, broth microdilution assays for determining MICs such as used in the Examples below are widely used and reported, as are agar plate-based method (disc diffusion assays etc.).

Significantly, the compound has also been demonstrated to have no or negligible cytotoxicity against mammalian cells, indicating that it is suitable for clinical use.

Accordingly, as indicated above, the compound herein has medical uses, notably as a therapeutic antimicrobial, or more particularly anti-bacterial, agent, i.e. in the treatment or prevention of microbial, or bacterial, infections.

The subject to be treated with the compound may be any subject suffering, or at risk from, the infection. As noted above the subject is typically a human, but veterinary uses are included and hence the subject may be any animal, particularly a vertebrate, e.g. an animal selected from mammals, birds, amphibians, fish and reptiles. The compound thus has uses both clinically and in animal husbandry and farming settings, including for example aquaculture. In an embodiment the subject is a mammal. The animal may be a livestock or a domestic animal or an animal of commercial value, including laboratory animals or an animal in a zoo or game park. Representative animals therefore include dogs, cats, rabbits, mice, guinea pigs, hamsters, horses, pigs, sheep, goats, cows, chickens, turkeys, guinea fowl, ducks, geese, parrots, budgerigars, pigeons, salmon, trout, cod, haddock, sea bass and carp. The subject may be viewed as a patient.

As well as uses in the context of animals, the compound may also be used as an anti-microbial (or anti-bacterial) agent in the context of plants, i.e. as a plant protection agent against plant pathogens. Accordingly, the compound has issues in the context of agriculture and horticulture generally, or in other words in the treatment or prevention of infections in plants.

The compound is administered to the subject in an amount effective to treat or prevent the infection. An “effective amount” of the compound is the amount of which is effective to inhibit the growth and/or viability of the microorganism, e.g. bacteria, and/or to provide a clinical benefit to the subject, e.g. to provide a measurable or discernible improvement in the clinical condition of the subject, e.g. in one or more clinical parameters or symptoms of the infection.

The skilled person would easily be able to determine what an effective amount of the compound would be on the basis of routine dose response protocols and, conveniently, the routine techniques for assessing microbial growth inhibition etc., as discussed above.

Suitable doses of compound will vary from subject to subject and can be determined by the physician or veterinary practitioner in accordance with the weight, age and sex of the subject, the severity of the condition, and the mode of administration.

The term “treatment” is used broadly herein to include any therapeutic effect, i.e. any beneficial effect on the condition or in relation to the infection. Thus, not only included is eradication or elimination of the infection, or cure of the subject or infection, but also an improvement in the infection or condition of the subject. Thus included for example, is an improvement in any symptom or sign of the infection or condition, or in any clinically accepted indicator of the infection/condition. Treatment thus includes both curative and palliative therapy, e.g. of a pre-existing or diagnosed infection/condition.

The term “prevention” as used herein refers to any prophylactic or preventative effect. It thus includes delaying, limiting, reducing or preventing the infection or its onset, or one or more symptoms or indications thereof, for example relative to the condition or symptom or indication prior to the prophylactic treatment. Prophylaxis thus explicitly includes both absolute prevention of occurrence or development of the infection, or symptom or indication thereof, and any delay in the onset or development of the infection or symptom or indication, or reduction or limitation on the development or progression of the infection or symptom or indication.

For such use, the compound may be formulated as a pharmaceutical composition. The pharmaceutical composition comprises one or more pharmaceutically acceptable carriers, additives, or excipients.

The pharmaceutical composition may be formulated for administration by any convenient or desired means, for example for parenteral, enteral, oral (notably, peroral), or topical administration, or by inhalation.

Conventional galenic preparations include tablets, pills, powders (e.g. inhalable powders), lozenges, sachets, cachets, elixirs, suspensions, emulsions, solutions, syrups, aerosols (as a solid or in a liquid medium), sprays (e.g. nasal sprays), compositions for use in nebulisers ointments, soft and hard (e.g. gelatin) capsules, suppositories, sterile injectable solutions, sterile packaged powders, and the like.

Examples of suitable carriers, excipients, and diluents are lactose, dextrose, sucrose, sorbitol, mannitol, starches, gum acacia, calcium phosphate, inert alginates, tragacanth, gelatine, calcium silicate, microcrystalline cellulose, polyvinylpyrrolidone, cellulose, water syrup, water, water/ethanol, water/glycol, water/polyethylene, hypertonic salt water, glycol, propylene glycol, methyl cellulose, methylhydroxybenzoates, propyl hydroxybenzoates, talc, magnesium stearate, mineral oil or fatty substances such as hard fat or suitable mixtures thereof. Excipients and diluents of note are mannitol and hypertonic salt water (saline).

The compositions may additionally include additives such as lubricating agents, wetting agents, emulsifying agents, suspending agents, preserving agents, sweetening agents, flavouring agents, and the like. Additional therapeutically active agents may be included in the pharmaceutical compositions.

Parenterally administrable forms, e.g., intravenous solutions, should be sterile and free from physiologically unacceptable agents, and should have low osmolarity to minimize irritation or other adverse effects upon administration and thus solutions should preferably be isotonic or slightly hypertonic, e.g. hypertonic salt water (saline). Suitable vehicles include aqueous vehicles customarily used for administering parenteral solutions such as Sodium Chloride Injection, Ringer's Injection, Dextrose Injection, Dextrose and Sodium Chloride Injection, Lactated Ringer's Injection and other solutions known in the art. The solutions can contain preservatives, antimicrobial agents, buffers and antioxidants conventionally used for parenteral solutions, excipients and other additives which are compatible with the compound and which will not interfere with the manufacture, storage or use of products.

For topical administration the compound can be incorporated into creams, ointments, gels, transdermal patches and the like. The compound can also be incorporated into medical dressings, for example wound dressings e.g. woven (e.g. fabric) dressings or non-woven dressings (e.g. gels or dressings with a gel component).

Further delivery systems include in situ drug delivery systems, for example gels where solid, semi-solid, amorphous or liquid crystalline gel matrices are formed in situ and which may comprise the compound. Such matrices can conveniently be designed to control the release of the compound from the matrix, e.g. release can be delayed and/or sustained over a chosen period of time. Such systems may form gels only upon contact with biological tissues or fluids. Typically, the gels are bioadhesive. Delivery to any body site that can retain or be adapted to retain the pre-gel composition can be targeted by such a delivery technique.

For application to oral, buccal and dental surfaces, for example for oral health care or hygiene purposes, toothpastes, dental gels, dental foams and mouthwashes are mentioned specifically.

Inhalable compositions may, for instance, take the form of inhalable powders, solutions or suspensions. These may include for example propellant-free nebulisable solutions.

The compound may be used in conjunction with other therapeutically active agents, including other antibiotics or anti-microbial agents, for example anti-fungal or anti-viral agents. The agents may be used separately, or together in the same composition, simultaneously or sequentially or separately, e.g. at any desired time interval.

Accordingly, also provided herein is a product, e.g. a kit, comprising the compound as defined herein, together with a second therapeutically active agent, as a combined preparation for separate, sequential or simultaneous use in the treatment of a subject, particularly in the treatment of an infection in the subject.

Other possible therapeutically active agents include immunomodulatory agents, e.g. immunostimulatory agents, for example cytokines or interferons, growth factors, enzymes, mucolytics, analgesics, anti-inflammatory agents, bronchodilators, or steroids etc.

As noted above the compound and second therapeutically active agent may be formulated together in the same composition, or in separate formulations, for administration at the same time, or separately, e.g. according to a defined dosage regime.

In addition to medical uses, the antimicrobial properties of the compound may also be harnessed in non-clinical settings. Thus, in addition to the plant protection uses discussed above, the compound may be used abiotic settings, for example on abiotic (or in other words inanimate) surfaces or in abiotic locations, for the purpose of disinfection or decontamination, or to prevent or reduce bacterial colonisation.

Accordingly, the compound may be used as an anti-bacterial agent against bacteria on any surface. The surface is not limited and includes any surface on which bacteria may occur. Inanimate (or abiotic) surfaces include any such surface which may be exposed to microbial contact or contamination. Thus, particularly included are surfaces on medical equipment, or machinery, e.g. industrial machinery, or any surface exposed to an aquatic environment (e.g. marine equipment, or ships or boats or their parts or components), or any surface exposed to any part of the environment, e.g. pipes or on buildings. Such inanimate surfaces exposed to microbial contact or contamination include in particular any part of: food or drink processing, preparation, storage or dispensing machinery or equipment, air conditioning apparatus, industrial machinery, e.g. in chemical or biotechnological processing plants, storage tanks, medical or surgical equipment and cell and tissue culture equipment. Any apparatus or equipment for carrying or transporting or delivering materials is susceptible to microbial contamination. Such surfaces will include particularly pipes (which term is used broadly herein to include any conduit or line). Representative inanimate or abiotic surfaces include, but are not limited to food processing, storage, dispensing or preparation equipment or surfaces, tanks, conveyors, floors, drains, coolers, freezers, equipment surfaces, walls, valves, belts, pipes, air conditioning conduits, cooling apparatus, food or drink dispensing lines, heat exchangers, boat hulls or any part of a boat's structure that is exposed to water, dental waterlines, oil drilling conduits, contact lenses and storage cases.

As noted above, medical or surgical equipment or devices represent a particular class of surface on which bacterial contamination may form. This may include any kind of line, including catheters (e.g. central venous and urinary catheters), prosthetic devices e.g., heart valves, artificial joints, false teeth, dental crowns, dental caps and soft tissue implants (e.g. breast, buttock and lip implants). Any kind of implantable (or “in-dwelling”) medical device is included (e.g. stents, intrauterine devices, pacemakers, intubation tubes (e.g. endotracheal or tracheostomy tubes), prostheses or prosthetic devices, lines or catheters). An “in-dwelling” medical device may include a device in which any part of it is contained within the body, i.e. the device may be wholly or partly in-dwelling.

The surface can be made of any material. For example, it may be metal, e.g. aluminium, steel, stainless steel, chrome, titanium, iron, alloys thereof, and the like. The surface can also be plastic, glass, brick, tile, ceramic, porcelain, wood, vinyl, linoleum, or carpet, combinations thereof, and the like. The surfaces can also be food, for example, beef, poultry, pork, vegetables, fruits, fish, shellfish, combinations thereof, and the like.

The compound may also be incorporated within materials and products for such disinfection use.

For such in vitro use, the compound may also be used in conjunction with other agents, for example, other anti-microbial agents, disinfectants, cleaning agents etc., or it may be incorporated into paints and coatings etc.

The compound is prepared biosynthetically by expression of the BGC in a suitable host. To this end, provided herein is a nucleic acid molecule comprising a nucleotide sequence corresponding to the BGC (or in other words, a nucleic acid molecule comprising the component nucleotide sequences of the BGC).

The nucleotide sequence of the BGC is set out in SEQ ID NO. 1, which represents the sequence of the BGC as cloned from the marine actinomycete isolate strain PG08-G05. SEQ ID NO. 1 has been annotated and shown to contain a number of genes, or ORFs, which encode the various polypeptides responsible for the activities required for synthesis of the compound.

In particular, the putative functions of the genes were predicted using antiSMASH software, coupled with manual analysis and curation. AntiSMASH is a software package used for identification, annotation and analysis of secondary metabolite biosynthesis gene clusters in microbial genome sequences (Medema et al., Nucleic Acids Research, 2011, 39, Web server issue, W339-W346).

The BGC encodes components necessary for production of the compound in a host strain or IVTT system. However, not all of the encoded polypeptides have yet been ascribed a role in the biosynthesis, and thus not all of the ORFs may be essential. The various genes/ORFs may encode enzymes that catalyse one or more reactions in the biosynthetic pathway, or proteins which do not have enzyme activity but instead are involved in other processes, such as regulation of the process of synthesis, e.g. a transcription factor, or transport, for example of the compound, or an intermediate or substrate compound within, or in or out of the cell, or in conferring resistance (or “immunity”) to the synthesised compound. A number of sequences encoding a transporter protein have been identified. The BGC contains 28 ORFs as set out in Table 1 below.

TABLE 1

| | Coordinates in | | | |
|---|---|---|---|---|
| | GenBank file | | | SEQ |
| Gene | (sequence as | Gene | | ID |
| ID | cloned) | strand | Putative function | NO: |
| ctg4_242 | 2,705 −> 4,399 | + | ABC transporter | 2 |
| ctg4_243 | 4,396 −> 6,255 | + | ABC transporter | 3 |
| ctg4_244 | 6,851 −> 6,243 | − | Protein with S4 domain | 4 |
| ctg4_245 | 6,924 −> 7,658 | + | | 5 |
| ctg4_246 | 9,114 −> 7,813 | − | FAD oxidoreductase | 6 |
| ctg4_247 | 9,923 −> 9,114 | − | | 7 |
| ctg4_248 | 10,722 −> 9,907 | − | Polyprenyl synthetase | 8 |
| ctg4_249 | 11,972 −> 10,707 | − | Glycosyltransferase | 9 |
| ctg4_250 | 14,130 −> 12,001 | − | FAD linked oxidase | 10 |
| ctg4_251 | 14,936 −> 14,100 | − | Radical SAM | 11 |
| | | | superfamily enzyme | |
| ctg4_252 | 16,129 −> 14,933 | − | Glycosyltransferase | 12 |
| ctg4_253 | 17,922 −> 16,147 | − | ABC transporter | 13 |
| ctg4_254 | 19,663 −> 17,924 | − | ABC transporter | 14 |
| ctg4_255 | 20,255 −> 19,668 | − | Isopentenyl diphosphate | 15 |
| | | | isomerase | |
| ctg4_256 | 20,602 −> 21,687 | + | Polyprenyl synthetase | 16 |
| ctg4_257 | 21,732 −> 22,679 | + | NAD-dependent | 17 |
| | | | epimerase/dehydratase | |
| ctg4_258 | 22,679 −> 23,476 | + | Polyprenol- | 18 |
| | | | monophosphomannose | |
| | | | synthase | |
| ctg4_259 | 24,216 −> 23,518 | − | ABC transporter | 19 |
| ctg4_260 | 24,893 −> 24,213 | − | ABC transporter | 20 |
| ctg4_261 | 25,098 −> 25,661 | + | | 21 |
| ctg4_262 | 25,742 −> 26,137 | + | Immunity protein | 22 |
| ctg4_263 | 26,229 −> 26,765 | + | Protein with AAA | 23 |
| | | | domain | |
| ctg4_264 | 27,159 −> 27,737 | + | | 24 |
| ctg4_265 | 28,012 −> 28,692 | + | Chitin binding protein | 25 |
| ctg4_266 | 29,459 −> 28,959 | − | Cellulose binding | 26 |
| | | | protein | |
| ctg4_267 | 29,785 −> 30,813 | + | | 27 |
| ctg4_268 | 31,408 −> 30,944 | − | | 28 |
| ctg4_269 | 32,024 −> 31,383 | − | RNA polymerase | 29 |

The amino acid sequences corresponding to the translations of SEQ ID NOs. 2-29 are shown in SEQ ID NOs. 30-57 respectively.

As noted above, the nucleic acid molecule may comprise nucleotide sequences corresponding to all or part of the BGC. Thus, it may comprise nucleotide sequences corresponding to all 28 of SEQ ID NOs. 2-29 (or of sequences having at least 85% sequence identity therewith), or it may comprise a subset thereof. In other words, it may comprise all or part of SEQ ID NO. 1 (or of a sequence having at least 85% sequence identity therewith). The part may correspond to, or represent an individual ORF, or gene, e.g. it may encode a polypeptide involved in biosynthesis of the compound. Such polypeptides and their coding sequences may represent individual useful products in their own right, and may have other uses beyond the biosynthesis of the compound.

In another embodiment, the part may comprises multiple, or 2 or more ORFs, but less than the full complement of 28 ORFs, i.e. 2-27 of any of SEQ ID NO.s 2-29. The part may comprise ORFs necessary and sufficient for biosynthesis of the compound.

Whilst nucleotide sequences encoding a polypeptide are a particular embodiment herein, in another embodiment the nucleotide sequence may comprise a functional genetic element, such as a promotor, operator, promoter-operator, enhancer, or other regulatory region.

The nucleic acid molecule need not comprise the entire cluster, as indicated in Table 1, but may comprise a portion or part of it. This may comprise one or more genes and/or regulatory sequences, or non-coding or coding functional genetic elements etc. Generally speaking, for production of the compound, a nucleic acid molecule will comprise a number of different genes and/or regulatory molecules leading to the synthesis of an antibiotic compound, e.g. nidaromycin or a derivative thereof. It is also possible to include in the nucleic acid molecule one or more further nucleotides sequences encoding another activity, for example a polypeptide with an activity for modifying the structure of nidaromycin, e.g. to create a derivative or analogue, for example to generate an ester as included in Formula I. As described further below, it is also possible to modify the sequences encoding the enzymes to alter their activity, or to delete or inactivate them, to alter the structure of the compound which is synthesised, to generate a derivative molecule.

In a particular embodiment, the nucleic acid molecule comprises nucleotide sequences sufficient for synthesis of the compound in a suitable host cell. In other words, the nucleic acid molecule encodes a biosynthetic system, or biosynthetic machinery, for synthesis of the compound. Put another way, the nucleic acid molecule comprises a BGC for synthesis of the compound.

The nucleotide sequence comprised in the nucleic acid molecule may be defined as a biosynthetic gene or ORF, that is a gene or ORF which encodes a polypeptide which is functional in the biosynthetic process for a compound of Formula I (or more particularly Formula II), or for nidaromycin (as depicted in Structures I and II above) or a derivative thereof, or a related molecule.

A part of the nucleic acid molecule may in an embodiment be at least 300, e.g. at least 350, 400, 450, 500, 600, 700, 800, 900 or 1000 bases long, notably where individual genes/ORFs are concerned. However, where the molecule comprises sequences corresponding to all or a major part of the BCG, the part will be considerably larger, e.g. at least 10,000, 20,000, 25,000 or 30,000 bases.

The nucleic acid molecule may be an isolated molecule, that is separated from the components with which it is normally found in nature, or it may be a recombinant or synthetic nucleic acid molecule. Generally speaking, since the BGC has been cloned from its natural host, the molecule will be an artificial molecule.

It may be any nucleic acid, but generally speaking it will be DNA.

As defined above, the nucleic acid molecule may comprise nucleotide sequences which are variants of the sequences of SEQ ID NOs. 1-29, or which encode variants of the amino acid sequences of SEQ ID NOs. 30-57, e.g. functionally equivalent variants. Such variants may include parts, degenerate sequences, or homologues defined by a % sequence identity to any one or more of SEQ ID NOs. 1-57. The activity of the variant polypeptides, or the polypeptides encoded by the variant nucleotide sequences may be as defined above.

The term “biosynthetic gene or ORF” as used above includes such variant sequences. The variant sequences retain at least one function of the entity from which they are derived, e.g. encode a polypeptide with substantially the same property or activity of the original/source/parental polypeptide, or at least the same general type of property or function.

In general, the term “gene” includes the ORF which encodes the polypeptide, and may include regulatory sequences such as promoters. The term “ORF” refers only to the part of the gene which is responsible for encoding the polypeptide. As noted above, the nucleic acid molecule may comprise a nucleotide sequence selected from SEQ ID NO.1, or any one or more of SEQ ID NOs. 2-29, or a nucleotide sequence which exhibits at least 85% sequence identity with any aforesaid sequence. More particularly, this may be at least 87, 90, 91, 92, 93, 94, 95, 96, 97, 98, 99 or 100% sequence identity, or a sequence which is complementary or degenerate thereto.

Further, the nucleic acid molecule may comprise a nucleotide sequence encoding one or more amino acid sequences selected from SEQ ID NOs. 30-57, or an amino acid sequence which exhibits at least 85% sequence identity thereto.

Analogously a polypeptide herein, that is a polypeptide encoded by a nucleic acid molecule as defined and described herein, may comprise all or part of an amino acid sequence as set out in any one of SEQ ID NOs. 30-57, or an amino acid sequence having at least 85% sequence identity thereto.

More particularly, in the context of the amino acid sequences indicated above, this may be at least 87, 90, 91, 92, 93, 94, 95, 96, 97, 98, 99 or 100% sequence identity with the amino acid sequence as shown in any one of SEQ ID NOs. 30-57. % sequence identity may readily be determined with the aid of commercially available sequence comparison programs which can calculate percentage homology or identity between two or more sequences.

Percentage homology or sequence identity may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap.

This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum percentage homology/sequence identity therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12:387). Examples of other software that can perform sequence comparisons include, but are not limited to, the BLAST package (see Ausubel et al. (1999) ibid-Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174:247-50; FEMS Microbiol. Lett. (1999) 177:187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62. Suitably, the percentage identity is determined across the entirety of the reference and/or the query sequence. Once the software has produced an optimal alignment, it is possible to calculate percentage homology, preferably percentage sequence identity. The software typically does this as part of the sequence comparison and generates a numerical result.

Such variants of the specified sequences may readily be prepared using recombinant DNA techniques such as site-directed mutagenesis or gene replacement or gene editing techniques, homologous recombination etc. A variety of such methods are known and described in the art.

As noted above, nucleic acid molecules and nucleic acid sequences as defined herein may comprise any nucleic acid, and this may be DNA or RNA. They may be single-stranded or double-stranded. It will be understood by a skilled person that numerous different nucleic acid molecules/nucleotide sequences can encode the same polypeptide as a result of the degeneracy of the genetic code. In addition, it is to be understood that the skilled person may, using routine techniques, make nucleotide substitutions that do not affect the polypeptide sequence encoded by the nucleic acid molecules/polynucleotides/nucleotide sequences as defined herein to reflect the codon usage of any particular host organism in which the polypeptides of the invention are to be expressed.

Nucleic acid molecules/nucleotide sequences such as DNA nucleic acid molecules/sequences may be produced recombinantly, synthetically or by any means available to those of skill in the art. They may also be cloned by standard techniques.

Longer nucleic acid molecules/polynucleotides/nucleotide sequences will generally be produced using recombinant means, for example using polymerase chain reaction (PCR) cloning or other cloning techniques.

The present nucleic acid molecule may further comprise a nucleotide sequence encoding a selectable marker. Suitably selectable markers are well known in the art and include, but are not limited to, fluorescent proteins-such as GFP. Suitably, the selectable marker may be a fluorescent protein, for example GFP, YFP, RFP, tdTomato, dsRed, or variants thereof.

The nucleic acid molecule may be provided as part of a nucleic acid construct, which comprises the nucleic acid molecule together with one or more other nucleotide sequences. These other nucleotide sequences may encode a selectable marker or other polypeptide, which may be any other polypeptide it is desired to introduce into a host cell along with the BGC.

In another embodiment the nucleic acid molecule may be provided in the form of a recombinant construct comprising the nucleic acid molecule operably linked to one or more expression control sequence, for example a promoter, optionally with one or more further regulatory sequences. Thus, for example nucleotide sequences corresponding to individual or selected ORFs from the BGC may be provided in the construct with heterologous regulatory sequences for control of gene expression. A construct comprising a nucleic acid molecule comprising one or more coding sequences and one or more expression control sequences may be referred to herein as an expression construct.

The nucleic acid molecule or recombinant construct may be comprised within a vector, which may be for the purposes of cloning, transfer or for expression. Thus, in embodiments, the vector may be a cloning vector, transfer vector or expression vector. As used herein, the term “vector” refers to any genetic element capable of serving as a vehicle of genetic transfer, expression, or replication for an exogenous nucleic acid sequence in a host strain.

A vector may exist as a single nucleic acid molecule or as two or more separate nucleic acid molecules. Vectors may be single copy vectors or multicopy vectors when present in a host strain.

A particular vector for use herein is an expression vector. In such a vector, one or more gene/coding sequences can be inserted into the vector molecule, in proper orientation and proximity to expression control elements so as to direct expression of one or more proteins when the vector molecule is present in the host strain. The expression control elements may be provided by the vector, but conveniently they are part of the nucleic acid molecule which is inserted into the vector (i.e. the nucleic acid molecule derived from the BGC as defined and described herein), particularly where the nucleic acid molecule comprises nucleotide sequences corresponding to the complete or substantially complete BGC, or a major part thereof. In other words, in an embodiment, the nucleic acid molecule in the vector may comprise control and regulatory sequences for expression of the coding nucleotide sequences, and the vector may simply be a vehicle for the molecule, for its cloning, or introduction into a cell, reproduction in the cell etc.

Construction of appropriate vectors and other recombinant or genetic modification techniques for use herein are well known in the art (see, e.g., Green and Sambrook, Molecular Cloning, A Laboratory Manual, Cold Spring Harbor Laboratory Press (Cold Spring Harbor, N.Y.) (2012), and Ausubel et al., Short Protocols in Molecular Biology, Current Protocols John Wiley and Sons (New Jersey) (2002).

The vector can be a plasmid, cosmid, phagemid or other phage vector, viral vector, episome, an artificial chromosome, e.g. bacterial artificial chromosome (BAC) or P1 artificial chromosome (PAC), or other polynucleotide construct.

Conveniently the vector is an artificial chromosome, particularly a BAC. This is particularly the case where the nucleic acid molecule comprises sequences corresponding to the entire BGC or a substantial or major part thereof, in view of the size of the molecule. As described in the Examples below, the BGC was cloned in a BAC, and generally speaking for cloning of a nucleic acid molecule for synthesis of the compound in a host an artificial chromosome, particularly a BAC, would be used. An exemplary BAC for this purpose is the BAC vector pDualP of Varigen Biosciences, Madison, WI, USA.

For cloning and expression of smaller nucleic acid molecules a wide range of plasmid suitable for use in selected or desired host strains of bacteria are available and known in the art.

Generally, regulatory control sequences are operably linked to the coding nucleic acid sequences, and include constitutive, regulatory and inducible promoters, transcription enhancers, transcription terminators, and the like which are well known in the art. The coding nucleic acid sequences can be operably linked to one common expression control sequence or linked to different expression control sequences. However, as indicated above, conveniently the native control sequences of the genes of the BGC are used, particularly in the case of a vector comprising a nucleic acid molecule for synthesis of the compound.

Suitable promoter sequences for expression in bacteria, particularly actinomycetes, are known in the art. Where heterologous promoters are used, it may be advantageous to use a strong promoter. In particular, a strong inducible promoter may be used. This may be the case for expression of an individual gene or ORF, for example.

The choice of the vector will typically depend on the size of the nucleic acid molecule and the compatibility of the vector with the host strain into which the vector is to be introduced. The vectors may be linear or closed circular plasmids. The vector may also be an autonomously replicating vector, i.e., a vector which exists as an extrachromosomal entity, the replication of which is independent of chromosomal replication, e.g. a plasmid, an extrachromosomal element, a minichromosome, or an artificial chromosome. The vector may contain any means for assuring self-replication. Alternatively, the vector may be one which, when introduced into the host strain, is integrated into the genome and replicated together with the chromosome(s) into which it has been integrated. Integrative plasmids are known in the art. Furthermore, a single vector or plasmid or two or more vectors or plasmids which together contain the total nucleic acid to be introduced into the genome of the host strain, or a transposon may be used.

As noted above, the vectors may contain one or more selectable markers which permit easy selection of transformed cells. The selectable marker genes can, for example, encode detectable products, e.g. fluorescent proteins, provide resistance to antibiotics or toxins, complement auxotrophic deficiencies, or supply critical nutrients not in the culture media, and/or provide for control of chromosomal integration. Examples of bacterial selectable markers are markers which confer antibiotic resistance such as ampicillin, kanamycin, chloramphenicol, apramycin, or tetracycline resistance.

The vectors may also contain one or more elements that permit integration of the vector into the host strain genome or autonomous replication of the vector in the host independent of the genome. For integration into the host strain genome, the vector may rely on an encoding nucleic acid sequence or other element of the vector for integration into the genome by homologous or non-homologous recombination. CRISPR-based systems may also be used to achieve integration. For autonomous replication, the vector may further comprise an origin of replication enabling the vector to replicate autonomously in the strain in question. The origin of replication may be any plasmid replicator mediating autonomous replication which functions in a cell. The term “origin of replication” or “plasmid replicator” is defined herein as a nucleotide sequence that enables a plasmid or vector to replicate in vivo.

The vector may be introduced into the host cell by any convenient or desired means, and this may depend on the nature of the vector. As used herein, the term “introduced” refers to methods for inserting foreign nucleic acid, e.g. DNA or RNA, into a cell. This includes both conjugation and transformation methods, or indeed any method suitable for transferring a nucleic acid molecule or vector into a host cell. A host cell which has been modified by introduction of a nucleic acid molecule or vector may be referred to as an engineered host cell. An engineered host cell is accordingly distinguished from a native or wild-type host cell by the introduction of a nucleic acid molecule into the cell, which is not present in the native or wild-type host cell.

For plasmid vectors and such like, methods of transformation are known in the art. However, for larger vectors, such as would be used to transfer a nucleic acid molecule for synthesis of the compound methods for transfer of the plasmid into the host cell by conjugation would typically be used. This may involve transfer of the vector into an intermediate host, i.e. a transfer host, before introduction into the host for expression, e.g. for production of the compound. Conveniently, to transfer a vector, e.g. BAC, for production of the molecule into the production host, a tri-parental conjugation method may be used, as known and reported in the art. Thus, the vector comprising the nucleic acid molecule for synthesis of the compound may be transferred into the transfer host, together with a driver plasmid, by triparental mating of the cloning host containing the vector, a host containing the driver plasmid, and the transfer host. Such a process is described in the Examples below. Subsequently, the transfer host comprising the vector and driver plasmid is conjugated with the intended production host cell to transfer the vector into the production host cell for synthesis of the compound.

As described above, the vector, once introduced, may be maintained as a chromosomal integrant or as a self-replicating extra-chromosomal vector. The transformation can be confirmed using methods well known in the art. Such methods include, for example, PCR at the integration site (primers in vector and host chromosome) or genome sequencing. Alternatively or additionally, an analysis at the gene expression level may be performed, using for example Northern blots or polymerase chain reaction (PCR) amplification of mRNA, or immunoblotting for expression of gene products, or other suitable analytical methods to test the expression of an introduced nucleic acid sequence or its corresponding gene product. In the case of a vector comprising a nucleic acid molecule for synthesis of the compound, the generation and detection of the compound will confirm that expression has taken place. Expression levels can further be optimized to obtain sufficient expression using methods well known in the art.

The host cell into which the vector is transferred will depend on the purpose of the host cell, i.e. whether it is for cloning or transfer, or expression of one or more polypeptides, or for production (synthesis) of the compound.

A host cell suitable as a cloning host may be any cell know in the art for such a purpose, and will depend on the size of the nucleic acid molecule or vector. For example, for cloning of smaller nucleic acid molecules comprising nucleotide sequences encoding single polypeptides or a smaller selection thereof, a wide range of host cells may be used, including for example various strains of E. coli. Where the nucleic acid molecule is a large molecule, particularly for synthesis of the compound, the host cell will need to be suitable for propagation of large constructs, and again such hosts are known in the art, including for example, E. coli strain 10Beta.

Suitable hosts for transfer by conjugation are also known in the art, and include for example E. coli ET12567.

Suitable hosts for expression of individual polypeptides are also known in the art and include many strains of E. coli, Bacillus and other bacteria, including actinomycetes and particularly Streptomyces.

For production of the compound, the host cell may be any suitable host cell, in which the nucleic acid molecule may be expressed and the compound synthesised. The production host will typically be bacteria, conveniently an actinomycete, and more particularly bacteria of the genus Streptomyces.

The production host cell may be a heterologous host cell, that is a host cell which does not natively contain the BGC, or synthesise the compound. However, in an alternative embodiment, the nucleic acid molecule may be introduced into the organism from which the BGC was cloned, namely the isolate P08-G05, or more generally a strain which endogenously comprises the BGC.

A range of different Streptomyces hosts are known in the art and available for use. In an embodiment the host is Streptomyces coelicolor. Again, various strains and isolates of S. coelicolor are available for use. The strain A (3) 2 is a well-known model strain.

Streptomyces coelicolor strain M145, also known as Streptomyces violaceoruber (Waksman and Curtis) Pridham, is a prototrophic derivative of strain A(3)2 and is available from the ATCC under number BAA-471. Streptomyces coelicolor strain M145 (ATCC BAA-471) is described in Bentley et al., 2002, Nature, 417, 141-147, and in particular lacks the plasmids SCP1 and SCP2 of the parent A3 (2) strain. Derivatives of Streptomyces coelicolor strain M145 (ATCC BAA-471) have been engineered for use in heterologous expression secondary metabolite gene clusters, as described in Gomez-Escribano and Bibb, Microbial Biotechnology, 2011, 4 (2), 207-215. Any of the strains described in this document may be used. The strains are modified to delete all or some of the four antibiotic gene clusters (act, red, cda and cpk gene clusters) in strain M1146, and further to introduce point mutations in rpoB and/or rpsL, the genes that encode the RNA polymerase β-subunit and ribosomal protein S12 respectively. Each mutation has been shown to enhance levels of antibiotic production in Streptomyces without growth impairment. The mutant alleles were incorporated into a suicide plasmid and substituted for the wild-type genes by homologous recombination, as described in the document above. Any of strains M1141-M1146 or M1151-M1156 as described in this document may be used. Particular mention may be made of strain M1152 (Aact Ared Acpk Acda rpoB (C₁₂₉₈T)). Further, as described in the Examples below, mutants of strain M1152 have been generated, specifically in-frame deletion mutants for SCO2963 and SCO2962, in which the matAB locus has been deleted. Strain Streptomyces coelicolor M1152ΔmatAB as described in Example 1 below represents a preferred host cell for production of the compound.

Once the nucleic acid molecule has been introduced into the host strain for production of the compound, the modified bacteria are grown, or cultured, under conditions suitable for expression of the encoded polypeptides and for synthesis of the compound. Once again, procedures and conditions for this are known in the art, and this can readily be achieved according to techniques and principles well known in the art.

Growth media suitable for Streptomyces bacteria are known in the art and described in the Examples below, for example, MG-2.5 w/NaCl medium.

Alternatively, the compound may be prepared in an in vitro transcription and translation (IVTT) system, i.e. a cell-free system, according to principles and techniques known in the art. This may include cell extracts of the host cells described above, including from the various S. coelicolor strains discussed above, or Streptomyces or actinomycete cells more generally.

After the compound has been synthesised, or a desired polypeptide has been expressed, it may be harvested, or in other words recovered, or collected from the culture.

In particular, the compound or the polypeptide may be extracted or separated from bacterial cells by cell lysis procedures, again well known in the art. Thus, crude extracts may be obtained, which contain the compound or polypeptide.

Further, separation and purification procedures known in the art may be used to isolate, or purify the compound or polypeptide. These include for example, precipitation, chromatography or filtration methods, e.g. ammonium sulphate precipitation, ion exchange chromatography, reverse phase chromatography, size exclusion chromatography, gel filtration, HPLC methods and such like. Any desired or convenient combination of purification methods may be used. For isolation of the compound, extraction with solvents and/or acids and preparative chromatography methods, e.g. HPLC, may be used. A suitable procedure is described in the Examples below.

Thus, the methods herein may comprise a further step of purifying the compound or polypeptide.

In a particular aspect and embodiment herein the compound is a compound obtainable by expression of a nucleic acid molecule comprising SEQ ID NO. 1 in Streptomyces coelicolor strain M1152ΔmatAB. More particularly, the compound is obtainable by expression of a nucleic acid molecule comprising SEQ ID NO.1 in Streptomyces coelicolor strain M1152ΔmatAB according to the methods described in the Examples below.

Thus, the compound may have the structure shown in Formula I above, which includes more particularly the structure of Formula II or structure I or II above. However, as indicated above, modifications of the compound may be obtained, i.e, derivatives may be generated, either by chemical modification of the compound, or by modifying one or more the of the coding sequences of the nucleic acid molecule. Such modification may alter one or more enzyme activities, which results in modification of the resulting compound which is synthesised. The modification of the genes of antibiotic gene clusters to modify the antibiotic compound which is produced has been described in the art, for example in WO 2001/059126 (nystatin) or WO 2009/115822 (BE-14106).

The invention will now be described in more detail in the following non-limiting examples with reference to the following figures.

### BRIEF DESCRIPTION OF FIGURES

FIG. 1 shows LC-DAD-isoplots of extracts of M1152ΔmatAB (P08-G05_C16) (A) and the control M1152ΔmatAB (B) cultivated in well plate with 2.5×MG-2.5w/NaCl and 0.1% inducer-caprolactame. The two compounds eluting between 13 and 15 min are only observed in the extract of the transconjugant, Nidaromycin eluting at 14.6 min and a derivative eluting at 13.2 min.

FIG. 2 shows MS spectrum of the compound produced by the transconjugant P08-g05_C16 (corresponding to the main UV peak observed in extracts of the transconjugant). The compound also forms Na+ adducts in the MS. The red bars show the theoretical isotopic distribution of the proposed molecular formula, whereas the black bars show the measured isotopic distribution of the compound;

FIG. 3 shows a LC-DAD isoplot of the HPLC purified compound produced by the transconjugant M1152ΔmatAB (P08-G05_C16). Nidaromycin is eluting as a single peak at approximately 16 min in this chromatogram, and no other UV absorbing compounds are observed in the chromatogram.

FIG. 4 shows toxicity data for the compound nidaromycin, as produced by the transconjugant M1152ΔmatAB (P08-G05_C16) as described in Example 2, against the cell lines HepG and LLC-PK1 at varying concentrations from 0.5 to 50 μg/ml (A) viability after 48 hrs exposure expressed as % and (B) LDH leakage (%) after 48 hrs exposure;

FIG. 5 shows MS spectra of ¹⁵N labelled nidaromycin (A) and ¹³C and ¹⁵N labelled nidaromycin (B) showing that the molecular weight increases with 2 Da and 63 Da respectively which demonstrate that the formula contains 2N and 61C.

FIG. 6 shows MSMS fragmentation pattern of purified nidaromycin. The precursor mass is M+H+1349.5674

FIG. 7 shows the predicted structure of the compound produced by the transconjugant M1152ΔmatAB (P08-G05_C16) as determined by NMR studies in Example 8, including atom numbering for atom-specific assignments in NMR spectra used in the Example.

### EXAMPLES

### Example 1

Preparation of Host Strain Streptomyces coelicolor M1152ΔmatAB

S. coelicolor strain M1152 as described in Gomez-Escribano 2010 (supra) was obtained from the John Innes Centre, Norwich, UK.

In-frame deletion mutants for SCO2963/SCO2962 in S. coelicolor M1152 were created as described earlier (van Dissel et al., 2015. Microbial Cell Factories, 14 (1), pp. 1-10). In brief, the upstream region of SCO2963 ranging from 1326 to +43 relative to the start codon and the downstream region of SCO2962 from +2190 to +3610 were amplified by PCR from the S. coelicolor genome using the primers listed in Table 2. The amplified flanks were cloned into the unstable shuttle vector pWHM3-onT (Wu et al., 2019. Angewandte Chemie, 131 (9), pp.2835-2840) using the EcoRI and HindIll restriction site. The Xbal site, featured in both amplified regions, was used for insertion of the apramycin resistance cassette aacC4 flanked by loxP sites between the flanking regions. The completed vector (pMAT1) was introduced into E. coli ET12567+PUZ8002, which allowed transfer of pMAT1 towards S. coelicolor M1152 by conjugation. Mutants where the matAB locus was replaced by the aacC4 cassette and where the pWHM3 vector was lost were selected by replicate plating for a Thio−/Apra+phenotype. A marker free S. coelicolor M1152 ΔmatAB strain was obtained by introduction of the pUWLcre plasmid, expressing the Cre recombinase, which incised the loxP sites surrounding the apramycin resistance gene.

TABLE 2

| | |
|---|---|
| matA_−1326 | AGTCGAATTCCAGCCGGGCGGTGAGATTCC |
| matA_+43 | ACTGTCTAGACGAGCACTCGTCGGCCGAAC |
| matB_+2190 | AGTCTCTAGAAGGCCGGTCGGATGACCACC |
| matB_+3610 | AGTCAAGCTTCCCTGTTCACTCCCGCAACCG |

### Example 2

Identification of the Biosynthetic Gene Cluster (Cluster 16) from Marine Actinobacteria Isolate P08-G05
Origin of marine isolate strain P08-G05

The marine isolate P08-G05 was obtained from the SINTEF/NTNU marine Actinobacteria strain collection, which was built from water, sediment, and sponge samples taken from the Trondheim fjord. The strain was selected based on a comprehensive assessment of the draft genomes of 1200 isolates from the strain collection based on different criteria: phylogenetic novelty, gene cluster diversity, and previously observed bioactivity, as described below.

The frozen glycerol culture from the collection was streaked on TSA (Trypton soya broth agar) supplemented with 0.5× artificial sea water (Engelhardt et al. 2010, Applied and Environmental Microbiology 76 (15): 4969-4976.). A pure isolate was cultivated in TSB with artificial sea water to produce mycelia for a working cell bank.

### Illumina Sequencing and De Novo Assembly of the Genome of Strain P08-G05

Biomass of strain P08-G05 for genome sequencing was produced in TSB medium supplemented with 50% artificial sea water at 30° C. The biomass was collected by centrifugation and sent for sequencing to BaseClear BV, where the extraction of DNA, sequencing, and post-sequencing data processing were carried out.

Paired-end sequence reads were generated using the Illumina HiSeq2500 system. FASTQ sequence files were generated using the Illumina Casava pipeline version 1.8.3. Initial quality assessment was based on data passing the Illumina Chastity filtering. Subsequently, reads containing adapters and/or Phix control signal were removed using BaseClear's in-house filtering protocol. The second quality assessment was based on the remaining reads using the FASTQC quality control tool version 0.10.0. The quality of the FASTQ sequences was enhanced by trimming off low-quality bases using the “Trim sequences” option of the CLC Genomics Workbench version 8.0.

For genome assembly and scaffolding, the quality-filtered sequence reads were assembled into contig sequences. The analysis was performed using the “De novo assembly” option of the CLC Genomics Workbench version 8.0. Mis-assemblies and nucleotide disagreement between the Illumina data and the contig sequences were corrected with Pilon version 1.11. The contigs were linked and placed into scaffolds or super-contigs. This resulted in an assembly of 7,315,765 bp and 980 scaffolds. The orientation, order, and distance between the contigs was estimated using the insert size between the paired-end and/or mate-pair reads. The analysis was performed using the SSPACE Premium scaffolder version 2.3. The gapped regions within the scaffolds were (partially) closed in an automated manner using GapFiller version 1.10, taking advantage of the insert size between the paired-end and/or mate-pair reads. The obtained draft genomes were subsequently used for phylogenetic analyses and genome annotations. The quality of the Illumina sequencing de novo genome assembly of P08-G05 was evaluated by the checkM software (version 1.07) showing the high completeness of 95.9% with the low contamination of 1.6%.

### PacBio Sequencing and Hybrid De Novo Genome Assembly

Cell mass for PacBio sequencing and direct cloning was produced in 500 ml shake flasks containing 3 g of 3 mm glass bead and 120 ml TSB medium supplemented with 0.5× artificial sea water at 30° C., 200 rpm with 2.5 orbital movement until OD₆₀₀=5.6. The cell mass was harvested by centrifugation, kept on −40° C. until shipping, and shipped to BaseClear BV, The Netherlands, on dry ice. Long read PacBio sequencing was carried out at BaseClear using the PacBio Sequel instrument, and the obtained data were processed and filtered using the SMRT Link software suite with subreads shorter than 50 bp being discarded. This resulted in a number of 622,557 reads with the yield of 2,886,923, 195 bp.

The quality of the Illumina HiSeq reads was improved by trimming off low-quality bases using BBDuk, which is a part of the BBMap suite version 36.77. High-quality reads were assembled into contigs using ABySS version 2.0.2. The long reads were mapped to the draft assembly using BLASR version 1.3.1. Based on these alignments, the contigs were linked together and placed into scaffolds. The orientation, order, and distance between the contigs were estimated using SSPACE-LongRead version 1.0. Using Illumina reads, gapped regions within scaffolds were (partially) closed using GapFiller version 1.10. Finally, assembly errors and the nucleotide disagreements between the Illumina reads and scaffold sequences were corrected using Pilon version 1.21. This resulted in an assembly of 7,840,734 bp with 22 scaffolds.

### Phylogenetic Positioning of Strain P08-G05

The phylogenetic position of isolate P08-G05 was determined as part of a comprehensive phylogenetic analysis performed on the Illumina HiSeq2500 sequenced genomes of 1200 selected strains of the SINTEF/NTNU marine Actinobacteria strain collection, similarly generated to the one of P08-G05 (as described above), and 576 Actinobacteria type strains retrieved from public databases. Analysis was carried out using IQTREE software (IQ-TREE MPI multicore version 1.6.7.1) with 92 house-keeping genes as reference to identify the phylogenetic novelty of the strain P08-G05. P08-G05 strain was placed among other strains in the Actinobacteria strain collection, not with other type strains, indicating that the strain likely represents a new Actinobacteria species.

### Identification of the Biosynthetic Gene Cluster P08-G05_c16

An in-house Python script was used to evaluate the abundance and diversity of different biosynthetic gene cluster (BGC) classes of the 1200 strains from the Actinobacteria strain collection based on a collection of BGC's profile Hidden Markov models (pHMMs). The obtained matrixes containing the counts of pHMM hits from the corresponding strains were used to cluster the strains into different populations with the usage of an implementation in the programming language R of the t-distributed stochastic neighbor embedding (t-SNE) algorithm by AFG. P08-G05 strain was clustered in the cluster 34 (out of 40 t-SNE clusters) along with other strains. The strain was selected together with other strains from different t-SNE clusters for a shortlist of 86 strains for further characterization, including long-read PacBio sequencing.

The novel cluster, which encodes (at least) the (core) metabolic machinery to synthesize the nidaromycin compound was identified through manual curation based on the antiSMASH result of the PacBio sequenced genome of P08-G05. Analysis of resistance genes on the gene cluster was performed using an in-house script. No resistance genes were identified in the cluster P08-G06_c16.

### Example 3

### Cloning and Expression of Gene Cluster P08-G06_c16

### Cloning and Conjugation of Gene Cluster P08-G06_c16

Based on antiSMASH results, gene cluster P08-G06_c16 was hypothesized to code for a moenomycin-like novel compound. Moenomycin has molecular formula C₆₈H₁₀₆N₅O₃₄P and mass 1567.645683 g/mol.

Cloning of cluster P08-G05_c16 in an inducible Bacterial Artificial Chromosome (BAC) vector (pDualP, proprietary to Varigen Biosciences (Madison, WI, USA)) was purchased from Varigen Biosciences based on chromosomal DNA of strain P08-G05. The construct was received from Varigen Bioscience in an E. coli strain suitable for the propagation of large constructs (10Beta). The cluster was transferred to S. coelicolor M1152ΔmatAB, prepared according to Example 1, by tri-parental conjugation by a procedure that is similar to the previously described methods (Jones et al 2013, PLOS ONE 8 (7): e69319.doe: 10.1371). In short: The BGC containing construct together with the driver plasmid pR9406 were transferred to E. coli ET12567 by triparental mating. For this each strain was first cultivated overnight on LB agar without selection. For all three strains, using an inoculation loop, a couple of colonies were scooped and were streaked together in a patch on LB agar containing apramycin, chloramphenicol and ampicillin. As control, each of the strains were also patched individually on the sample selection. Single colonies of ET12567+pR9406+DualP-BGC were growth in 13 ml culture tubes containing 5 ml LB+ampicillin, chloramphenicol and apramycin until an OD of 0.6, after which the culture was pelleted and washed twice with cold LB media. In parallel the S. coelicolor spores were pre-germinated by heat shock for 10 min at 50° C. and incubation at 30° C. for at 2-3h. E. coli and S. coelicolor spores were mixed and plated on soy flour mannitol (SFM) agar plates and incubated between 18 and 24 h at 30° C., before being overlaid with apramycin+nalidixic acid to select for transconjugant Streptomyces colonies. Single colonies were subsequently patched on selective SFM plates and expanded to confluent plates for spore harvest and storages through standard procedures. The new transconjugant strain carrying the nidaromycin gene cluster was given the short name M1152ΔmatAB (P08-G05_C₁₆).

### Cultivations of the Transconjugant M1152ΔmatAB (P08-G05_C₁₆) and Expression of Gene Cluster P08-G05_c16

Well plate cultivations of M1152ΔmatAB (P08-G05_C₁₆) and M1152ΔmatAB (control) were performed as follows: Seed cultures were produced in 250 ml shake flask with 50 ml of 0.5× Trypton Soya Broth (TSB) and 1.5 g of 3 mm glass bead (without antibiotics) for two days at 30° C. and 225 rpm until OD600=5-7. Production in 24 well plates (AXYGP-DW10ML24C) were performed in both 5254SW medium (Králová et al 2021, Frontiers in Microbiology 12:2131) or MG-2.5 medium (Doull and Vining 1990, Applied Microbiology and Biotechnology, 32, 449-454; Martínez-Castro et al. 2013, Applied Microbiology and Biotechnology, 97, 2139-2152) supplemented with 1 g/L NaCl. The wells were filled with 2.5 ml medium and 4×3 mm glass beads and inoculated with 1.3% from the seed culture. The plates were incubated at 30° C. in a New Brunswick incubator at 800 rpm and 85% humidity for six days. The broth was freeze dried and extracted with one broth volume of DMSO for one hour.

Cell free extracts were analyzed by an Agilent LC-DAD-QTOF equipped with a Zorbax Bonus RP 2.1×50 mm, 3.5 μL. 50 mM ammonium acetate [A] and an acetonitrile [B] were used as mobile phases. The gradient was 5% acetonitrile from 0-2 min, then increasing to 95% for the next 25 min. The QTOF was operated in positive and negative ionization mode with capillary voltage: 3.5 kV, Fragmentor voltage: 150 V, Skimmer: 65V, gas temperature 325° C., drying gass: 10 l/min, Nebulizer: 50. Data was processed using the Mass Hunter and Mass Profiler Professional software from Agilent.

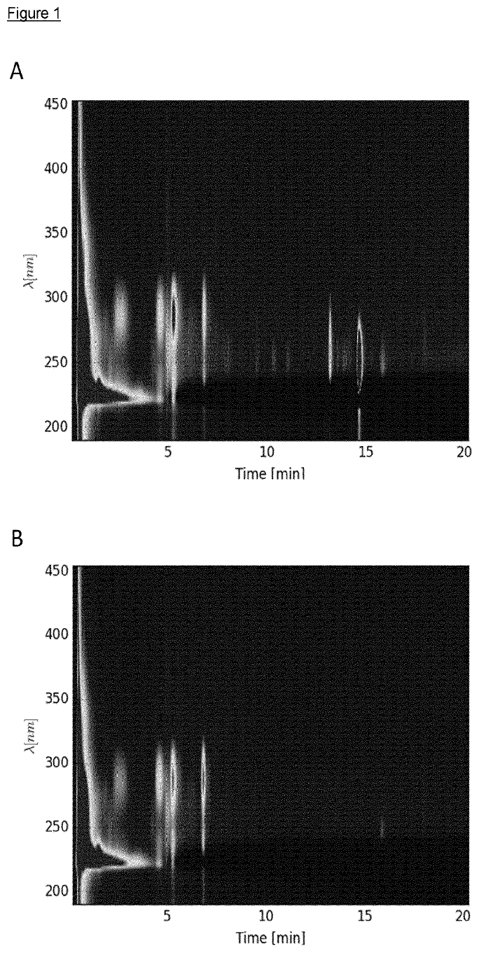

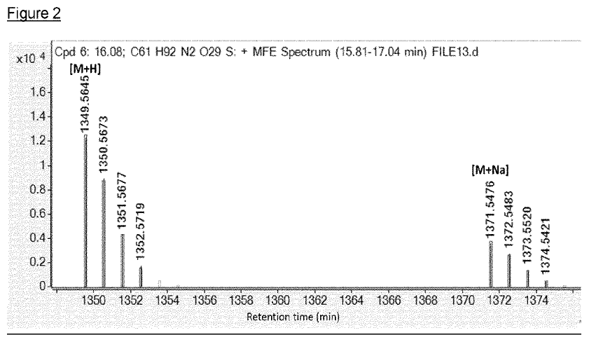

The LC-DAD-isoplots showed that two peaks were observed in the transconjugant extract but not in the control (FIG. 1). The abundance of these two peaks were higher in the MG-2.5 w/0.5× seawater than in the 5254SW medium. The MS data showed that a cluster of masses was observed at the retention time that corresponded to the main UV peak. The three dominating masses were M+H=1349.5668 and it's adduct M+Na=1371.5496 (FIG. 2). These masses were not found in extracts of the control M1152ΔmatAB. The compound was concluded to be associated with the heterologous expression of the introduced P08-G05_c16 and named nidaromycin.

### Example 4

### Up-Scaled Production and Purification of the Heterologous Expressed Compound

Up-scaled production of active compound was performed in 500 ml shake flasks with 125 ml of MG-2.5 w/NaCl. The medium was inoculated with 3% from seed culture and incubated at 30° C. for six days at 200 rpm with 2.5 cm orbital movement.

The broth was freeze dried and homogenized with mortar. The material was extracted with DMSO acidified with trifluoroacetic acid (TFA) to 0.1% final concentration. The amount of organic solvent was 0.4× original broth volume. The DMSO extract was fractionated using an Agilent preparative HPLC equipped with a Zorbax Bonus RP, 9.4×250 mm, 7 μm column (Agilent), diode array detector (DAD) and a fraction collector. Mobile phases were water with 20 mM ammonium acetate [A] and acetonitrile [B]. The gradient was 5% [B] during injection, then a gradient increase from 55% to 75% [B] over 10 min. The column was washed for 1 min with 95% [B] before column equilibration with 5% [B]. The acetonitrile in the HPLC fractions was removed by rotational evaporator, and the aqueous phase was further purified and concentrated using 500 mg HLB solid phase extraction columns (Waters). The compound was eluted from the SPE column with methanol. The methanol was removed by evaporation using a Speedvac (ThermoFisher) at 50° C. The sample was added water, frozen at −80° C. and freeze dried.

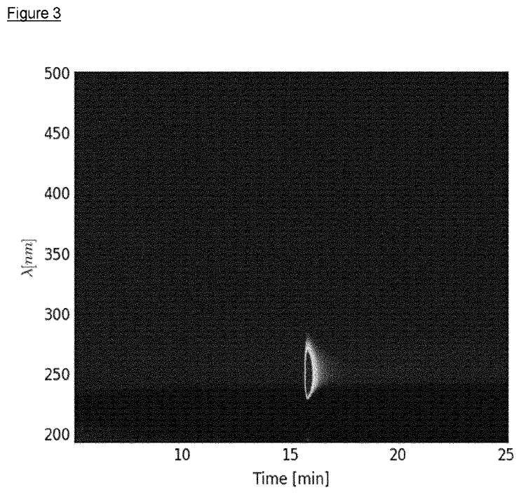

The DAD plot in FIG. 3 confirms that a purified compound was obtained. The material obtained was used for inhibition and toxicity assays as described in Example 5, determining the molecular formula of nidaromycin as described in Example 6, as well as structure elucidation by NMR and determining the position of the sulphate group as described in Examples 7 and 8.

### Example 5

### Assay of Activity of Crude Extract and Purified Compound

### Bioassay of Crude Extract

From cultures of strain M1152ΔmatAB (P08-G05_C₁₆) (prepared as described in Example 3), cell free extract was prepared and tested in bioassay against a panel of strains, i.e., Enterococcus faecium CCUG 37832, M. luteus TO-09 ATCC9341, Pseudomonas aeruginosa ATCC 15692 and C. albicans CCUG. The extract showed activity against E. faecium CCUG37832.

In particular, extracts of the transconjugant inhibited growth of E. faecium CCUG 37832 at 16× dilution (MG-2.5 w/NaCl) and 4× dilution (5254SW), whereas extracts of the control did not inhibit any of the strains.

### In Vitro MIC Bioassay

Minimal inhibitory concentrations against a selection of Gram-positive indicator organisms (MICs) were determined according to Clinical and Laboratory Standards Institute protocols by microdilution tests using in a 384 well format. The indicator strains were incubated in TSB medium over night until OD600-0.4, then diluted to OD600=0.1 in TSB medium and further 45× in assay medium (Mueller-Hinton broth, Difco). Inoculated medium was distributed into assay plates. Inoculated wells were added a 2× dilution series of either isolated compound or vancomycin (reference) diluted in DMSO, giving a final DMSO concentration in each well of 2.7%, and final concentrations of active compound between 0 and 540 μg/ml (23 different concentrations). Four parallels were assayed for each compound and concentration. 0.5 mg of the compound produced by strain M1152ΔmatAB (P08-G05_C₁₆) was purified on preparative HPLC, and the pure compound was tested in bioassay against a panel of strains. The indicator organisms were M. luteus ATCC 9341, Staphylococcus aureus ATCC 29213, Staphylococcus aureus ATCC 43300 (MRSA), Enterococcus faecium CCUG 37832, Enterococcus faecium CTC 492. The results are shown in Table 3 below.

TABLE 3

MIC70 (μg/ml)

| | Nidaromycin | Vancomycin |
|---|---|---|
| MIC₇₀ S. aureus ATCC 29213: | 0.53 | 1.06 |
| MIC₇₀ S. aureus ATCC 43300 | 0.53 | 1.06 |
| MIC₇₀: E. faecium CTC 492 | 8.45 | 1.06 |
| MIC₇₀: E. faecium CCUG 37832 | 2.11 | 270 |

### In Vitro Toxicity Assay

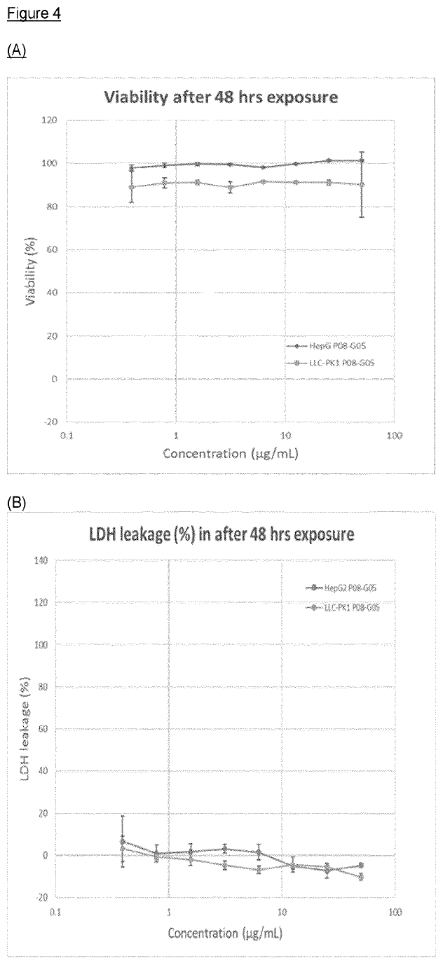

Toxicity of nidaromycin against human cell lines was evaluated with the human cell lines HepG2, LLC-PK1 and L929 cultivated in RPMI 1640 supplemented with 10% Fetal Bovine Serum (FBS), 2 mM L-glutamine and 100 U/ml Pen-Strep (HepG2), Medium 199 supplemented with 3% FBS, 2 mM L-Glutamine, 100 U/ml Pen-Strep (LLC-PK1) and Dulbecco's Modified Eagle Medium (DMEM) supplemented with 10% FBS, 2 mM L-glutamine, 1 mM Sodium pyruvate and 100 U/ml Pen-Strep (L929). The cells were sub-cultured according to standard protocols, and the cell suspensions was transferred from a stirred reservoir and seeded into 384-well plates (Corning Assay Plate, 3712) using Tecan EVO robotic workstation with MCA384 pipetting unit using disposable tips (Tecan MCA 125 ui, Cat No. 300-5-1-808). The reservoir (flat base, 300 mL, Thermo Scientific, 10723363) was equipped with sterile magnetic stirring bars (15×4.5 mm VWR 442-4522) stirring at 350 rpm. The number of cells in each well was 50.000 (HepG2), 25.000 (LLC-PK1) and 10.000 (L929). The microplates with cell suspension were shaken at 1600 rpm with 2.5 mm amplitude (Bioshake) for 20 seconds after seeding. The microplates with the cells were incubated at 37° C. with 5% CO2 atmosphere. At the day of the exposure of the cells, serial dilutions were made in DMSO. The serial dilutions with the compounds were further diluted in cell culture medium and transferred to the assay wells, giving a total DMSO concentration in the assay wells of 0.6%. After exposure, the plate was further incubated at 37° C. with 5% CO2 atmosphere for 48 hours. The viability of the cells after incubation for 24 hours and 48 hours was measured using the Promega CellTiter-GLO 2.0 viability assay. The highest concentration tested was 50 g/ml of Nidaromycin, and no toxic effect was detected for this concentration or lower. The data are shown in FIG. 4.

### Example 6

### Determining the Molecular Formula of Nidaromycin

### MS1 Analyses of Isotope Labelled Broth

To determine the molecular formula of nidaromycin, strain M1152ΔmatAB (P08-G05_C₁₆) was cultivated in isotope labelled medium where all carbon sources were ¹³C labelled and all nitrogen sources were ¹⁵N labelled as follows: 3 ml of seed culture, produced in 0.5× TSB medium, was washed once with ml of sterile 0.9% NaCl, resuspended in 0.9% NaCl to OD=5 and used to inoculate production cultures (0.5% in 20 ml medium). Productions were performed in 250 ml shake flasks with 1.5 g of 3 ml glass beads and 20 ml of the following media from Silante: 1 g/100 ml ¹³C Silex Media Powder for E. coli (115204100), E. coli OD2N (110301402) or E. coli OD2CN (110601402). The culture was harvested after six days.

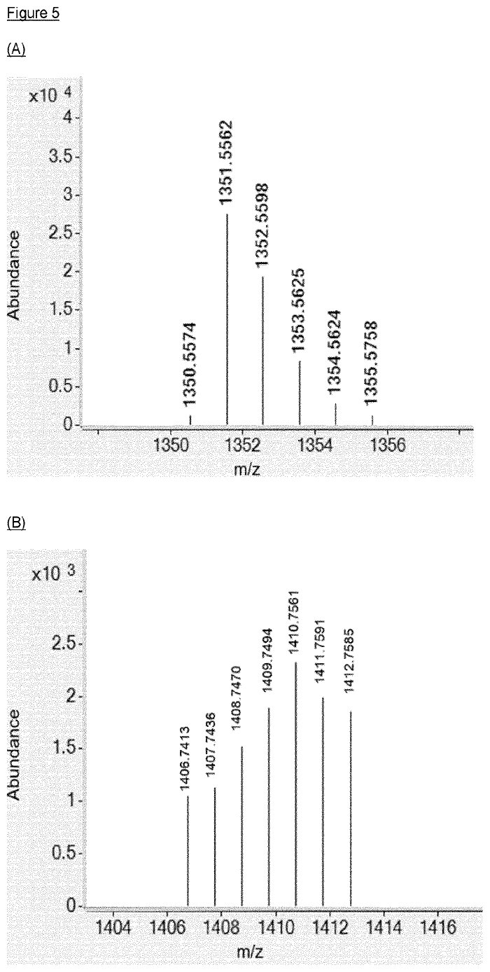

Freeze dried isotope labelled broths were extracted one hour with 2 ml DMSO added trifluoroacetic acid to 0.1% per 20 ml original broth volume The volumetric yield of nidaromycin in these media was very low, but high enough to detect the masses of isotope labelled nidaromycin. The masses of ¹³C, ¹⁵N, and ¹³C and ¹⁵N labelled nidaromycin were 1410.7769 Da, 1351.5562 Da and 1412.7585 Da, respectively, showing that the molecular formula of nidaromycin contains 61 carbons and 2 nitrogens (FIG. 5). Based on MS1 data (FIG. 2), the most likely molecular formula was C₆₁H₉₂N₂O₂₉S giving a theoretical monoisotopic mass of 1348.5507 Da.

### MS2 Analysis of Purified Nidaromycin

Purified nidaromycin was analysed with LC-MSMS with fragmentation of the precursor mass m/z=1349.567. The LC-conditions were the same as described in Example 4, and the MSMS data was generated using a Bruker Impact II QTOF in positive mode. The MS conditions were: Spectra rate: 12 Hz, Capillary voltage: 4500 V, Endplate offset: 500V, drying gas: 10 L/min, Nebulizer gas: 220, Data acquisition control: dynamic MSMS, collision energy 5V with multiCE 20, 50 and 100.

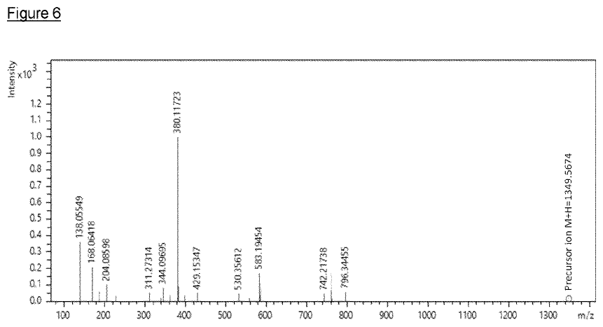

The molecular mass and the MSMS fragmentation pattern (FIG. 6) suggested that the molecular formula was C₆₁H₉₂N₂O₂₉S and 29 of the MSMS fragments could be explained by this formula (Data not shown).

### Example 7

### Structure Determination by NMR

The compound produced by the transconjugant M1152ΔmatAB (P08-G05_C16) was subjected to structure determination by 1D and 2D NMR spectroscopy by Red Glead Discovery AB.

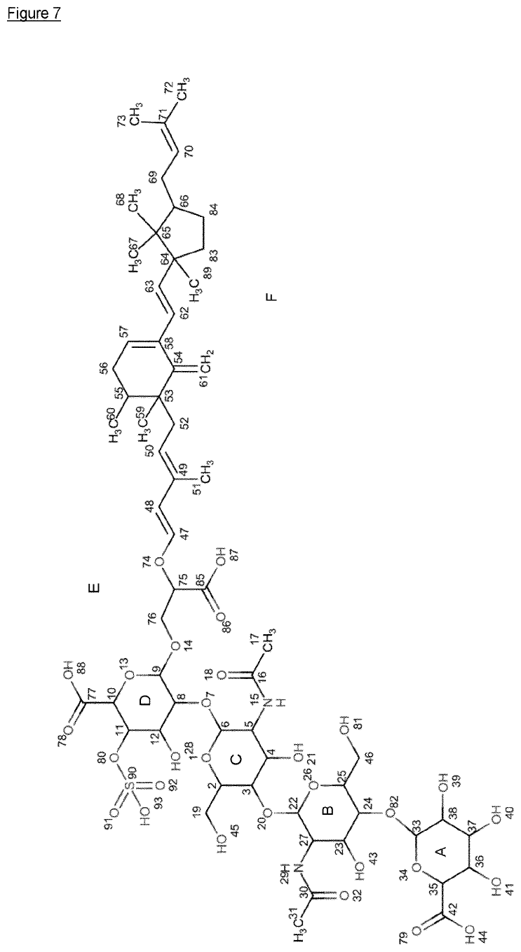

The determined structure is shown in FIG. 7, which also shows the atom numbering for the atom-specific assignments which have been made. The structure consists of four substituted sugar moieties A-D, a linking 2,3-dihydroxypropionic acid (E) and a hydrocarbon moiety with the formula C30H45 (F). There are several alternative positions for the proposed sulphate group, which is shown at position 4 on the uronic acid unit “D”, that may be valid. The alternative positions are position 3 in unit D and position 4 in unit A, as shown below:

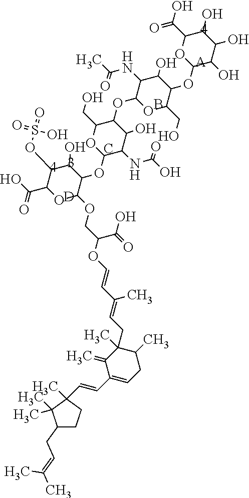

The NMR studies made are detailed below.

### Sample Information

The studied sample was provided to ReadGlead as solid material. The material was stored at −20° C. upon reception. Prepared NMR samples were stored dark at 4-8° C. in between measurements. The following sample information was provided:
Sample ID: P08-G05_c16
Monoisotopic mass: 1348.5604
Formula sum: C61H92N2O29S
Obtained Amount: 5.13 mg
Solubility: 10 mg/mL in DMSO upon ultrasonication

### Material & Methods

NMR samples

### PN102-62-01

The NMR sample was prepared by weighing up 2.899 mg of sample P08-G05_c16 in a screw cap vial and adding 540 μL DMSO-d₆. The sample dissolved slowly and was heated to 40° C. for 1-2 minutes and before being ultrasonicated for 3×10 seconds. The sample still showed traces of finely dispersed undissolved particles, as controlled by visual inspection, but was transferred to a 5 mm NMR tube.

### PN102-62-01B

The NMR sample was prepared by adding 20 μL D₂O to the NMR tube of sample PN102-62-01 above.

### PN102-62-01C

The NMR sample was prepared by adding 2 μL TFA-d to the NMR tube of sample PN102-62-01B above.

### PN102-62-02

The NMR sample was prepared by adding 540 μL CD3OD directly to the Eppendorf tube containing the remains of P08-G05_c16 (approx. 2.2 mg). The sample dissolved slowly and was heated to 40° C. for 1-2 minutes and before being ultrasonicated for 3×10 seconds. The sample still contained significant amounts of undissolved material, as controlled by visual inspection, but the supernatant was transferred to a 5 mm NMR tube.

### Chemicals and Materials

### Equipment:

Mettler Toledo MT5 balance
Bandelin Sonorex ultrasonic bath, model no. RK 31
Agilent 2 mL clear screw neck vials, Part No. 5190-9062
Agilent Technologies screw caps, 9 mm with PTFE/Silicone septa, Part No. 5190-9068
Hilgenberg Standard NMR tubes, 5 mm diameter, Item No. 2001745
Hilgenberg closing caps for NMR tubes, 5 mm diameter, Item No. 9400312

NMR solvents and chemicals

| Name | CAS# | Lot# | Supplier | Cat# |
|---|---|---|---|---|
| DMSO-d₆ (99.9 | 2206- | 11578 | ARMAR | 015200.0009 |
| atom % D) | 27-1 | | Chemicals | |
| D₂O (99.9 | 7789- | B 19908 | Deutero | 00506-25ml |
| atom % D) | 20-0 | | | |
| CD₃OD (99.8 | 811- | B 15576 | Deutero | 01105-25ml |
| atom % D) | 98-3 | | | |
| TFA-d (99.5 | 599- | MKCH3593 | Aldrich | 152005- |
| atom % D) | 00-8 | | | 10X0.5ML |

### NMR Spectroscopy

A 500 MHz Bruker Avance Neo spectrometer equipped with a 5 mm iProbe BBF/H/D probe and a 500 MHz Varian Inova spectrometer equipped with a 5 mm 1H/¹³C/¹⁵N triple resonance probe were used for the performed NMR experiments. Data were recorded at 25° C. or 40° C. The recorded spectra are listed in Table 4 below.

TABLE 4

| NMR experiment | PN102-62-01 | PN102-62-01B | PN102-62-01C | PN102-62-02 |
|---|---|---|---|---|
| 1D ¹H | X | X | X | X |
| (standard sequence “zg30” in | | | | |
| TopSpin 4.0.8 with a 30° pulse | | | | |
| or standard sequence “s2pul” | | | | |
| in VnmrJ 2.3, 45° pulse; or | | | | |
| quantitative measurements | | | | |
| with 90° pulse and d1 = 57 s) | | | | |
| 1D ¹³C | X | | X | |
| (standard sequence “zgpg30” | | | | |
| in TopSpin 4.0.8) | | | | |
| 1D ³¹P | X | | X | |
| (standard sequence “zgpg30” | | | | |
| in TopSpin 4.0.8) | | | | |
| 2D ¹H COSY | X | X | X | X |
| (standard version “cosygpppqf” | | | | |
| in TopSpin 4.0.8 or standard | | | | |
| version gCOSY in VnmrJ 2.3) | | | | |
| 2D ¹H TOCSY | X | X | X | X |
| (standard version “mlevphpp” | | | | |
| in TopSpin 4.0.8 or standard | | | | |
| version “zTOCSY” in VnmrJ | | | | |
| 2.3, mixing time set to 60 or 80 | | | | |
| ms) | | | | |
| 2D ¹H NOESY | X | X | X | X |
| (standard version | | | | |
| “noesygpphpp” in TopSpin | | | | |
| 4.0.8 or standard version | | | | |
| “NOESY” in VnmrJ 2.3, mixing | | | | |
| time set to 200 or 300 ms) | | | | |
| 2D ¹H ROESY | X | | | |
| (standard version | | | | |
| “roesyphpp.2” in TopSpin | | | | |
| 4.0.8, mixing time set to 200 | | | | |
| ms) | | | | |
| 2D ¹H-¹³C HSQC | X | X | X | X |
| (multiplicity edited: standard | | | | |
| version “hsqcedetgpsisp2.3” in | | | | |
| TopSpin 4.0.8, or in VnmrJ 2.3: | | | | |
| gradient version kindly | | | | |
| provided by Lewis Kay's NMR | | | | |
| laboratory, University of | | | | |
| Toronto, Canada; implemented | | | | |
| by Yamazaki 1993) | | | | |
| 2D ¹H-¹³C HMBC | X | X | X | X |
| (standard version | | | | |
| “hmbcetgpl3nd” in TopSpin | | | | |
| 4.0.8 or standard version | | | | |
| “gHMBC” in VnmrJ 2.3, | | | | |
| optimized for J = 5, 8 or 10 Hz) | | | | |
| 2D ¹H-¹⁵N HSQC | X | | | |
| (standard version | | | | |
| “hsqcetgpsi2” in TopSpin 4.0.8) | | | | |
| 2D ¹H-¹⁵N HMBC | X | | | |
| (standard version | | | | |
| “hmbcgpndqf” in TopSpin | | | | |
| 4.0.8) | | | | |

The solvent residual signals of DMSO-d₆ (2.50/39.52 ppm) and CD₃OD (3.31/49.00 ppm) were used as the reference for ¹H and ¹³C chemical shifts.

NMR data were processed and analysed using MestreNova 12.0.1 (Mestrelab Research S. L.). Chemical shift predictions were performed with the plug-in “NMRPredict” in MestReNova 12.0.1, using the predictor “Mnova Best” with solvent set to DMSO-d₆.

### Results and Conclusions

### NMR Experiments and Conditions

The NMR experiments were performed on the obtained material dissolved in DMSO-d₆ or CD₃OD. NMR data have been recorded on a 500 MHz Bruker Avance NMR spectrometer and a 500 MHz Varian Inova spectrometer.

1D and 2D ¹H/¹³C/¹⁵N NMR spectral data of moderate quality were acquired for the provided sample material in DMSO-de. A significant number of low-intensity signals were observed, possibly referring to structurally related impurities and/or minor conformers of the main species in solution. The spectral region of the sugar moieties was complicated by signal overlapping and signal broadening, to the extent that ¹H-¹³C HSQC cross-peaks in a few cases were not readily observable. Heating the DMSO-de sample to 40° C. resulted in no or only very minute sharpening of the signals and thus all data used in the structure elucidation were recorded at 25° C. Acidification of the DMSO-de sample with TFA-d, however, resulted in significant sharpening of some signals, and also chemical shift changes of the sugars and sugar derivatives (while the chemical shifts of the hydrocarbon tail remained essentially unaffected).

The overall appearance of the CD₃OD spectra were slightly clearer and sharper than the DMSO-d₆ spectra. However, due to limitations in solubility, the signal-to-noise ratio intensity in methanol was too poor to achieve useful 2D long-range and through-space NMR data, critical for the structure identification process. The CD₃OD data set could still provide useful information in a few cases where DMSO-de data were not unambiguous.

In all, four different NMR data sets were used for the structure elucidation. For completeness, the chemical shift assignment of the suggested structure is reported for both the DMSO-de sample (PN102-62-01) and the DMSO-de/TFA-d sample (PN102-62-01C), except for the hydrocarbon tail “F” where chemical shifts are very alike in the two samples. Besides the main compound, the sample also appears to contain significant amount of an unassigned small molecule.

### Elucidation of Chemical Structure and Atom Specific Assignment

The suggested structure of P08-G05_c16 (FIG. 7) shares several structural features with related moenomycin compounds in that they all contain a substituted tetrasaccharide linked to a hydrocarbon tail. However, as opposed to the moenomycins, P08-G05_c16 lacks the linking phosphodiester and the hydrocarbon tail contains 30 instead of 25 carbon atoms. Structural evidence is strong for P08-G05_c16 as essentially all 1H/¹³C/¹⁵N atoms are observed and assigned, except for the amide nitrogen of sugar unit “C” and the carbonyl carbon of the 2,3-dihydroxypropionic acid unit “E”, and the atom connectivity is fully consistent with the obtained 2D data. Further, a quite good degree of agreement is observed between the experimental and predicted chemical shift values.

The sugar units denoted “A” and “D” are assigned as uronic acids and “B” and “C” as N-acetyl-glucosamines. Stereospecific assignments are not included in the present study. The connectivity of the sugar moieties was determined by the correlations between the anomeric proton signals and the corresponding carbon signals (C-4 of sugars “B” and “C” and C-2 of sugar “D”) through glycoside bonds in the HMBC spectra and/or NOE correlations between the anomeric proton signal and the corresponding proton in the next sugar moiety. The connectivity of sugar “D” and the linking 2,3-dihydroxypropionic acid “E” is confirmed by NOE correlations between the anomeric proton of “D” and the methylene protons of “E” (only observed for the acidified sample PN102-62-01C). The connectivity between “E” and the hydrocarbon tail “F” is also established through observed NOEs, between both the CH and CH2 protons of “E” and the two closest CH protons of “F” (atom no. 47 and 48 in Table 6).

The assigned chemical shifts for P08-G05_c16 are presented in Tables 5 and 6 below, along with the corresponding chemical shifts predicted from the proposed chemical structure. The overall picture is that predicted chemical shifts values fully support the suggested molecular structure, and that minor deviations from the predicted values are only observed for the “D” moiety.

Based on the suggested formula sum, a sulphate substituent on one of the sugar oxygens has been postulated. There are several available positions for this, i.e. sugar positions where the OH proton is not detected, and no other substituent/linkage is determined. As the ¹³C chemical shifts for C-3 and C-6 are very similar in moiety “B” as compared to moiety “C”, it is deemed unlikely that any of those positions should bear a sulphate. Thus, the positions C-4 of unit “A”, C-3 of unit “D” and C-4 of unit “D” are left as plausible candidates. The position of the sulphate cannot be defined solely based on the NMR data. However, as ring system “D” is more sensitive to changes in pH and also appears to have a higher structural complexity than system “A” due to deviations from predicted chemical shift values, it is considered the more plausible option. O-sulphation can be expected to cause slightly downfield shifts for both the O-sulphated carbon and the proton bound to it, and for this reason position C-4 of unit “D” has been tentatively assigned.

### Table 5

Chemical shift (o) values for P08-G05_c16, part A-D in FIG. 8, predicted with “Mnova Predict” and experimentally determined in DMSO-d₆ (NMR sample PN102-62-01) and DMSO-d₆ after addition of D₂O and TFA-d (NMR sample PN102-62-01C). Experimental shifts are reported relative the solvent residual signal for 1H/¹³C (2.50/39.52 ppm) while indirect referencing is applied for ¹⁵N. Data are recorded at ° C. NO=not observed.

| Atom | Predicted δ | DMSO-d₆, δ | DMSO-d₆/TFA, δ |
|---|---|---|---|
| no. | ¹H/¹³C [ppm] | ¹H/¹³C [ppm] | ¹H/¹³C [ppm] |
| 2 | 3.84/75.6 | 3.18/75.1 | 3.22/75.0 |
| 3 | 3.94/79.0 | 3.27/81.3 | 3.22/81.5 |
| 4 | 4.11/72.3 | 3.52/72.2 | 3.50/72.2 |
| 5 | 3.74/55.4 | 3.45/55.1 | 3.50/54.7 |
| 6 | 5.27/101.3 | 4.51/101.7 | 4.53/101.8 |
| 8 | 4.02/79.3 | 3.75/NO | 3.84/72.8 |
| 9 | 5.04/101.9 | 5.67/NO | 5.77/95.4 |
| 10 | 3.97/75.9 | 4.39/NO | 4.58/67.1 |
| 11 | 5.22/75.8 | 3.86/NO | 3.92/70.5 |
| 12 | 4.09/73.1 | 3.71/NO | 3.81/68.7 |
| 15 | 7.57/87.8 (¹⁵N) | 7.94/NO | NO/NO |
| 16 | 172.3 | 169.7 | 169.3 |
| 17 | 2.02/23.2 | 1.82/23.0 | 1.82/22.8 |
| 19 | 3.77, 3.91/61.6 | 3.43, 3.59/60.0 | 3.43, 3.60/60.2 |
| 21 | 4.19 | 4.67 | NO |
| 22 | 5.24/101.6 | 4.37/101.9 | 4.36/101.8 |
| 23 | 4.11/72.4 | 3.49/72.3 | 3.46/71.8 |
| 24 | 3.98/79.8 | 3.30/80.3 | 3.35/79.9 |
| 25 | 3.86/75.9 | 3.31/75.2 | 3.35/74.9 |
| 27 | 3.75/55.2 | 3.51/54.9 | 3.48/54.8 |
| 28 | 4.57 | NO | NO |
| 29 | 7.57/87.9 (¹⁵N) | 7.88/118.4 (¹⁵N) | NO/NO |
| 30 | 172.4 | 168.9 | 169.8 |
| 31 | 2.02/23.3 | 1.82/23.0 | 1.81/22.9 |
| 33 | 5.18/103.2 | 4.23/102.8 | 4.37/102.9 |
| 35 | 3.77/76.0 | 3.33/73.4 | 3.72/75.0 |
| 36 | 3.61/72.6 | 3.12/72.0 | 3.28/71.2 |
| 37 | 3.85/76.0 | 3.14/76.4 | 3.18/75.7 |
| 38 | 3.81/74.6 | 2.99/73.1 | 3.03/72.8 |
| 39 | 4.40 | 5.17 | NO |
| 40 | 4.72 | 4.98 | NO |
| 41 | 5.20 | NO | NO |
| 42 | 173.4 | NO | 170.0 |
| 43 | 4.13 | NO | NO |
| 46 | 3.82, 3.89/61.5 | 3.53, 3.81/60.2 | 3.52, 3.79/59.8 |
| 77 | 172.1 | NO | 170.3 |

Amide nitrogen no. 15 in sugar “C” is not observed from the recorded 1H-¹⁵N HSQC data. This is an expected result due to the observed broadening of the associated amide proton signal in the 1D 1H spectral data. Nonetheless, the structural assignment of the acetamido sugar “C” is very likely, due to the characteristic chemical shifts of both the amide proton and the adjacent C-2 carbon, along the with the overall conformity between moieties “B” and “C” and the anticipated formula sum. Similarly, the carbonyl carbon no. 85 in moiety “E” is not observed from the recorded ¹H-¹³C HMBC data, as would be expected due to the broadening observed for the adjacent methine no. 75. The suggested substructure of “E” is still probable, due to the close agreement between predicted and experimental ¹³C chemical shift values for methine no. 75, the known presence of this structural motif in related moenomycins and the overall formula sum. The high number of quaternary carbon atoms and the splitting of the methylene proton signals observed for fragment “F” accounts for the cyclic subunits, that also agrees with the total number of cycles and double bonds expected for the suggested formula sum. The structural unit “F” significantly deviates from the moenomycin structure(s) and has not been evaluated from a biosynthetic point of view.

The structural moieties “D” and “E” display significantly broadened 1H signals in DMSO-d₆, with crosspeaks in ¹H-¹³C HSQC data broadened beyond recognition. The signals are sharpened upon addition of TFA-d to the sample, allowing for ¹³C chemical shift assignments, but data then also reveals a tendency for doubling of these signals—the sharpening effect in this region are consistent with the presence of the carboxylic acid moieties being protonated upon acidification. The origin behind these observations have not been explored within the present study and only the major signals have been evaluated in the structure elucidation.

### Table 6

Chemical shift (δ) values for P08-G05_c16, part E-F in FIG. 8, predicted with “Mnova Predict” and experimentally determined in DMSO-de (NMR sample PN102-62-01). * Shifts reported in DMSO-de after addition of D₂₀ and TFA-d (NMR sample PN102-62-01C). Experimental shifts are reported relative the solvent residual signal for 1H/¹³C (2.50/39.52 ppm) while indirect referencing is applied for ¹⁵N. Data are recorded at 25° C. NO=not observed.

| Atom | Predicted δ | DMSO-d₆, δ |
|---|---|---|
| no. | ¹H/¹³C [ppm] | ¹H/¹³C [ppm] |
| 47 | 6.58/147.7 | 6.51/148.6 |
| 48 | 5.19/118.4 | 5.82/105.3 |
| 49 | 135.4 | 130.2 |
| 50 | 5.44/127.7 | 4.95/121.5 |
| 51 | 1.80/14.2 | 1.68/20.8 |
| 52 | 2.14, 2.20/36.0 | 2.13, 2.21/35.3 |
| 53 | 43.0 | 41.0 |
| 54 | 150.2 | 149.1 |
| 55 | 1.81/37.4 | 1.71/34.5 |
| 56 | 2.08, 2.12/33.0 | 1.84, 2.42/31.2 |
| 57 | 5.63/129.1 | 5.66/123.5 |
| 58 | 139.5 | 135.8 |
| 59 | 0.95/20.3 | 0.93/21.9 |
| 60 | 0.91/15.9 | 0.74/16.0 |
| 61 | 5.30/113.5 | 4.89, 5.03/109.7 |
| 62 | 6.23/127.5 | 5.89/125.7 |
| 63 | 5.80/139.1 | 5.78/138.7 |
| 64 | 50.4 | 49.4 |
| 65 | 43.6 | 45.3 |
| 66 | 2.07/45.9 | 1.70/47.7 |
| 67 | 0.87/22.3 | 0.70/18.5 |
| 68 | 0.92/22.3 | 0.77/22.5 |
| 69 | 1.75, 2.25/28.5 | 1.74, 2.01/29.2 |
| 70 | 5.02/124.8 | 5.09/124.3 |
| 71 | 133.3 | 130.3 |
| 72 | 1.62/23.4 | 1.64/25.6 |
| 73 | 1.62/20.2 | 1.56/17.7 |
| 75 | 4.55/74.2 | 4.38/NO |
| 75* | 4.55/74.2 | 4.68/77.4* |
| 76 | 3.97, 3.99/66.2 | 3.94, 4.04/NO |
| 76* | 3.97, 3.99/66.2 | 4.16, 4.27/65.8* |
| 85 | 172.0 | NO |

### Example 8

### Determining the Position of the Sulphate Group in Nidaromycin

### BACKGROUND

ReadGlead has determined the structure of Nidaromycin (the active compound produced by P08-G05_c16). However, there were some uncertainties regarding where the sulphate group (SO4-group) is. Here we used MSMS fragmentation followed by in silico fragmentation with the aim of determine the position of the SO4-group in Nidaromycin.

Based on the suggested formula sum, a sulphate substituent on one of the sugar oxygens has been postulated. There are several available positions for this, i.e. sugar positions where the OH proton is not detected, and no other substituent/linkage is determined. As the ¹³C chemical shifts for C-3 and C-6 are very similar in moiety “B” as compared to moiety “C”, it is deemed unlikely that any of those positions should bear a sulphate. Thus, the positions C-4 of unit “A”, C-3 of unit “D” and C-4 of unit “D” are left as plausible candidates. The position of the sulphate cannot be defined solely based on the NMR data. However, as ring system “D” is more sensitive to changes in pH and also appears to have a higher structural complexity than system “A” due to deviations from predicted chemical shift values, it is considered the more plausible option. O-sulphation can be expected to cause slightly downfield shifts for both the O-sulphated carbon and the proton bound to it, and for this reason position C-4 of unit “D” has been tentatively assigned.

The three possible structures are given with SMILES:

Nidaromycin A4
C/C(C)═C\CC1CCC(C)(/C═C/C2═CCC(C)C(C)(C\C═C(\C)/
C═C/OC(COC3OC(C(═O)O)C(O)C(O)C3OC3OC(CO)C(OC4OC
(CO)C(OC5OC(C(═O)O)C(OS(═O)(═O)O)C(O)C5O)C(O)C4/
N═C(\C)O)C(O)C3\N═C(/C)O)C(═O)O)C2═C)C1(C)C
Nidaromycin D3.
CC(CC═C1/C═C/C2(C)C(C)(C)C(CC═C(C)C)CC2)C(C)(C/
C═C(\C)/C═C/OC(COC(C(C(C2O)OS(O)(═O)═O)OC(C(C3O)
NC(C)═O)OC(CO)C3OC(C(C3O)NC(C)═O)OC(CO)C3OC(C(C
(C3O)O)O)OC3C(O)═O)OC2C(O)═O)C(O)═O)C1═C
Nidaromycin D4:
CC1CC═C(\C═C\C2(C)CCC(CC═C(C)C)C2(C)C)C(═C)C1
(C)C\C═C(/C)\C═C\OC(COC1OC(C(OS(O)(═O)═O)C(O)
C1OC1OC(CO)C(OC2OC(CO)C(OC3OC(C(O)C(O)C3O)
C(O)═O)C(O)C2NC(C)═O)C(O)C1NC(C)═O)C(O)═O)C(O)═O

### Materials and Methods:

LC-MS-method: Cell free extracts were analyzed using an Agilent LC-DAD system connected to a Bruker Impact II QTOF. The LC was run with 10 mM ammonium acetate buffer [mobile phase A] and 90:10 acetonitrile:water with 10 mM ammonium acetate [mobile phase B]. The gradient was 5% B for 2 min, then 5-100% B for 2-25 min. The MS was performed at electrospray ionization at positive mode with the following MS parameters: Mass range 100-1800, Spectra rate: 12 Hz, Absolute threshold: 25 counts, threshold for fragmentation: 100 counts, capillary voltage: 4500V, endplate offset: 500V, drying gas: 10 L/min, Nebulizer: 31.9 psi, Drying temperature: 220° C., Precursor ion list: 1000-1500, Data acquisition control: Dynamic MSMS or Fixed MSMS, collision energy: 5V, CID: acqCtr+MultiCe, MultiCe20. In silico fragmentation. In silico fragmentation was performed using MetFrag (Schymanski et al., 2015 Anal Bioanal Chem 407 (21): 6237-6255).

### Results:

Investigating the fragments: The in silico fragmentation strongly suggest that the SO4-group is positioned at either D3 or D4 and not A4. As shown in Table 7, there are several fragments that should not be formed if the SO4-group is positioned in A4. Further, it is difficult to distinguish between D3 and D4 since these structures in general will give the same fragments. In addition, the SO4-group is often lost in the fragmentation, and only a few low abundant fragments contain the SO4-group. However, we observe one fragment (M+H=398.1976) that can be explained by D3, but not by D4. This is an indication that the SO4-group is positioned in D3, but this is supported by only one matching fragment.

To support the QTOF-data, data from FT-ICR MSMS fragmentation at negative ionization was inspected. Not even the FT-ICR fragmentation pattern could distinguish between the position D3 and D4. However, several fragments could only be explained by sulphate in either position D3 or D4 and not by position A4 (Table 5).

### Table 7

Fragments obtained with MSMS fragmentation using Bruker Impact II QTOF were compared with in silico fragmentation of the three suggested structures. Several of the fragments could not be explained by the structure with the SO4-group in the A4 position.

| Mass and formula | A4 | D3 | D4 |
|---|---|---|---|
| 380.1185Da [C14H22NO11]+ | 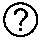 | 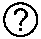 | 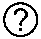 |
| 398.198Da [C20H28O8+H]+H+ | ND | 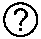 | ND |
| 429.1454Da [C15H24NO13+2H]+H+ | ND | 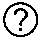 | 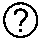 |
| 759.2290 [C28H42N2O22]+H+ | ND | 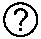 | 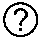 |

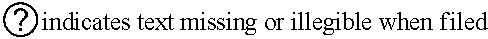

### Table 8

Fragments obtained with MSMS fragmentation using Bruker FT-ICR were compared with in silico fragmentation of the three suggested structures. The fragments shown here could not be explained by the structure with the SO4-group in the A4 position.

| Mass and formula | A4 | D3 | D4 |
|---|---|---|---|
| [C19H25O12S]−H− 476.09936 | ND | 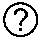 | 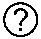 |
| [C40H57NO14S+2H]− 809.3678 | ND | 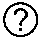 | 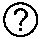 |
| [C42H61NO18S+2H]− 901.37914 | ND | 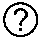 | 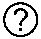 |

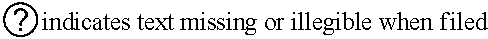

1. A compound of formula (I): 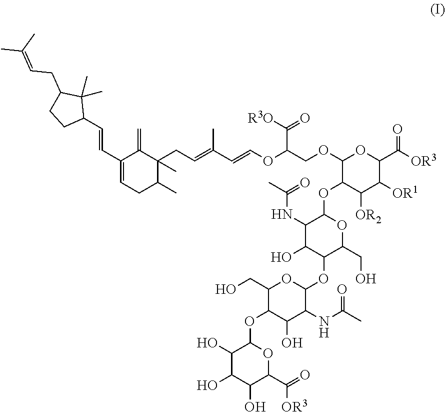
wherein R¹ is —SO₂OH, —SO₂OR or —SO₂R and R² is H, or wherein R² is —SO₂OH, —SO₂OR or —SO₂R and R¹ is H;
wherein R is a C₁-C₂₀ hydrocarbyl group;
wherein each R³ is independently selected from H or a C₁-C₂₀ hydrocarbyl group;
or a pharmaceutically acceptable salt, solvate or hydrate thereof.

2. A compound as claimed in claim 1, wherein the compound has the structure: 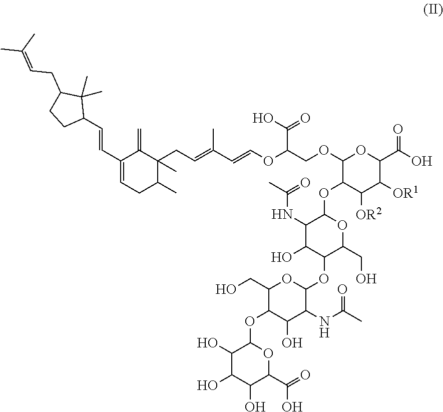
wherein R¹ is —SO₂OH, —SO₂OR or —SO₂R and R² is H, or wherein R² is —SO₂OH, —SO₂OR or —SO₂R and R¹ is H;
wherein R is a C₁-C₂₀ hydrocarbyl group;
or a pharmaceutically acceptable salt, solvate or hydrate thereof.

3. A compound as claimed in claim 1,
wherein R¹ is —SO₂OH or —SO₂OR and R² is H, or wherein R² is —SO₂OH or —SO₂OR and R¹ is H;
wherein R is a C₁-C₂₀ hydrocarbyl group;
preferably wherein R¹ is —SO₂OH and R² is H, or wherein R² is —SO₂OH and R¹ is H.

4. A compound as claimed in claim 1, wherein the compound has the structure: 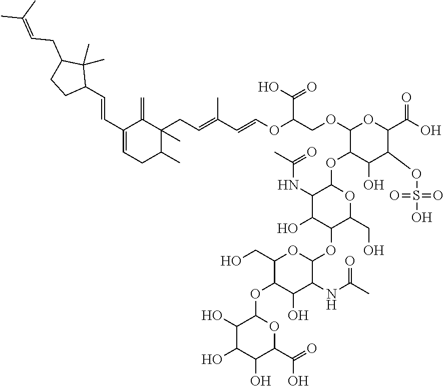 [SMILES: C=C1C(/C=C/C2CCC(CC=C(C)C)C2(C)C)=CCC(C)C1(C)C/C=C(C)/C=C/OC(COC1OC(C(=O)O)C(OS(=O)(=O)O)C(O)C1OC1OC(CO)C(OC2OC(CO)C(OC3OC(C(=O)O)C(O)C(O)C3O)C(O)C2NC(C)=O)C(O)C1NC(C)=O)C(=O)O]
or a pharmaceutically acceptable salt, solvate or hydrate thereof; or
(B) 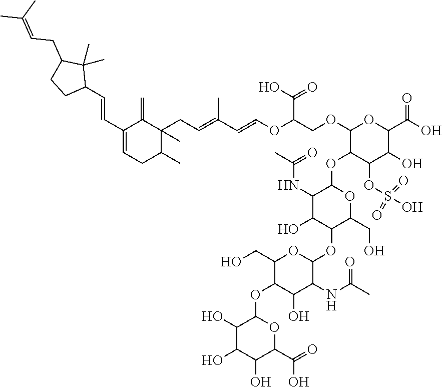 [SMILES: C=C1C(/C=C/C2CCC(CC=C(C)C)C2(C)C)=CCC(C)C1(C)C/C=C(C)/C=C/OC(COC1OC(C(=O)O)C(O)C(OS(=O)(=O)O)C1OC1OC(CO)C(OC2OC(CO)C(OC3OC(C(=O)O)C(O)C(O)C3O)C(O)C2NC(C)=O)C(O)C1NC(C)=O)C(=O)O]
or a pharmaceutically acceptable salt, solvate, or hydrate thereof.

5. (canceled)

6. A nucleic acid molecule comprising:
(a) a nucleotide sequence as shown in SEQ ID NO. 1; or
(b) a nucleotide sequence which is the complement of SEQ ID NO. 1; or
(c) a nucleotide sequence which is degenerate with SEQ ID NO. 1; or
(d) a nucleotide sequence having at least 85% sequence identity with SEQ ID NO. 1; or
(e) a part of any one of (a) to (d);
wherein said nucleic acid molecule encodes or is complementary to a nucleic acid molecule encoding one or more polypeptides, or comprises or is complementary to a nucleic acid molecule comprising one or more genetic elements, having functional activity in the synthesis of an antibiotic compound.

7. The nucleic acid molecule as claimed in claim 6, wherein:
(i) the compound is a compound of formula (I): 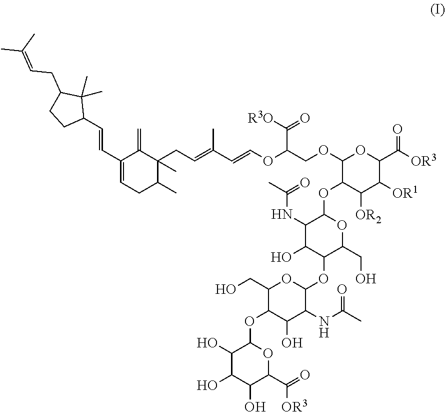
wherein R¹ is —SO₂OH, —SO₂OR or —SO₂R and R² is H, or wherein R² is —SO₂OH, —SO₂OR or —SO₂R and R¹ is H:
wherein R is a C₁-C₂₀ hydrocarbyl group;
wherein each R³ is independently selected from H or a C₁-C₂₀ hydrocarbyl group;
or a pharmaceutically acceptable salt, solvate or hydrate thereof;
(ii) encodes a biosynthetic system for the synthesis of said compound;
(iii) comprises a nucleotide sequence as shown in any one or more of SEQ ID NOs 2-29, or a nucleotide sequence which is complementary or degenerate to any one or more of SEQ ID NOs 2-29, or which has at least 85% sequence identity with any one or more of SEQ ID NOs. 2-29; and/or
(iv) comprises a nucleotide sequence which encodes an amino acid sequence as shown in any one or more of SEQ ID NOs 30-57, or an amino acid which has at least 85% sequence identity with any one or more of SEQ ID NOs. 30-57.

8-9. (canceled)

10. A polypeptide encoded by a nucleic acid molecule as defined in claim 6.

11. A recombinant construct comprising a nucleic acid molecule as defined in claim 6.

12. A vector comprising a nucleic acid molecule of claim 6 or a recombinant construct comprising the same.

13. A microbial host cell comprising:
a nucleic acid molecule as defined in claim 6, or a recombinant construct or vector comprising the same.

14. (canceled)

15. The host cell as claimed in claim 13, wherein the host cell is:
(i) a production host cell for production of an antibiotic compound and is an actinomycete;
(ii) Streptomyces sp.;
(iii) Streptomyces coelicolor;
(iv) Streptomyces coelicolor strain M145 (ATCC BAA-471);
(v) Streptomyces coelicolor strain M1152, being a derivative of (ii) comprising the modifications Δact Δred Δcpk Δcda rpoB(C₁₂₉₈T); or
(vi) Streptomyces coelicolor strain M1152ΔmatAB, being a derivative of (iii) further comprising a deletion of the locus matAB.

16. A method of producing an antibiotic compound, said method comprising introducing into a microbial host cell a nucleic acid molecule as defined in claim 6 or a recombinant construct or vector comprising said nucleic acid molecule, and allowing the nucleic acid molecule to be expressed and the compound to be synthesised by the expressed biosynthetic system.

17. The method as claimed in claim 16, wherein:
(i) the host cell is;
(a) a production host cell of an antibiotic compound and is an actinomycete;
(b) Streptomyces sp.;
(c) Streptomyces coelicolor;
(d) Streptomyces coelicolor strain M145 (ATCC BAA-471);
(e) Streptomyces coelicolor strain M1152, being a derivative of (c) comprising the modifications Δact Ared Acpk Acda rpoB (C₁₂₉₈T); or
(f) Streptomyces coelicolor strain M1152ΔmatAB, being a derivative of (d) further comprising a deletion of the locus matAB;
(ii) the method further comprises recovering the compound; and/or
(iii) the method further comprises purifying the compound.

18-19. (canceled)

20. A compound obtained or obtainable by the method as claimed in claim 16.

21. (canceled)

22. A method of treating a microbial infection in a subject or in a plant, said method comprising administering to the subject or to the plant an effective amount of a compound as claimed in claim 1.

23. The method as claimed in claim 22, wherein the microbial infection is caused by Gram-positive bacteria, preferably wherein the bacteria are Staphylococcus aureus or Enterococcus faecium, including antibiotic-resistant strains thereof.

24. (canceled)

25. A pharmaceutical composition comprising a compound as claimed in claim 1, further comprising at least one carrier, additive and/or excipient.

26. A method of controlling bacteria, comprising contacting said bacteria, or a surface or location carrying said bacteria, with a compound as claimed in claim 1.

27-28. (canceled)

29. A method of treating a microbial infection in a subject or in a plant, said method comprising administering to the subject or plant an effective amount of a compound as claimed in claim 20.

30. A pharmaceutical composition comprising a compound as claimed in claim 20, further comprising at least one carrier, additive and/or excipient.
